(19) United States
(12) Patent Application Publication
(10) Pub. No.: US 2026/0217693 A1
(43) Pub. Date: Jul. 30, 2026

(54) SRPK1 INHIBITORS AND METHODS OF USE

(71) Applicants: ALLIANTHERA (SUZHOU) BIOPHARMACEUTICAL CO., LTD., Suzhou, Jiangsu (CN); ALLIANTHERA BOSTON INC, Natick, MA (US)

(72) Inventors: Lianhai LI, Pierrefonds (CA); Cyrille F. KUHN, Ridgefield, CT (US)

(21) Appl. No.: 19/149,003

(22) PCT Filed: Jan. 19, 2024
(86) PCT No.: PCT/US2024/012097

§ 371 Date: Jul. 17, 2025

(30) Foreign Application Priority Data
Jan. 20, 2023 (WO) PCT/CN2023/073377

(51) Int. Cl. C07D 405/14; A61K 31/4545; A61K 31/496; A61K 31/497; C07D 401/14

(52) U.S. Cl. CPC C07D 405/14; A61K 31/4545; A61K 31/496; A61K 31/497; C07D 401/14

(57) ABSTRACT

Provided herein are compounds having a structure of Formula (I) and pharmaceutically acceptable salts thereof which can act as modulators of serine/arginine-rich splicing factor protein kinase-1 (SRPK1). Further disclosed herein are methods for treating cancer and vascular diseases and disorders.

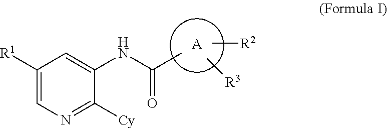

### FIELD

This disclosure relates to compounds that act as modulators of serine/arginine-rich splicing factor protein kinase-1 (SRPK1), pharmaceutical formulations thereof, and methods of using the compounds to treat cancer and vascular disorders and diseases.

### BACKGROUND

Serine/arginine-rich splicing factor protein kinase-1 (SRPK1) is a kinase that plays an important role in both constitutive and alternative splicing processes by regulating intracellular localization of splicing factors. Most nuclear messenger RNA precursors (pre-mRNA) in higher eukaryotes contain multiple introns which are precisely excised via RNA splicing. Alternative splicing can result in the production of more than one different protein from a single pre-mRNA. Recently, increasing evidence demonstrates the important role of SRPK1 in various human disorders and diseases including various cancers, vascular disease, and macular degeneration, illustrating it as a potential target for various diseases.

SRPK1 has been reported to be overexpressed in multiple cancers including prostate, breast, lung and glioma (Oncotarget. 2017, 37, 61944). In breast cancer overexpression of SRPK1 has been found to correlate with the development and progression of breast cancer and possibly resistance to taxanes (Oncotarget, 2017, 8, 103327). Several studies have further identified that inhibition/down-regulation of SRPK1 results in tumor-suppressive effects, such as reduced angiogenesis and reduced cancer cell migration. Accordingly, modulators of SRPK1 could act as potential novel anti-cancer agents.

SRPK1 is also known to play a role in regulating the expression of vascular endothelial growth factor (VEGF), a key factor in angiogenesis and vascular leakage. VEGF is up-regulated during the progression of macular degeneration. Additionally, knockdown of SRPK1 potently reduced VEGF mediated angiogenesis in vivo in tumors and inhibition of SRPK1 reduced angiogenesis in vivo. Therefore, SRPK1 is an important target in potential treatments of macular degeneration.

Thus, a need exists for compounds that modulate the SRPK1 pathway that can used in the treatment of various disorders and diseases including cancer and vascular diseases.

### SUMMARY

Provided herein are compounds having a structure of Formula (I):

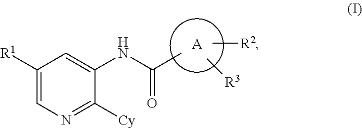

or a pharmaceutically acceptable salt thereof, wherein: ring A is a 5-10-membered heterocycle having 1, 2, or 3 ring heteroatoms independently selected from N, O, and S; Cy is a 4-10-membered heterocycle having 1, 2, or 3 ring heteroatoms independently selected from N, O, and S and optionally substituted with 1 to 4 R^{A}; each R^{A} is independently halo, OH, oxo, C₁₋₆alkyl, C₁₋₆haloalkyl, C₁₋₆hydroxyalkyl, C₀₋₆alkylene-N(R^{N})₂, C₀₋₆alkylene-C(O)N(R^{N})₂, C₀₋₆alkylene-OC(O)C₁₋₆alkyl, C₀₋₆alkylene-C(O) C₁₋₆alkyl, C₀₋₆alkylene-CO₂R^{N}, or C₀₋₃alkylene-4-8-membered heterocycle having 1, 2, 3 or 3 ring heteroatoms independently selected from N, O, and S; R¹ is H, halo, C₁₋₆alkyl, C₁₋₆haloalkyl, C₁₋₆alkoxy, C₁₋₆haloalkoxy, or C₃₋₆cycloalkyl; R² is halo, C₁₋₆alkoxy, C₁₋₆haloalkoxy, C₁₋₆alkyl, C₁₋₆alkene, C₁₋₆alkyne, C₁₋₆haloalkyl, CO₂H, or Het; Het is a 5-8-membered heterocycle having 1, 2, or 3 ring heteroatoms independently selected from N, O, and S, and is optionally substituted with 1 or 2 R^{B}; each R^{B} is independently halo, OH, oxo (═O), C₁₋₆alkyl, C₁₋₆haloalkyl, C₁₋₆alkoxy, C₁₋₆haloalkoxy, C₀₋₆alkylene-N(R^{N})₂, C₀₋₆alkylene-4-8-membered heterocycle having 1, 2, 3 or 3 ring heteroatoms independently selected from N, O, and S; R³ is H, halo, or C₁₋₆alkyl; and each R^{N} is independently H or C₁₋₃alkyl.

Further provided herein are pharmaceutical compositions comprising the compounds as disclosed herein. Also provided are methods of treating or preventing a disease or disorder associated with aberrant serine/arginine-rich splicing factor protein kinase-1 (SRPK1) activity in a subject, comprising administering to the subject a therapeutically effective amount of a compound as disclosed herein.

Further aspects and advantages will be apparent to those of ordinary skill in the art from a review of the following detailed description, taken in conjunction with the drawings. While the compounds and methods disclosed herein are susceptible of cases in various forms, the description hereafter includes specific cases with the understanding that the disclosure is illustrative and is not intended to limit the invention to the specific cases described herein.

### DETAILED DESCRIPTION

There is a need for novel SRPK1 inhibitors to provide new and effective therapies for diseases and disorders, such as cancer and vascular diseases and disorders. Provided herein are compounds that can act as SRPK1 modulators, such as compounds of Formula (I):

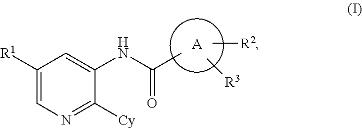

and pharmaceutically acceptable salts thereof, wherein ring A, Cy, R¹, R², and R³ are as described herein.

### Compounds of the Disclosure

Disclosed herein are compounds having a structure of Formula (I):

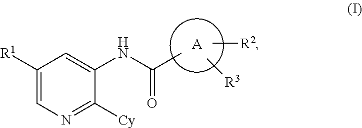

and pharmaceutically acceptable salts thereof wherein:
ring A is a 5-10-membered heterocycle having 1, 2, or 3 ring heteroatoms independently selected from N, O, and S;
Cy is a 4-10-membered heterocycle having 1, 2, or 3 ring heteroatoms independently selected from N, O, and S and optionally substituted with 1 to 4 R^{A};
each R^{A} is independently halo, OH, oxo, C₁₋₆alkyl, C₁₋₆haloalkyl, C₁₋₆hydroxyalkyl, C₀₋₆alkylene-N(R^{N})₂, C₀₋₆alkylene-C(O)N(R^{N})₂, C₀₋₆alkylene-OC(O)C₁₋₆alkyl, C₀₋₆alkylene-C(O) C₁₋₆alkyl, C₀₋₆alkylene-CO₂R^{N}, or C₀₋₆alkylene-4-8-membered heterocycle having 1, 2, 3 or 3 ring heteroatoms independently selected from N, O, and S;
R¹ is H, halo, C₁₋₆alkyl, C₁₋₆haloalkyl, C₁₋₆alkoxy, C₁₋₆haloalkoxy, or C₃₋₆cycloalkyl;
R² is halo, C₁₋₆alkoxy, C₁₋₆haloalkoxy, C₁₋₆alkyl, C₂₋₆alkene, C₂₋₆alkyne, C₁₋₆haloalkyl, CO₂H, or Het;
Het is a 5-8-membered heterocycle having 1, 2, or 3 ring heteroatoms independently selected from N, O, and S, and is optionally substituted with 1 or 2 R^{B};
each R^{B} is independently halo, OH, oxo (═O), C₁₋₆alkyl, C₁₋₆haloalkyl, C₁₋₆alkoxy, C₁₋₆haloalkoxy, C₀₋₆alkylene-N(R^{N})₂, or C₀₋₆alkylene-4-8-membered heterocycle having 1, 2, 3 or 3 ring heteroatoms independently selected from N, O, and S;
R³ is H, halo, or C₁₋₆alkyl; and
each R^{N} is independently H or C₁₋₃alkyl.

In compounds of Formula (I), ring A can be a 5-10-membered heterocycle having 1, 2, or 3 ring heteroatoms independently selected from N, O, and S. In various cases, ring A is aromatic, and is optionally a 5- or 6-membered aromatic ring. In various cases, ring A is furan, oxazole, isoxazole, thiophene, thiazole, imidazole, pyrazole, triazole, pyridine, pyrimidine, pyridazine, or pyrazine. In various cases, ring A is furan. In some cases, ring A is 2-furan. In some cases, ring A is pyridine. In some cases, ring A is pyrazole.

In various cases, the compound has a structure of Formula (II):

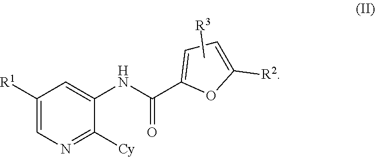

In various cases, the compound has a structure of Formula (III):

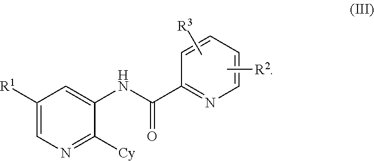

As disclosed herein, Cy can be a 4-10-membered heterocycle having 1, 2, or 3 ring heteroatoms independently selected from N, O, and S. In various cases, Cy can be optionally substituted with 1 to 4 R^{A}. In many cases, Cy is unsubstituted (i.e., no R^{A} groups are present). In some cases, Cy is substituted with 1 R^{A}. In some cases, Cy is substituted with 2 R^{A}. In various cases, Cy is azetidine, pyrrolidine, piperidine, piperazine, azepane, morpholine, thiomorpholine, pyrazole, pyridine, pyrazine, pyrimidine, pyridazine, 2,4,6,7-tetrahydro-pyrazolo[4,3-c]pyridine, 2-oxa-7-azaspiro[3.5]nonane, 7-azaspiro[3.5]nonane, 2-oxa-8-azaspiro[4.5]decane, 2,8-diazaspiro[4.5]decane, or 1,8-diazaspiro[4.5]decane. In some cases,

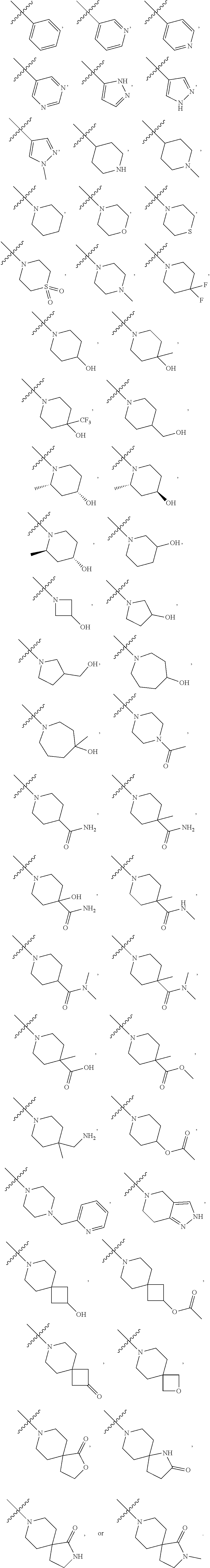

As disclosed herein, each R^{A} can independently be halo, OH, oxo, C₁₋₆alkyl, C₁₋₆haloalkyl, C₁₋₆hydroxyalkyl, C₀₋₆alkylene-N(R^{N})₂, C₀₋₆alkylene-C(O)N(R^{N})₂, C₀₋₆alkylene-OC(O)C₁₋₆alkyl, C₀₋₆alkylene-C(O) C₁₋₆alkyl, C₀₋₆alkylene-CO₂R^{N}, or C₀₋₃alkylene-4-8-membered heterocycle having 1, 2, 3 or 3 ring heteroatoms independently selected from N, O, and S. In various cases, at least one R^{A} is OH, F, CH₃, C(O)N(R^{N})₂, CH₂OH, oxo, CF₃, OC(O)CH₃, CO₂CH₃, CO₂H, CH₂-pyridine, C(O)CH₃, or CH₂N(R^{N})₂.

As disclosed herein, R¹ can be H, halo, C₁₋₆alkyl, C₁₋₆haloalkyl, C₁₋₆alkoxy, C₁₋₆haloalkoxy, or C₃₋₆cycloalkyl. In various cases, R¹ is halo. In some cases, R¹ is CF₃. In some cases, R¹ is cyclopropyl.

As disclosed herein, R² can be halo, C₁₋₆alkoxy, C₁₋₆haloalkoxy, C₁₋₆alkyl, C₂₋₆alkene, C₂₋₆alkyne, C₁₋₆haloalkyl, CO₂H, or Het. In some cases, R² is halo, C₁₋₆alkoxy, C₁₋₆haloalkoxy, C₁₋₆alkyl, C₁₋₆haloalkyl, CO₂H, or Het. In various cases, R² is halo, C₁₋₃alkoxy, or C₁₋₃haloalkyl. In some cases, R² is Het.

As disclosed herein, Het can be a 5-8-membered heterocycle having 1, 2, or 3 ring heteroatoms independently selected from N, O, and S. In various cases, Het can be optionally substituted with 1 to 4 R^{B}. In some cases, Het is unsubstituted (no R^{B} groups are present). In some cases, Het is substituted with 1 R^{B}. In some cases, Het is substituted with 2 R^{B}. In various cases, Het is pyridine, pyrazole, tetrahydropyran, pyrazine, pyrimidine, pyridazine, or piperidine. In some cases, Het is

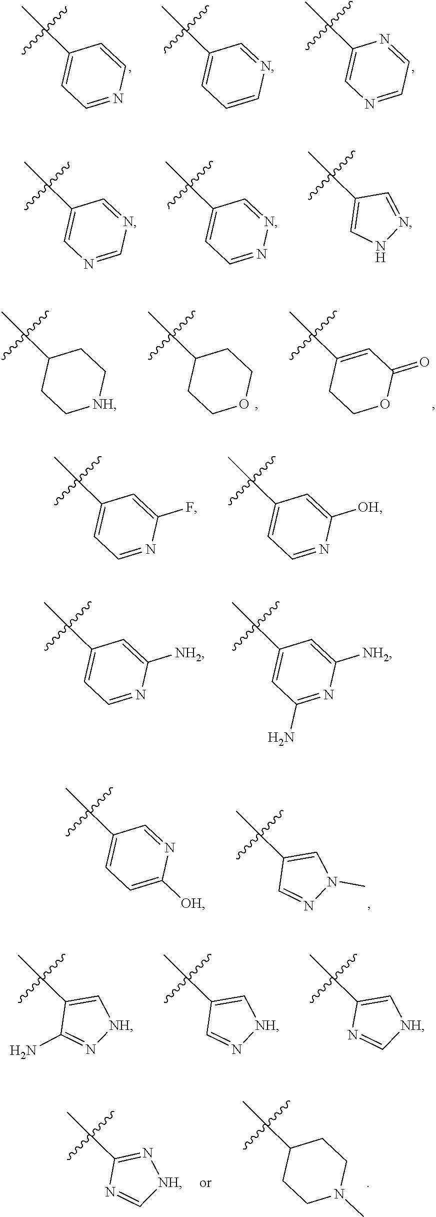

As disclosed herein, each R^{B} can independently be halo, OH, oxo (═O), C₁₋₆alkyl, C₁₋₆haloalkyl, C₁₋₆alkoxy, C₁₋₆haloalkoxy, C₀₋₆alkylene-N(R^{N})₂, or C₀₋₆alkylene-4-8-membered heterocycle having 1, 2, 3 or 3 ring heteroatoms independently selected from N, O, and S. In various cases, at least one R^{B} is NH₂, OH, CH₃, or F.

As disclosed herein, R³ can be H, halo, or C₁₋₆alkyl. In various cases, R³ is H.

As disclosed herein, each R^{N} can independently be H or C₁₋₃alkyl. In various cases, each R^{N} is H or methyl.

Compounds as disclosed herein include those as provided in Table A, or a pharmaceutically acceptable salt thereof. For the avoidance of doubt, all stereocenters shown in the compounds of Table A are relative, not absolute stereochemistry.

TABLE A

| | Structure |
|---|---|
| 1 | 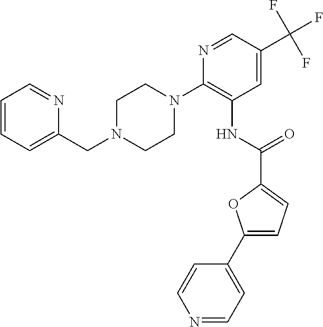 [SMILES: O=C(Nc1cc(C(F)(F)F)cnc1N1CCN(Cc2ccccn2)CC1)c1ccc(-c2ccncc2)o1] |
| 2 | 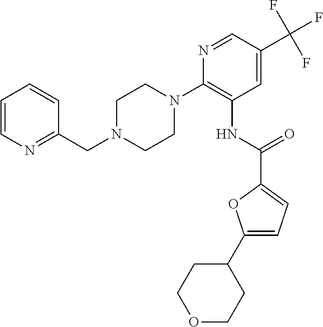 [SMILES: O=C(Nc1cc(C(F)(F)F)cnc1N1CCN(Cc2ccccn2)CC1)c1ccc(C2CCOCC2)o1] |
| 3 | 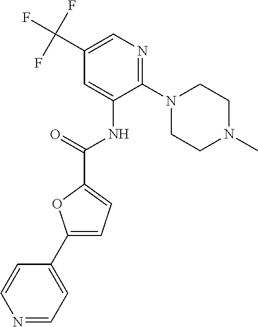 [SMILES: CN1CCN(c2ncc(C(F)(F)F)cc2NC(=O)c2ccc(-c3ccncc3)o2)CC1] |
| 4 | 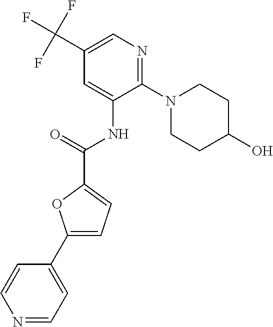 [SMILES: O=C(Nc1cc(C(F)(F)F)cnc1N1CCC(O)CC1)c1ccc(-c2ccncc2)o1] |
| 5 | 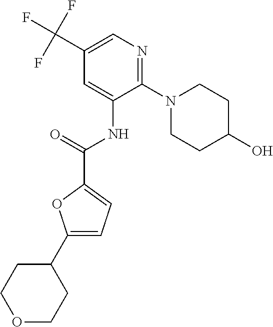 [SMILES: O=C(Nc1cc(C(F)(F)F)cnc1N1CCC(O)CC1)c1ccc(C2CCOCC2)o1] |
| 6 | 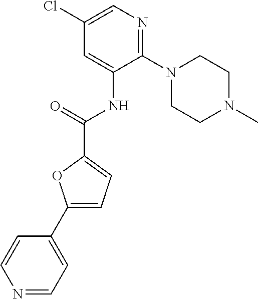 [SMILES: CN1CCN(c2ncc(Cl)cc2NC(=O)c2ccc(-c3ccncc3)o2)CC1] |
| 7 | 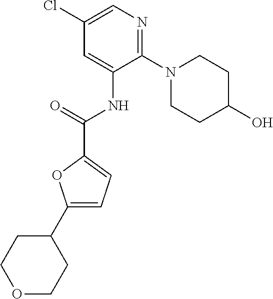 [SMILES: O=C(Nc1cc(Cl)cnc1N1CCC(O)CC1)c1ccc(C2CCOCC2)o1] |
| 8 | 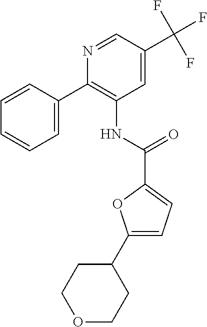 [SMILES: O=C(Nc1cc(C(F)(F)F)cnc1-c1ccccc1)c1ccc(C2CCOCC2)o1] |
| 9 | 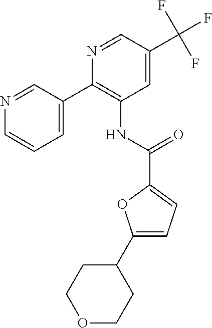 [SMILES: O=C(Nc1cc(C(F)(F)F)cnc1-c1cccnc1)c1ccc(C2CCOCC2)o1] |
| 10 | 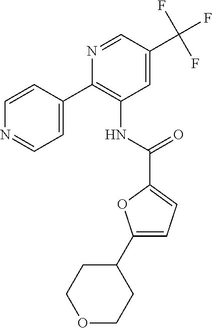 [SMILES: O=C(Nc1cc(C(F)(F)F)cnc1-c1ccncc1)c1ccc(C2CCOCC2)o1] |
| 11 | 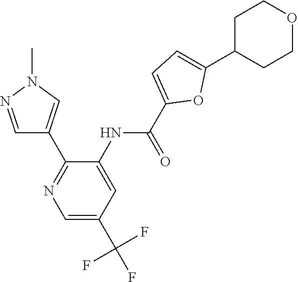 [SMILES: Cn1cc(-c2ncc(C(F)(F)F)cc2NC(=O)c2ccc(C3CCOCC3)o2)cn1] |
| 12 | 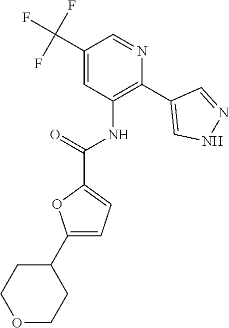 [SMILES: O=C(Nc1cc(C(F)(F)F)cnc1-c1cn[nH]c1)c1ccc(C2CCOCC2)o1] |
| 13 | 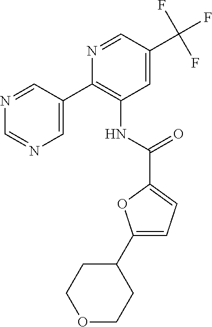 [SMILES: O=C(Nc1cc(C(F)(F)F)cnc1-c1cncnc1)c1ccc(C2CCOCC2)o1] |
| 14 | 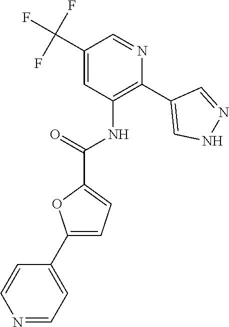 [SMILES: O=C(Nc1cc(C(F)(F)F)cnc1-c1cn[nH]c1)c1ccc(-c2ccncc2)o1] |
| 15 | 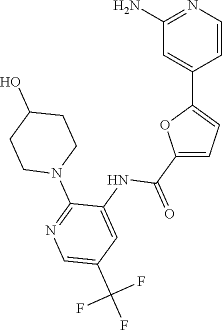 [SMILES: Nc1cc(-c2ccc(C(=O)Nc3cc(C(F)(F)F)cnc3N3CCC(O)CC3)o2)ccn1] |
| 16 | 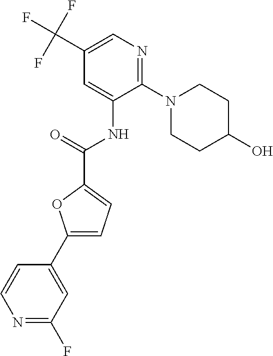 [SMILES: O=C(Nc1cc(C(F)(F)F)cnc1N1CCC(O)CC1)c1ccc(-c2ccnc(F)c2)o1] |
| 17 | 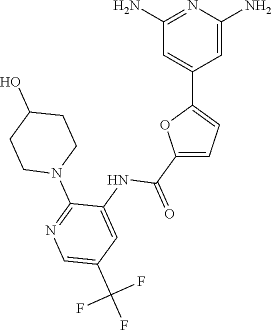 [SMILES: Nc1cc(-c2ccc(C(=O)Nc3cc(C(F)(F)F)cnc3N3CCC(O)CC3)o2)cc(N)n1] |
| 18 | 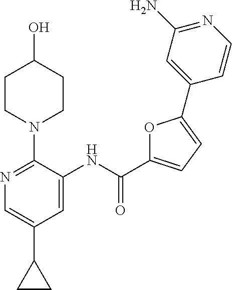 [SMILES: Nc1cc(-c2ccc(C(=O)Nc3cc(C4CC4)cnc3N3CCC(O)CC3)o2)ccn1] |
| 19 | 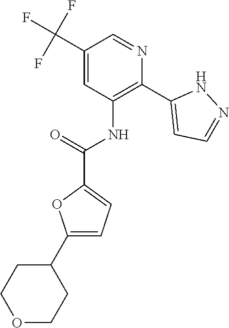 [SMILES: O=C(Nc1cc(C(F)(F)F)cnc1-c1ccn[nH]1)c1ccc(C2CCOCC2)o1] |
| 20 | 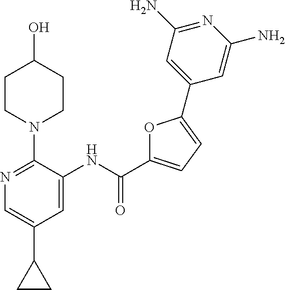 [SMILES: Nc1cc(-c2ccc(C(=O)Nc3cc(C4CC4)cnc3N3CCC(O)CC3)o2)cc(N)n1] |
| 21 | 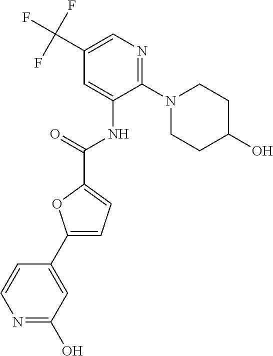 [SMILES: O=C(Nc1cc(C(F)(F)F)cnc1N1CCC(O)CC1)c1ccc(-c2ccnc(O)c2)o1] |
| 22 | 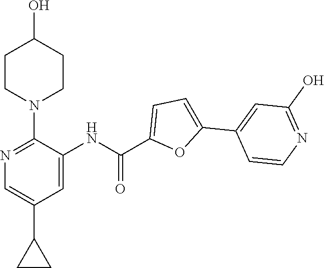 [SMILES: O=C(Nc1cc(C2CC2)cnc1N1CCC(O)CC1)c1ccc(-c2ccnc(O)c2)o1] |
| 23 | 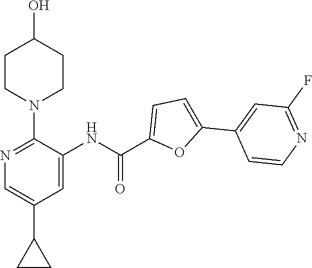 [SMILES: O=C(Nc1cc(C2CC2)cnc1N1CCC(O)CC1)c1ccc(-c2ccnc(F)c2)o1] |
| 24 | 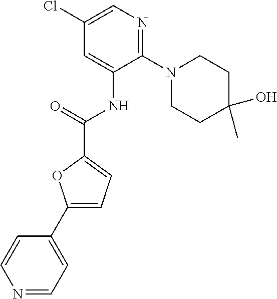 [SMILES: CC1(O)CCN(c2ncc(Cl)cc2NC(=O)c2ccc(-c3ccncc3)o2)CC1] |
| 25 | 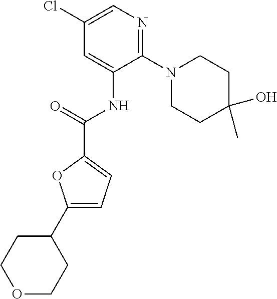 [SMILES: CC1(O)CCN(c2ncc(Cl)cc2NC(=O)c2ccc(C3CCOCC3)o2)CC1] |
| 26 | 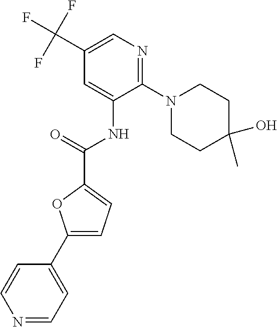 [SMILES: CC1(O)CCN(c2ncc(C(F)(F)F)cc2NC(=O)c2ccc(-c3ccncc3)o2)CC1] |
| 27 | 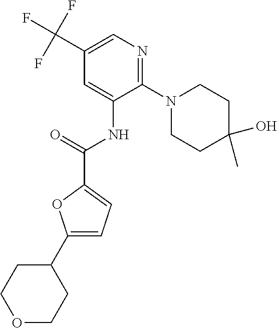 [SMILES: CC1(O)CCN(c2ncc(C(F)(F)F)cc2NC(=O)c2ccc(C3CCOCC3)o2)CC1] |
| 28 | 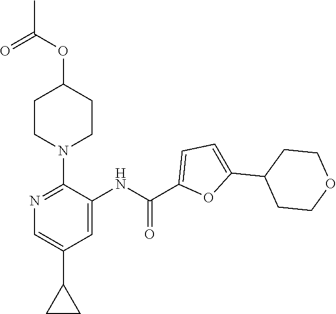 [SMILES: CC(=O)OC1CCN(c2ncc(C3CC3)cc2NC(=O)c2ccc(C3CCOCC3)o2)CC1] |
| 29 | 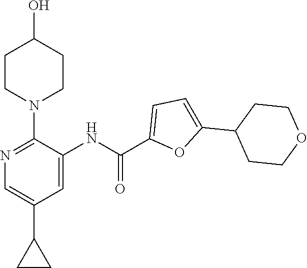 [SMILES: O=C(Nc1cc(C2CC2)cnc1N1CCC(O)CC1)c1ccc(C2CCOCC2)o1] |
| 30 | 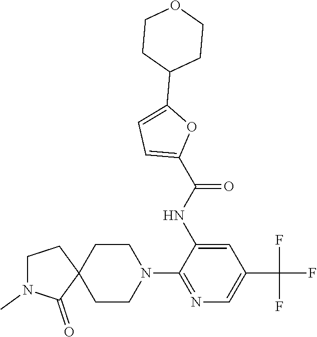 [SMILES: CN1CCC2(CCN(c3ncc(C(F)(F)F)cc3NC(=O)c3ccc(C4CCOCC4)o3)CC2)C1=O] |
| 31 | 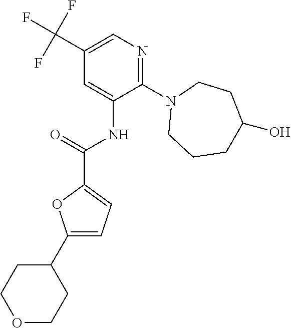 [SMILES: O=C(Nc1cc(C(F)(F)F)cnc1N1CCCC(O)CC1)c1ccc(C2CCOCC2)o1] |
| 32 | 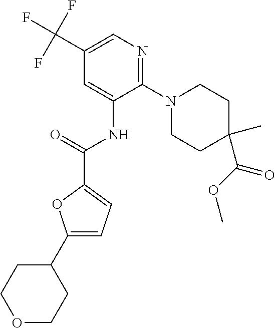 [SMILES: COC(=O)C1(C)CCN(c2ncc(C(F)(F)F)cc2NC(=O)c2ccc(C3CCOCC3)o2)CC1] |
| 33 | 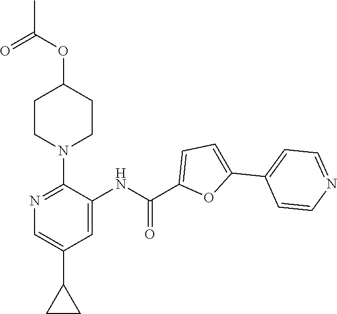 [SMILES: CC(=O)OC1CCN(c2ncc(C3CC3)cc2NC(=O)c2ccc(-c3ccncc3)o2)CC1] |
| 34 | 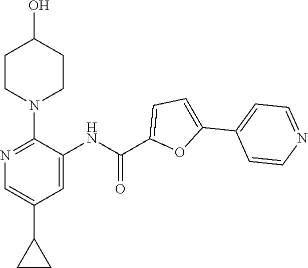 [SMILES: O=C(Nc1cc(C2CC2)cnc1N1CCC(O)CC1)c1ccc(-c2ccncc2)o1] |
| 35 | 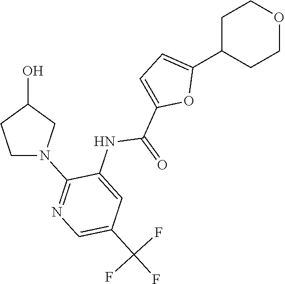 [SMILES: O=C(Nc1cc(C(F)(F)F)cnc1N1CCC(O)C1)c1ccc(C2CCOCC2)o1] |
| 36 | 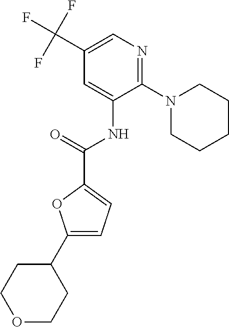 [SMILES: O=C(Nc1cc(C(F)(F)F)cnc1N1CCCCC1)c1ccc(C2CCOCC2)o1] |
| 37 | 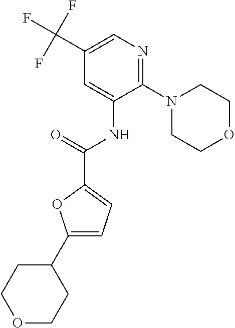 [SMILES: O=C(Nc1cc(C(F)(F)F)cnc1N1CCOCC1)c1ccc(C2CCOCC2)o1] |
| 38 | 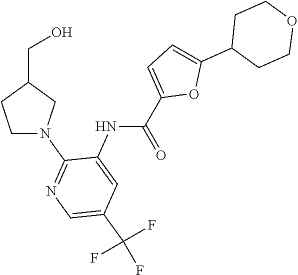 [SMILES: O=C(Nc1cc(C(F)(F)F)cnc1N1CCC(CO)C1)c1ccc(C2CCOCC2)o1] |
| 39 | 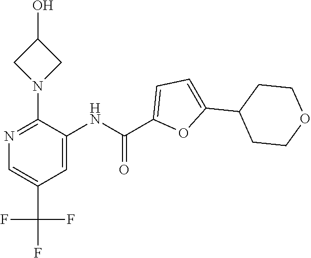 [SMILES: O=C(Nc1cc(C(F)(F)F)cnc1N1CC(O)C1)c1ccc(C2CCOCC2)o1] |
| 40 | 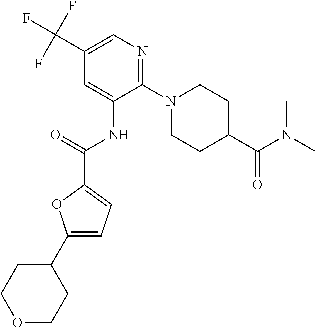 [SMILES: CN(C)C(=O)C1CCN(c2ncc(C(F)(F)F)cc2NC(=O)c2ccc(C3CCOCC3)o2)CC1] |
| 41 | 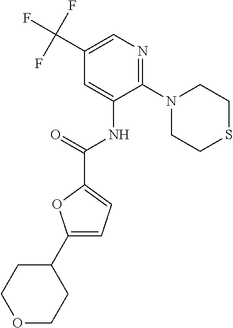 [SMILES: O=C(Nc1cc(C(F)(F)F)cnc1N1CCSCC1)c1ccc(C2CCOCC2)o1] |
| 42 | 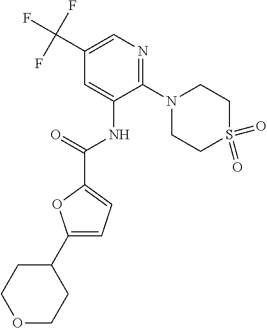 [SMILES: O=C(Nc1cc(C(F)(F)F)cnc1N1CCS(=O)(=O)CC1)c1ccc(C2CCOCC2)o1] |
| 43 | 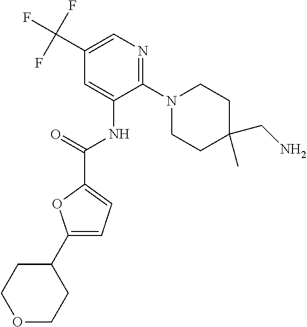 [SMILES: CC1(CN)CCN(c2ncc(C(F)(F)F)cc2NC(=O)c2ccc(C3CCOCC3)o2)CC1] |
| 44 | 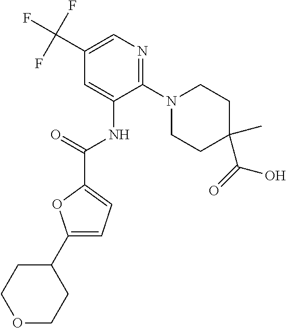 [SMILES: CC1(C(=O)O)CCN(c2ncc(C(F)(F)F)cc2NC(=O)c2ccc(C3CCOCC3)o2)CC1] |
| 45 | 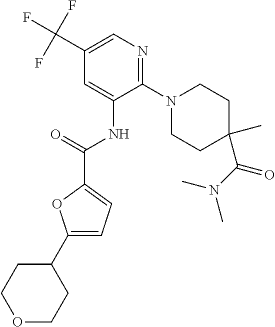 [SMILES: CN(C)C(=O)C1(C)CCN(c2ncc(C(F)(F)F)cc2NC(=O)c2ccc(C3CCOCC3)o2)CC1] |
| 46 | 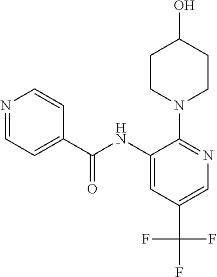 [SMILES: O=C(Nc1cc(C(F)(F)F)cnc1N1CCC(O)CC1)c1ccncc1] |
| 47 | 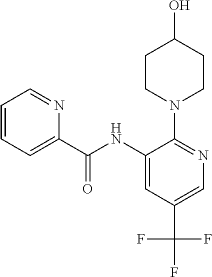 [SMILES: O=C(Nc1cc(C(F)(F)F)cnc1N1CCC(O)CC1)c1ccccn1] |
| 48 | 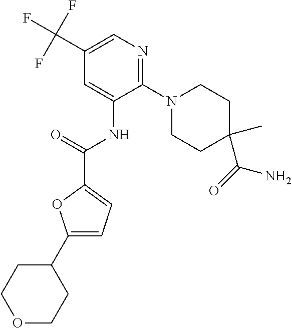 [SMILES: CC1(C(N)=O)CCN(c2ncc(C(F)(F)F)cc2NC(=O)c2ccc(C3CCOCC3)o2)CC1] |
| 49 | 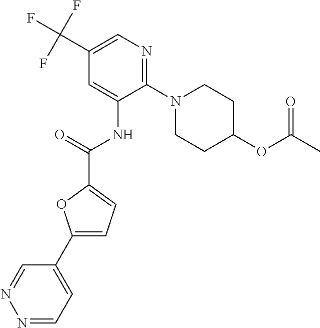 [SMILES: CC(=O)OC1CCN(c2ncc(C(F)(F)F)cc2NC(=O)c2ccc(-c3ccnnc3)o2)CC1] |
| 50 | 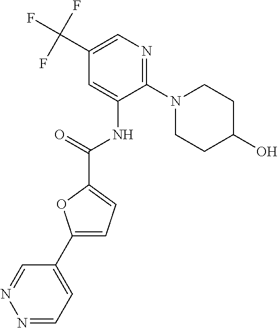 [SMILES: O=C(Nc1cc(C(F)(F)F)cnc1N1CCC(O)CC1)c1ccc(-c2ccnnc2)o1] |
| 51 | 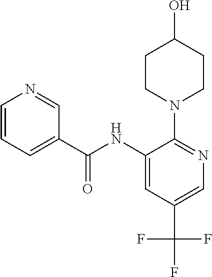 [SMILES: O=C(Nc1cc(C(F)(F)F)cnc1N1CCC(O)CC1)c1cccnc1] |
| 52 | 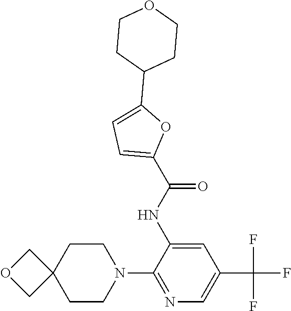 [SMILES: O=C(Nc1cc(C(F)(F)F)cnc1N1CCC2(CC1)COC2)c1ccc(C2CCOCC2)o1] |
| 53 | 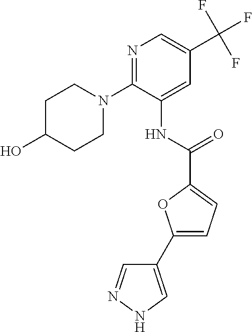 [SMILES: O=C(Nc1cc(C(F)(F)F)cnc1N1CCC(O)CC1)c1ccc(-c2cn[nH]c2)o1] |
| 54 | 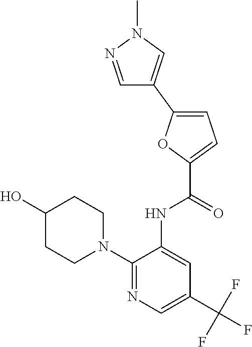 [SMILES: Cn1cc(-c2ccc(C(=O)Nc3cc(C(F)(F)F)cnc3N3CCC(O)CC3)o2)cn1] |
| 55 | 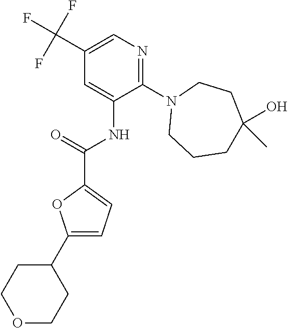 [SMILES: CC1(O)CCCN(c2ncc(C(F)(F)F)cc2NC(=O)c2ccc(C3CCOCC3)o2)CC1] |
| 56 | 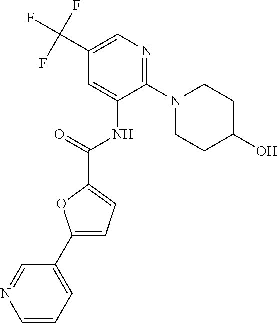 [SMILES: O=C(Nc1cc(C(F)(F)F)cnc1N1CCC(O)CC1)c1ccc(-c2cccnc2)o1] |
| 57 | 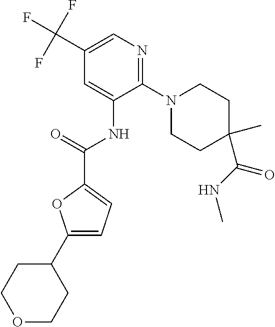 [SMILES: CNC(=O)C1(C)CCN(c2ncc(C(F)(F)F)cc2NC(=O)c2ccc(C3CCOCC3)o2)CC1] |
| 58 | 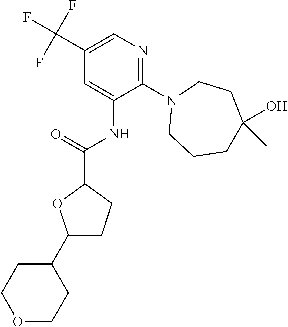 [SMILES: CC1(O)CCCN(c2ncc(C(F)(F)F)cc2NC(=O)C2CCC(C3CCOCC3)O2)CC1] |
| 59 | 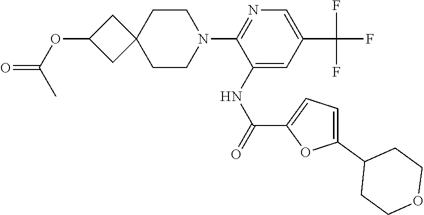 [SMILES: CC(=O)OC1CC2(CCN(c3ncc(C(F)(F)F)cc3NC(=O)c3ccc(C4CCOCC4)o3)CC2)C1] |
| 60 | 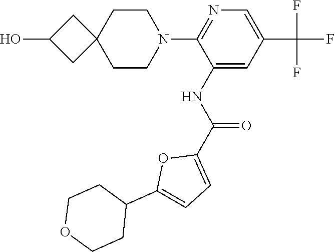 [SMILES: O=C(Nc1cc(C(F)(F)F)cnc1N1CCC2(CC1)CC(O)C2)c1ccc(C2CCOCC2)o1] |
| 61 | 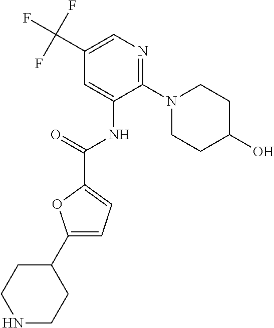 [SMILES: O=C(Nc1cc(C(F)(F)F)cnc1N1CCC(O)CC1)c1ccc(C2CCNCC2)o1] |
| 62 | 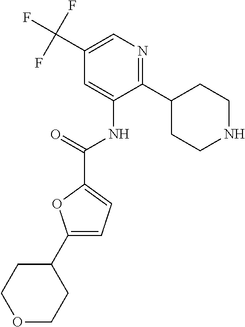 [SMILES: O=C(Nc1cc(C(F)(F)F)cnc1C1CCNCC1)c1ccc(C2CCOCC2)o1] |
| 63 | 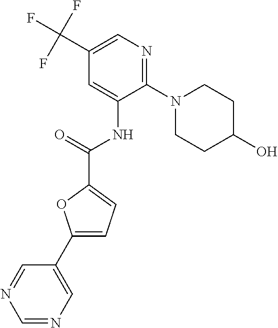 [SMILES: O=C(Nc1cc(C(F)(F)F)cnc1N1CCC(O)CC1)c1ccc(-c2cncnc2)o1] |
| 64 | 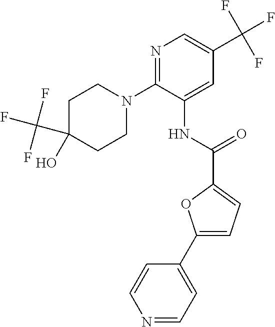 [SMILES: O=C(Nc1cc(C(F)(F)F)cnc1N1CCC(O)(C(F)(F)F)CC1)c1ccc(-c2ccncc2)o1] |
| 65 | 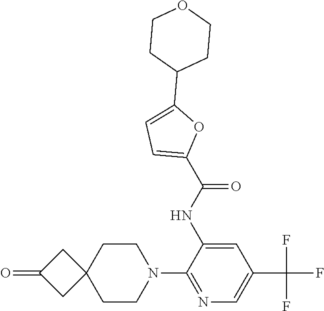 [SMILES: O=C1CC2(CCN(c3ncc(C(F)(F)F)cc3NC(=O)c3ccc(C4CCOCC4)o3)CC2)C1] |
| 66 | 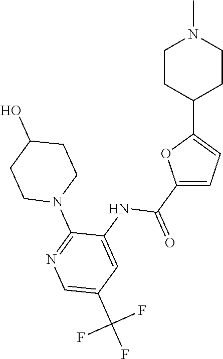 [SMILES: CN1CCC(c2ccc(C(=O)Nc3cc(C(F)(F)F)cnc3N3CCC(O)CC3)o2)CC1] |
| 67 | 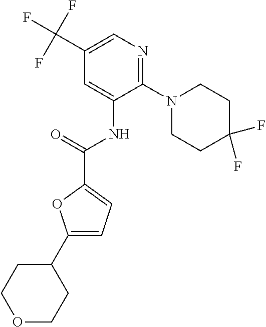 [SMILES: O=C(Nc1cc(C(F)(F)F)cnc1N1CCC(F)(F)CC1)c1ccc(C2CCOCC2)o1] |
| 68 | 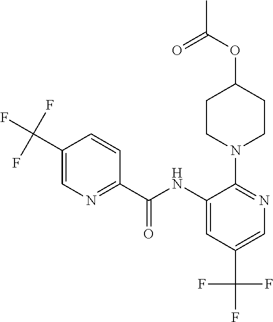 [SMILES: CC(=O)OC1CCN(c2ncc(C(F)(F)F)cc2NC(=O)c2ccc(C(F)(F)F)cn2)CC1] |
| 69 | 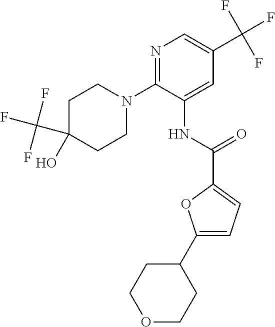 [SMILES: O=C(Nc1cc(C(F)(F)F)cnc1N1CCC(O)(C(F)(F)F)CC1)c1ccc(C2CCOCC2)o1] |
| 70 | 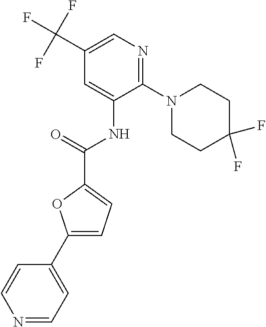 [SMILES: O=C(Nc1cc(C(F)(F)F)cnc1N1CCC(F)(F)CC1)c1ccc(-c2ccncc2)o1] |
| 71 | 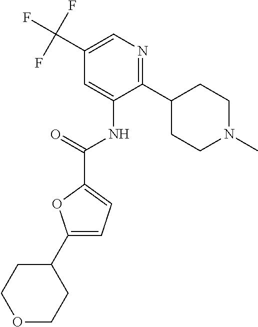 [SMILES: CN1CCC(c2ncc(C(F)(F)F)cc2NC(=O)c2ccc(C3CCOCC3)o2)CC1] |
| 72 | 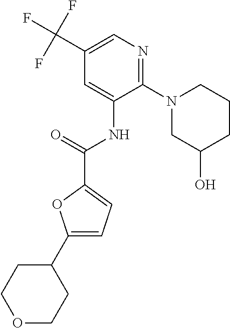 [SMILES: O=C(Nc1cc(C(F)(F)F)cnc1N1CCCC(O)C1)c1ccc(C2CCOCC2)o1] |
| 73 | 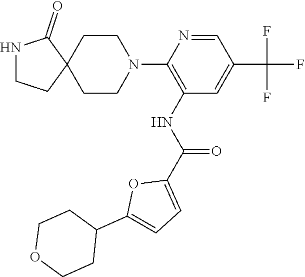 [SMILES: O=C(Nc1cc(C(F)(F)F)cnc1N1CCC2(CCNC2=O)CC1)c1ccc(C2CCOCC2)o1] |
| 74 | 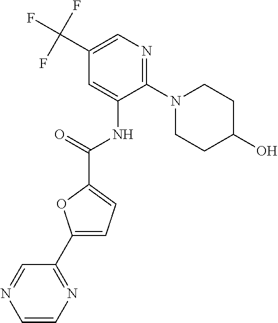 [SMILES: O=C(Nc1cc(C(F)(F)F)cnc1N1CCC(O)CC1)c1ccc(-c2cnccn2)o1] |
| 75 | 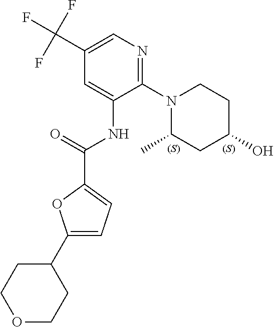 |
| 76 | 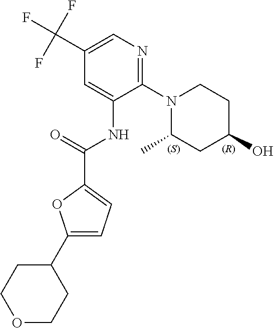 |
| 77 | 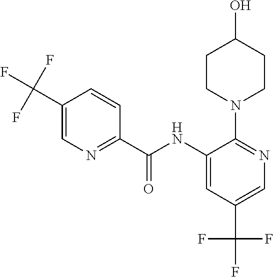 [SMILES: O=C(Nc1cc(C(F)(F)F)cnc1N1CCC(O)CC1)c1ccc(C(F)(F)F)cn1] |
| 78 | 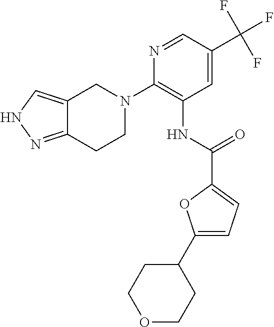 [SMILES: O=C(Nc1cc(C(F)(F)F)cnc1N1CCc2n[nH]cc2C1)c1ccc(C2CCOCC2)o1] |
| 79 | 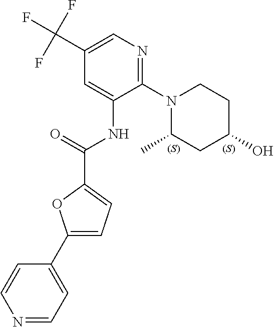 |
| 80 | 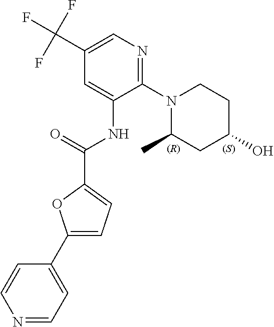 |
| 81 | 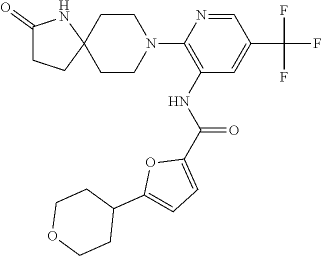 [SMILES: O=C1CCC2(CCN(c3ncc(C(F)(F)F)cc3NC(=O)c3ccc(C4CCOCC4)o3)CC2)N1] |
| 82 | 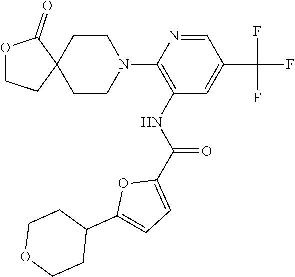 [SMILES: O=C(Nc1cc(C(F)(F)F)cnc1N1CCC2(CCOC2=O)CC1)c1ccc(C2CCOCC2)o1] |
| 83 | 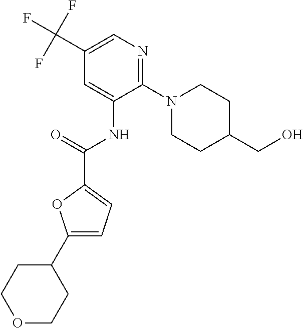 [SMILES: O=C(Nc1cc(C(F)(F)F)cnc1N1CCC(CO)CC1)c1ccc(C2CCOCC2)o1] |
| 84 | 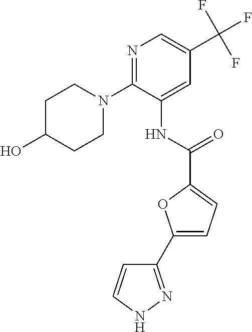 [SMILES: O=C(Nc1cc(C(F)(F)F)cnc1N1CCC(O)CC1)c1ccc(-c2cc[nH]n2)o1] |
| 85 | 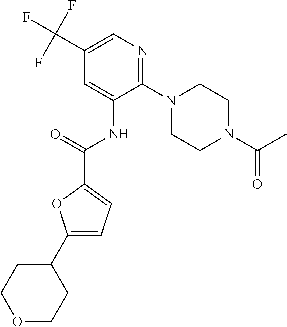 [SMILES: CC(=O)N1CCN(c2ncc(C(F)(F)F)cc2NC(=O)c2ccc(C3CCOCC3)o2)CC1] |
| 86 | 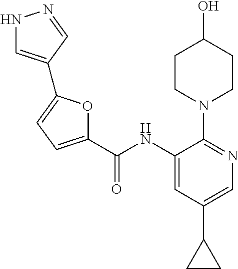 [SMILES: O=C(Nc1cc(C2CC2)cnc1N1CCC(O)CC1)c1ccc(-c2cn[nH]c2)o1] |
| 87 | 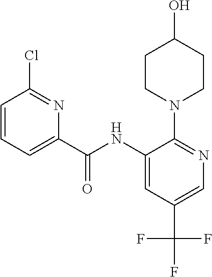 [SMILES: O=C(Nc1cc(C(F)(F)F)cnc1N1CCC(O)CC1)c1cccc(Cl)n1] |
| 88 | 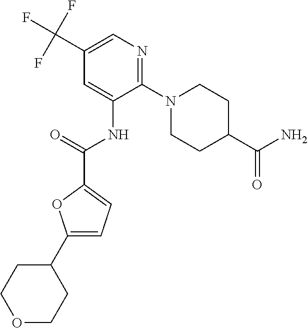 [SMILES: NC(=O)C1CCN(c2ncc(C(F)(F)F)cc2NC(=O)c2ccc(C3CCOCC3)o2)CC1] |
| 89 | 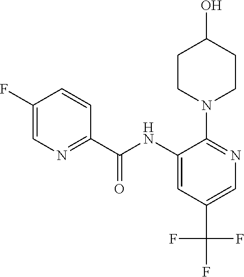 [SMILES: O=C(Nc1cc(C(F)(F)F)cnc1N1CCC(O)CC1)c1ccc(F)cn1] |
| 90 | 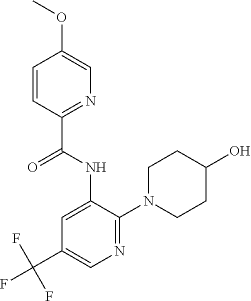 [SMILES: COc1ccc(C(=O)Nc2cc(C(F)(F)F)cnc2N2CCC(O)CC2)nc1] |
| 91 | 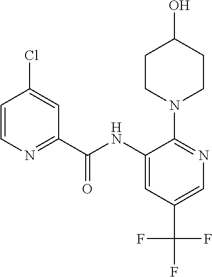 [SMILES: O=C(Nc1cc(C(F)(F)F)cnc1N1CCC(O)CC1)c1cc(Cl)ccn1] |
| 92 | 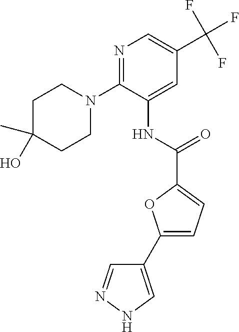 [SMILES: CC1(O)CCN(c2ncc(C(F)(F)F)cc2NC(=O)c2ccc(-c3cn[nH]c3)o2)CC1] |
| 93 | 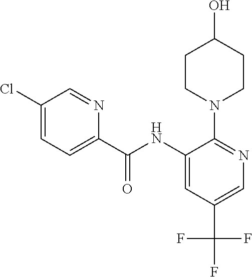 [SMILES: O=C(Nc1cc(C(F)(F)F)cnc1N1CCC(O)CC1)c1ccc(Cl)cn1] |
| 94 | 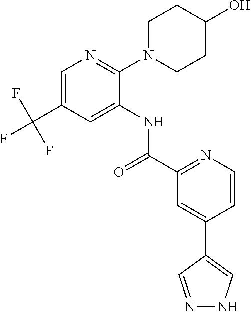 [SMILES: O=C(Nc1cc(C(F)(F)F)cnc1N1CCC(O)CC1)c1cc(-c2cn[nH]c2)ccn1] |
| 95 | 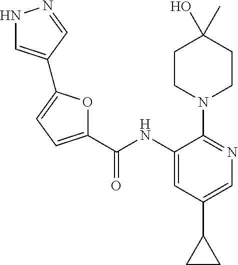 [SMILES: CC1(O)CCN(c2ncc(C3CC3)cc2NC(=O)c2ccc(-c3cn[nH]c3)o2)CC1] |
| 96 | 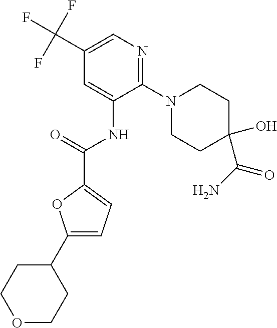 [SMILES: NC(=O)C1(O)CCN(c2ncc(C(F)(F)F)cc2NC(=O)c2ccc(C3CCOCC3)o2)CC1] |
| 97 | 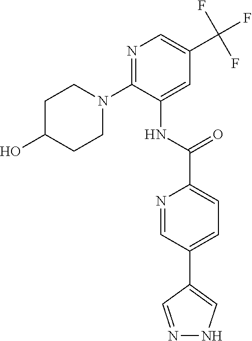 [SMILES: O=C(Nc1cc(C(F)(F)F)cnc1N1CCC(O)CC1)c1ccc(-c2cn[nH]c2)cn1] |
| 98 | 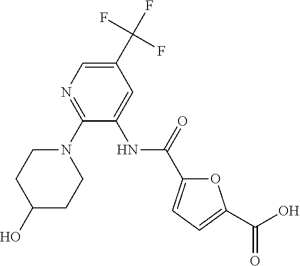 [SMILES: O=C(O)c1ccc(C(=O)Nc2cc(C(F)(F)F)cnc2N2CCC(O)CC2)o1] |
| 99 | 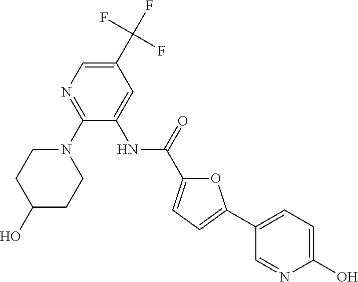 [SMILES: O=C(Nc1cc(C(F)(F)F)cnc1N1CCC(O)CC1)c1ccc(-c2ccc(O)nc2)o1] |
| 100 | 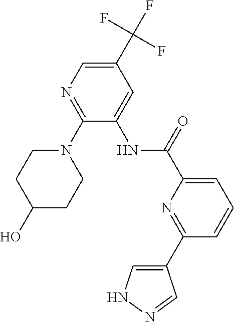 [SMILES: O=C(Nc1cc(C(F)(F)F)cnc1N1CCC(O)CC1)c1cccc(-c2cn[nH]c2)n1] |
| 101 | 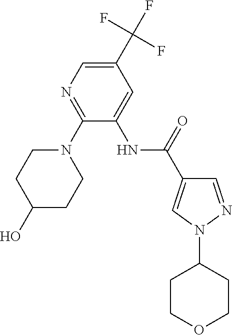 [SMILES: O=C(Nc1cc(C(F)(F)F)cnc1N1CCC(O)CC1)c1cnn(C2CCOCC2)c1] |
| 102 | 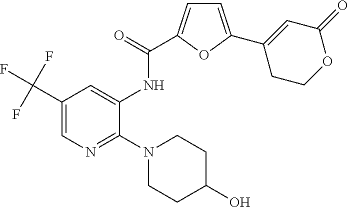 [SMILES: O=C1C=C(c2ccc(C(=O)Nc3cc(C(F)(F)F)cnc3N3CCC(O)CC3)o2)CCO1] |
| 103 | 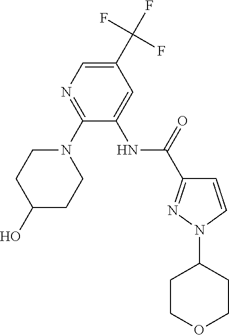 [SMILES: O=C(Nc1cc(C(F)(F)F)cnc1N1CCC(O)CC1)c1ccn(C2CCOCC2)n1] |
| 104 | 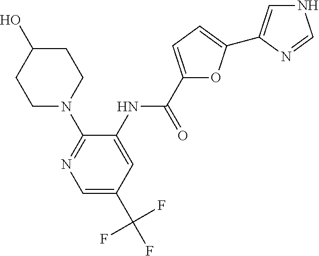 [SMILES: O=C(Nc1cc(C(F)(F)F)cnc1N1CCC(O)CC1)c1ccc(-c2c[nH]cn2)o1] |
| 105 | 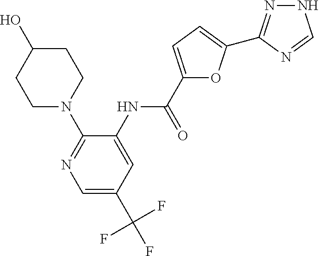 [SMILES: O=C(Nc1cc(C(F)(F)F)cnc1N1CCC(O)CC1)c1ccc(-c2nc[nH]n2)o1] |
| 106 | 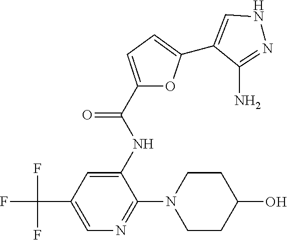 [SMILES: Nc1n[nH]cc1-c1ccc(C(=O)Nc2cc(C(F)(F)F)cnc2N2CCC(O)CC2)o1] |
| 107 | 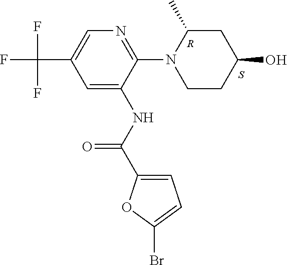 |
| 108 | 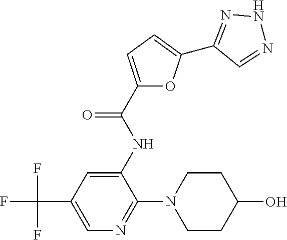 [SMILES: O=C(Nc1cc(C(F)(F)F)cnc1N1CCC(O)CC1)c1ccc(-c2cn[nH]n2)o1] |
| 109 | 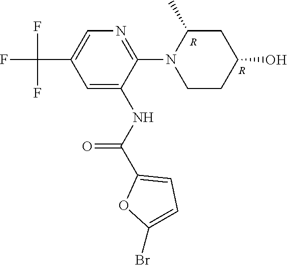 |
| 110 | 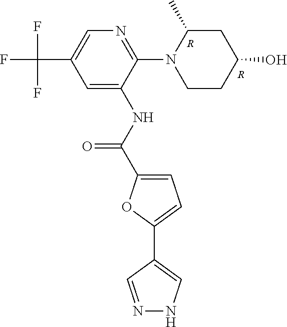 |
| 111 | 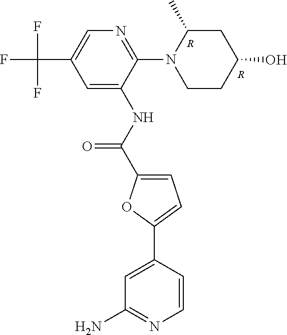 |
| 112 | 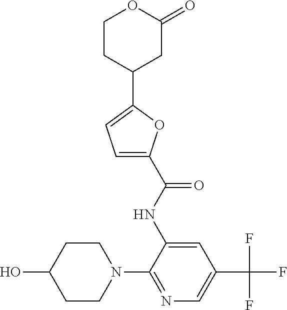 [SMILES: O=C1CC(c2ccc(C(=O)Nc3cc(C(F)(F)F)cnc3N3CCC(O)CC3)o2)CCO1] |
| 113 | 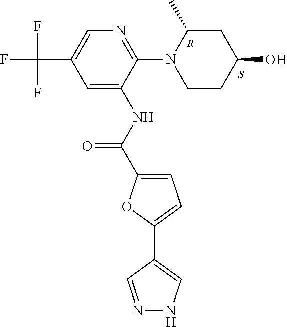 |
| 114 | 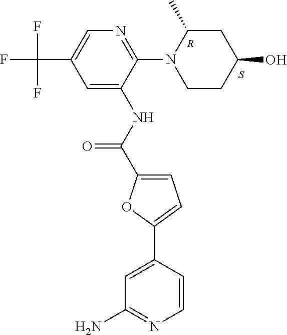 |
| 115 | 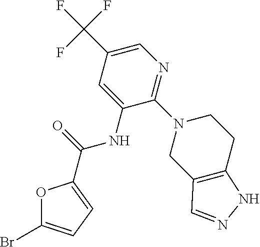 [SMILES: O=C(Nc1cc(C(F)(F)F)cnc1N1CCc2[nH]ncc2C1)c1ccc(Br)o1] |
| 116 | 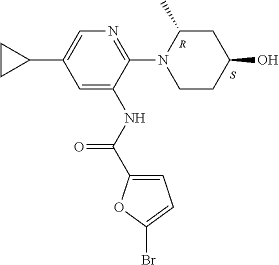 |
| 117 | 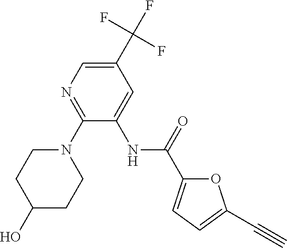 [SMILES: C#Cc1ccc(C(=O)Nc2cc(C(F)(F)F)cnc2N2CCC(O)CC2)o1] |
| 118 | 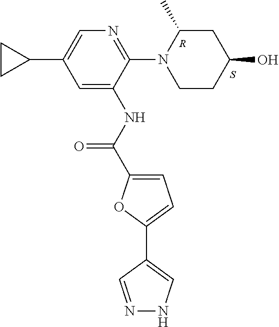 |
| 119 | 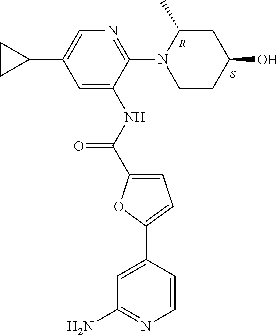 |
| 120 | 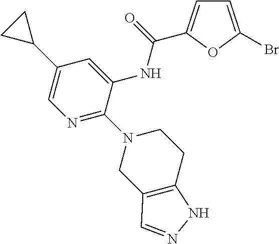 [SMILES: O=C(Nc1cc(C2CC2)cnc1N1CCc2[nH]ncc2C1)c1ccc(Br)o1] |
| 121 | 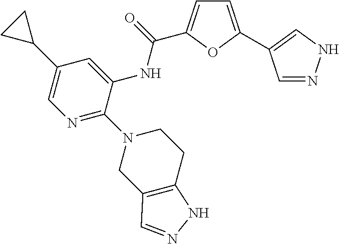 [SMILES: O=C(Nc1cc(C2CC2)cnc1N1CCc2[nH]ncc2C1)c1ccc(-c2cn[nH]c2)o1] |
| 122 | 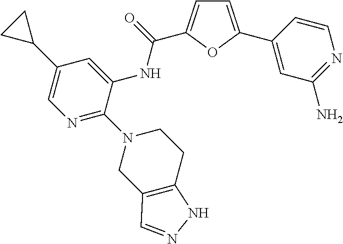 [SMILES: Nc1cc(-c2ccc(C(=O)Nc3cc(C4CC4)cnc3N3CCc4[nH]ncc4C3)o2)ccn1] |
| 123 | 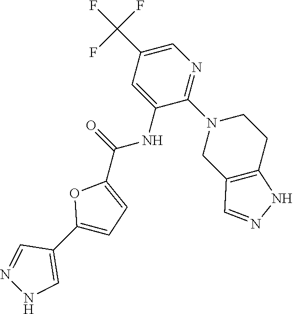 [SMILES: O=C(Nc1cc(C(F)(F)F)cnc1N1CCc2[nH]ncc2C1)c1ccc(-c2cn[nH]c2)o1] |
| 124 | 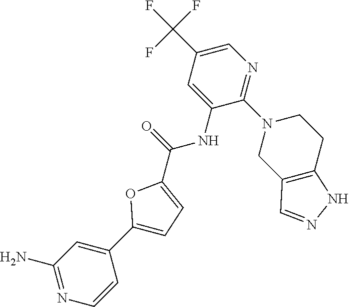 [SMILES: Nc1cc(-c2ccc(C(=O)Nc3cc(C(F)(F)F)cnc3N3CCc4[nH]ncc4C3)o2)ccn1] |
| 125 | 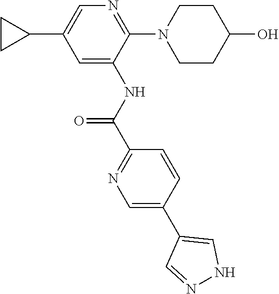 [SMILES: O=C(Nc1cc(C2CC2)cnc1N1CCC(O)CC1)c1ccc(-c2cn[nH]c2)cn1] |
| 126 | 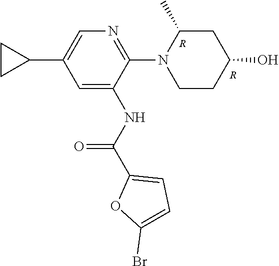 |
| 127 | 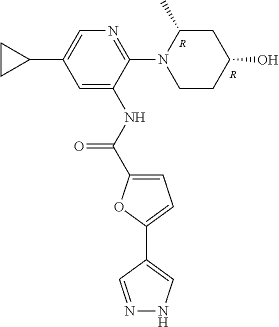 |
| 128 | 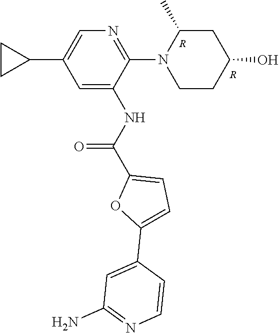 |
| 129 | 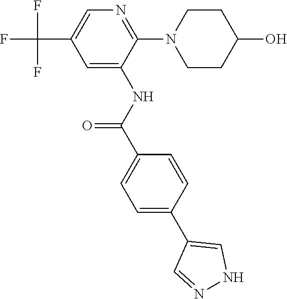 [SMILES: O=C(Nc1cc(C(F)(F)F)cnc1N1CCC(O)CC1)c1ccc(-c2cn[nH]c2)cc1] |
| 130 | 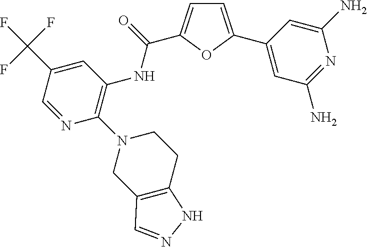 [SMILES: Nc1cc(-c2ccc(C(=O)Nc3cc(C(F)(F)F)cnc3N3CCc4[nH]ncc4C3)o2)cc(N)n1] |
| 131 | 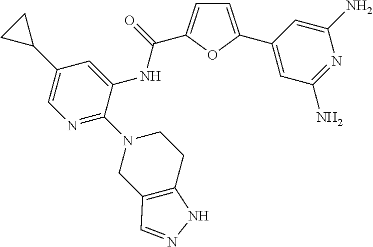 [SMILES: Nc1cc(-c2ccc(C(=O)Nc3cc(C4CC4)cnc3N3CCc4[nH]ncc4C3)o2)cc(N)n1] |
| 132 | 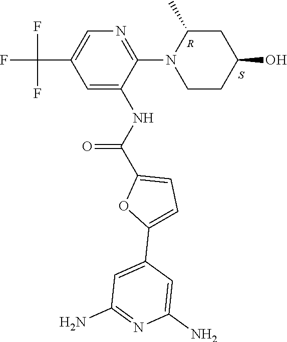 |
| 133 | 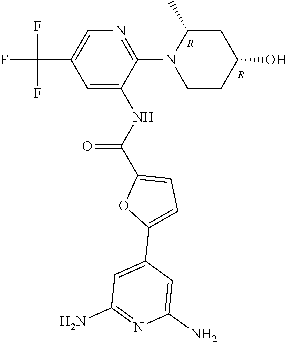 |
| 134 | 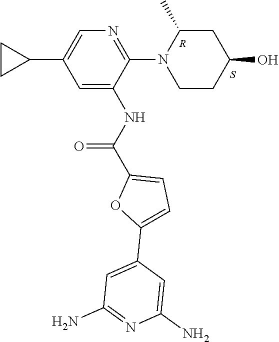 |
| 135 | 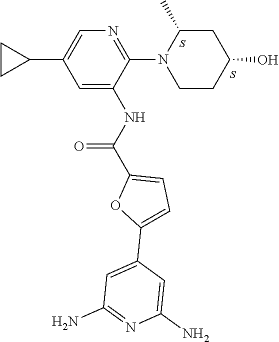 |
| 136 | 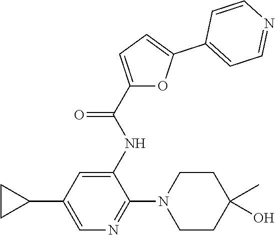 [SMILES: CC1(O)CCN(c2ncc(C3CC3)cc2NC(=O)c2ccc(-c3ccncc3)o2)CC1] |
| 137 | 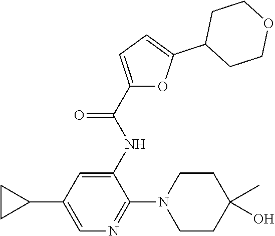 [SMILES: CC1(O)CCN(c2ncc(C3CC3)cc2NC(=O)c2ccc(C3CCOCC3)o2)CC1] |

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this disclosure, unless only one of the isomers is specifically indicated. Therefore, single stereochemical isomers as well as enantiomeric, diastereomeric, cis/trans, conformational, and rotational mixtures of the present compounds are within the scope of the disclosure. In some cases, the compounds disclosed herein are stereoisomers. “Stereoisomers” refer to compounds that differ in the chirality of one or more stereocenters. Stereoisomers include enantiomers and diastereomers. The compounds disclosed herein can exist as a single stereoisomer, or as a mixture of stereoisomers. Stereochemistry of the compounds shown herein indicate a relative stereochemistry, not absolute, unless discussed otherwise. As indicated herein, a single stereoisomer, diastereomer, or enantiomer refers to a compound that is at least more than 50% of the indicated stereoisomer, diastereomer, or enantiomer, and in some cases, at least 90% or 95% of the indicated stereoisomer, diastereomer, or enantiomer.

In some cases, the compounds of the disclosure are optically pure. As used herein, “optically pure” refers to the predominant presence of one enantiomer of a compound if multiple stereochemical configurations can exist (e.g., at least 99% enantiomeric excess).

Unless otherwise indicated, all tautomeric forms of the compounds of the disclosure are within the scope of the disclosure.

The compounds of the disclosure are defined herein by their chemical structures and/or chemical names. Where a compound is referred to by both a chemical structure and a chemical name, and the chemical structure and chemical name conflict, the chemical structure is determinative of the compound's identity.

As used herein, the term “alkyl” refers to straight chained and branched saturated hydrocarbon groups containing one to thirty carbon atoms, for example, one to twenty carbon atoms, or one to ten carbon atoms. The term Cₙ means the alkyl group has “n” carbon atoms. For example, C₆alkyl refers to an alkyl group that has 6 carbon atoms. C₁₋₇alkyl refers to an alkyl group having a number of carbon atoms encompassing the entire range (i.e., 1 to 6 carbon atoms), as well as all subgroups (e.g., 1-5, 2-5, 3-6, 1, 2, 3, 4, 5, and 6 carbon atoms). Nonlimiting examples of alkyl groups include, methyl, ethyl, n-propyl, isopropyl, n-butyl, sec-butyl(2-methylpropyl), and t-butyl (1,1-dimethylethyl). Unless otherwise indicated, an alkyl group can be an unsubstituted alkyl group or a substituted alkyl group.

As used herein, the term “alkylene” refers to a bivalent saturated aliphatic radical. The term Cₙ means the alkylene group has “n” carbon atoms, e.g., a C₁alkylene is CH₂. For example, C₁₋₆alkylene refers to an alkylene group having a number of carbon atoms encompassing the entire range, as well as all subgroups, as previously described for “alkyl” groups.

As used herein, the term “cycloalkyl” specifically refers to a non-aromatic ring in which each atom of the ring is carbon, i.e., a carbocycle, and can be monocyclic, bicyclic, bridged, fused or spirocyclic. The term Cₙ means the cycloalkyl group has “n” ring carbon atoms. For example, C₅ cycloalkyl refers to a cycloalkyl group that has 5 ring carbon atoms in the ring. C₃₋₈ cycloalkyl refers to cycloalkyl groups having a number of ring carbon atoms encompassing the entire range (i.e., 3 to 8 carbon atoms), as well as all subgroups (e.g., 4-8, 3-7, 4-7, 3-6, 4-6, 3-5, 4-5, 3, 4, 5, 6, 7, and 8 carbon atoms). Nonlimiting examples of cycloalkyl groups include cyclopropyl, cyclobutyl, cyclopentyl, cyclohexyl, cycloheptyl, and cyclooctyl.

As used herein, the term “heterocycle” refers to a ring which contains one to four heteroatoms independently selected from oxygen, nitrogen, and sulfur, and can be aromatic or non-aromatic (e.g., fully saturated or partially unsaturated). Additionally, heterocycles of the disclosure can be monocyclic, bicyclic, bridged, fused or spirocyclic. Nonlimiting examples of heterocycle groups include piperidine, piperazine, tetrahydrofuran, furan, tetrahydropyran, pyran, dihydrofuran, morpholine, oxazepane, oxazole, isoxazole, thiazole, pyrrole, and pyridine. Additional nonlimiting examples of heterocycle groups include benzothiazolyl, quinolyl, indole, isoquinolinyl, or quinazolinyl and the like.

As used herein, the term “alkoxy” refers to a “—O-alkyl” group.

As used herein, the term “halo” refers to a fluoro (F), chloro (Cl), bromo (Br), or iodo (I) group.

As used herein, the term “haloalkyl” refers to an alkyl group in which one or more of the hydrogen atoms are replaced by halogen. In some cases, a haloalkyl group is perhalogenated (i.e., all hydrogen atoms are replaced by halogen atoms). Haloalkyl groups include but are not limited to, chloromethyl, fluoromethyl, difluoromethyl, trifluoromethyl, 1,1-difluoroethyl, 2-fluoroethyl, 1-chloro-2-fluoromethyl and 2-fluoroisobutyl.

As used herein, the term “haloalkoxy” refers to an alkoxy, or “—O-alkyl” group in which one or more of the hydrogen atoms are replaced by a halo group. Such groups include but are not limited to, fluoromethoxy, chloromethoxy, bromomethoxy, fluoroethoxy, iodoethoxy and the like.

As used herein, the term “hydroxyalkyl” refers to an alkyl group in which one or more of the hydrogen atoms are replaced by a hydroxyl group (OH). Such groups include but are not limited to, hydroxymethyl, hydroxyethyl, and the like.

As used herein, a substituted” functional group is a functional, group having at least one hydrogen radical that is substituted with a non-hydrogen radical (i.e., a substituent). Examples of non-hydrogen radicals (or substituents) include, but are not limited to, alkyl, cycloalkyl, alkenyl, cycloalkyl, alkynyl, ether, aryl, heteroaryl, heterocycle, hydroxyl, oxy (or oxo), alkoxyl, ester, thioester, acyl, carboxyl, cyano, nitro, amino, sulfhydryl, and halo. When a substituted alkyl group includes more than one non-hydrogen radical, the substituents can be bound to the same carbon or different carbon atoms.

### Pharmaceutically Acceptable Salts

As used herein, the term “pharmaceutically acceptable salt” refers to salts of a compound which are, within the scope of sound medical judgment, suitable for use in contact with the tissues of humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, which is incorporated herein by reference. Pharmaceutically acceptable salts of the compounds described herein include those derived from suitable inorganic and organic acids and bases. These salts can be prepared in situ during the final isolation and purification of the compounds.

Where the compound described herein contains a basic group, or a sufficiently basic bioisostere, acid addition salts can be prepared by 1) reacting the purified compound in its free-base form with a suitable organic or inorganic acid and 2) isolating the salt thus formed. In practice, acid addition salts might be a more convenient form for use and use of the salt amounts to use of the free basic form.

Examples of pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Where the compound described herein contains a carboxyl group or a sufficiently acidic bioisostere, base addition salts can be prepared by 1) reacting the purified compound in its acid form with a suitable organic or inorganic base and 2) isolating the salt thus formed. In practice, use of the base addition salt might be more convenient and use of the salt form inherently amounts to use of the free acid form. Salts derived from appropriate bases include alkali metal (e.g., sodium, lithium, and potassium), alkaline earth metal (e.g., magnesium and calcium), ammonium and N⁺ (C₁₋₄alkyl) 4 salts. This disclosure also envisions the quaternization of any basic nitrogen-containing groups of the compounds disclosed herein. Water or oil-soluble or dispersible products may be obtained by such quaternization.

Basic addition salts include pharmaceutically acceptable metal and amine salts. Suitable metal salts include the sodium, potassium, calcium, barium, zinc, magnesium, and aluminum. The sodium and potassium salts are usually preferred. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which are frequently used in medicinal chemistry because of their low toxicity and acceptability for medical use. Ammonia, ethylenediamine, N-methyl-glucamine, lysine, arginine, ornithine, choline, N,N′-dibenzylethylenediamine, chloroprocaine, dietanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, dicyclohexylamine and the like.

Other acids and bases, although not in themselves pharmaceutically acceptable, may be employed in the preparation of salts useful as intermediates in obtaining the compounds described herein and their pharmaceutically acceptable acid or base addition salts.

It should be understood that a compound disclosed herein can be present as a mixture/combination of different pharmaceutically acceptable salts. Also contemplated are mixtures/combinations of compounds in free form and pharmaceutically acceptable salts.

### Pharmaceutical Formulations, Doses, and Routes of Administration

Also provided herein are pharmaceutical formulations that include an effective amount of compounds of the disclosure and one or more pharmaceutically acceptable excipients. As used herein, the term “formulation” is used interchangeable with “composition.”

An “effective amount” includes a “therapeutically effective amount” and a “prophylactically effective amount.” The term “therapeutically effective amount” refers to an amount effective in treating and/or ameliorating a disease or condition in a subject. The term “prophylactically effective amount” refers to an amount effective in preventing and/or substantially lessening the chances of a disease or condition in a subject. As used herein, the terms “patient” and “subject” may be used interchangeably and mean animals, such as dogs, cats, cows, horses, and sheep (i.e., non-human animals) and humans. Particular patients or subjects are mammals (e.g., humans). The terms “patient” and “subject” include males and females.

As used herein, the term “excipient” means any pharmaceutically acceptable additive, carrier, diluent, adjuvant, or other ingredient, other than the active pharmaceutical ingredient (API), suitably selected with respect to the intended form of administration, and consistent with conventional pharmaceutical practices.

The compounds of the disclosure can be administered alone or as part of a pharmaceutically acceptable composition or formulation. In addition, the compounds can be administered all at once, as for example, by a bolus injection, multiple times, e.g. by a series of tablets, or delivered substantially uniformly over a period of time, as for example, using transdermal delivery. It is also noted that the dose of the compound can be varied over time.

The compounds disclosed herein and other pharmaceutically active compounds, if desired, can be administered to a subject or patient by any suitable route, e.g. orally, topically, rectally, parenterally, (for example, subcutaneous injections, intravenous, intramuscular, intradermal, and intrathecal injection or infusion techniques), or as a buccal, inhalation, or nasal spray. The administration can be to provide a systemic effect (e.g. enteral or parenteral). All methods that can be used by those skilled in the art to administer a pharmaceutically active agent are contemplated. In some cases, the disclosed formulations can be administered orally or topically.

The compounds for use in the methods of the disclosure can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

The compounds of the disclosure can be administered to a subject or patient at dosage levels in the range of about 0.1 to about 3,000 mg per day. For a normal adult human having a body weight of about 70 kg, a dosage in the range of about 0.01 to about 100 mg per kilogram body weight is typically sufficient. The specific dosage and dosage range that will be used can potentially depend on a number of factors, including the requirements of the subject or patient, the severity of the condition or disease being treated, and the pharmacological activity of the compound being administered. The determination of dosage ranges and optimal dosages for a particular subject or patient is within the ordinary skill in the art.

### Methods of Treatment

The compounds disclosed herein, and pharmaceutically acceptable salts thereof, can act as modulators of SRPK1. Serine/arginine-rich splicing factor protein kinase-1 (SRPK1) is a kinase that plays an important role in both constitutive and alternative splicing processes by regulating intracellular localization of splicing factors. Several studies have further identified that inhibition/down-regulation of SRPK1 results in tumor-suppressive effects, such as reduced angiogenesis and reduced cancer cell migration. Additionally, knockdown of SRPK1 potently reduced vascular endothelial growth factor (VEGF) mediated angiogenesis in vivo in tumors and inhibition of SRPK1 reduced angiogenesis in vivo.

Thus, the disclosure provides a method of modulating serine/arginine-rich splicing factor protein kinase-1 (SRPK1) comprising contacting the SRPK1 with a therapeutically effective amount of a compound or salt disclosed herein or a formulation thereof, in an amount effective to modulate the SRPK1. In some cases, the contacting occurs in vitro. In some cases, the contacting occurs in vivo. In some cases, the contacting comprises administering to a subject in need thereof. As used herein, the terms “patient” and “subject” may be used interchangeably and mean animals, such as dogs, cats, cows, horses, and sheep (i.e., non-human animals) and humans. Particular patients are mammals (e.g., humans). In various cases, the subject suffers from a disease or disorder associated with aberrant SRPK1 activity. In some cases, the disease or disorder is cancer. In some cases, the cancer is colon cancer, breast cancer, prostate cancer, pancreatic cancer, kidney cancer, head and neck cancer, leukemia, lymphoma, liver cancer, brain cancer, ovarian cancer skin cancer, gastrointestinal cancer, or lung cancer. In various cases, the disease or disorder is vascular disease (e.g. vasoconstriction and disorders characterized by vasoconstriction, and cardiovascular disease), malignant or benign neoplasia (e.g. angiogenesis-dependent cancers, for example tumorous cancers), tumor metastasis, an inflammatory disorder, diabetes, diabetic retinopathy, diabetic neovascularization, diabetic macular edema, trachoma, retrolental hyperplasia, neovascular glaucoma, age-related macular degeneration, wet age-related macular degeneration (wAMD), macular edema, hemangioma, immune rejection of implanted corneal tissue, corneal angiogenesis associated with ocular injury or infection, Osier-Webber Syndrome, myocardial angiogenesis, wound granulation, telangiectasia, hemophiliac joints, angiofibroma, telangiectasia psoriasis scleroderma, pyogenic granuloma, rubeosis, obesity, arthritis (e.g. rheumatoid arthritis), hematopoiesis, vasculogenesis, gingivitis, atherosclerosis, endometriosis, neointimal hyperplasia, psoriasis, hirsutism, or proliferative retinopathy, idiopathic pulmonary fibrosis, or diabetic nephropathy.

In jurisdictions that forbid the patenting of methods that are practiced on the human body, the meaning of “administering” of a composition to a human subject or patient shall be restricted to prescribing a controlled substance that a human subject or patient will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### Synthesis of the Compounds of the Disclosure

The compounds of the disclosure can be synthesized by any method known in the art. For example, the compounds of the disclosure (compounds of Formula (I)) can be synthesized according to Schemes 1, 2, and 3.

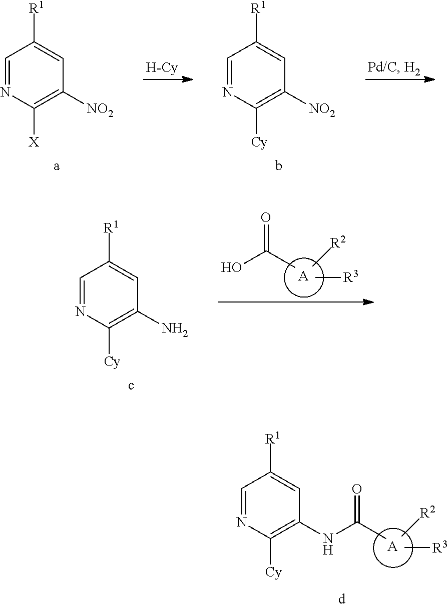

Coupling of a desired Cy group with a desired pyridyl moiety where X is halo (a), generates an intermediate (b), which is reduced with Pd/C and H₂ gas to generate an amino pyridyl moiety (c), which can be coupled with a desired ring A group to generate a compound of the disclosure (d).

An alternative synthetic scheme is shown below in Scheme 2.

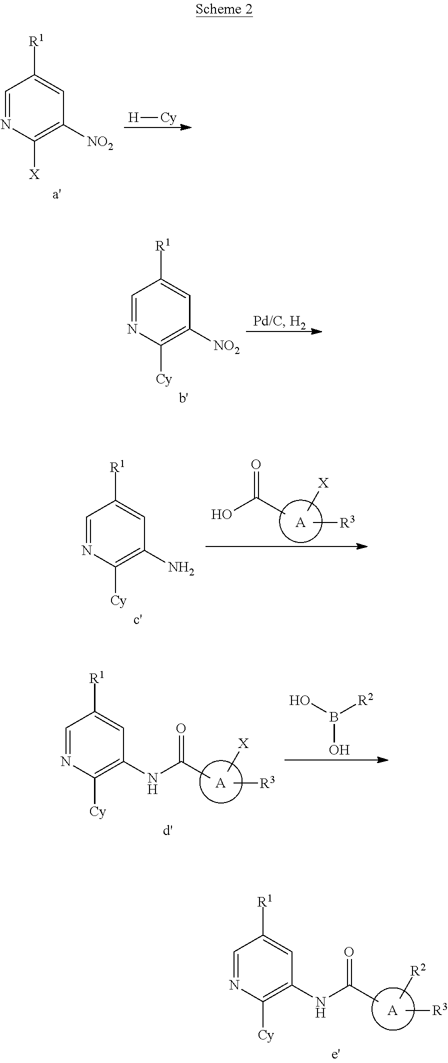

Coupling of a desired Cy group with a desired pyridyl moiety where X is halo (a′), generates an intermediate (b′), which can be reduced with Pd/C and H₂ gas to generate an amino pyridyl moiety (c′), which can be coupled with a desired halo-substituted ring A group to generate another intermediate (d), which can be coupled to a desired Het group that is conjugated to an organoboronic acid to generate a compound of the disclosure (e). It will be appreciated that coupling of a desired Het group can be performed using other coupling chemistry, including Kumada, Negishi, Stille, or Suzuki coupling conditions.

In some cases, the ring A moiety can be coupled to the pyridyl moiety prior to coupling the Cy group with the pyridyl moiety as shown in Scheme 3, below.

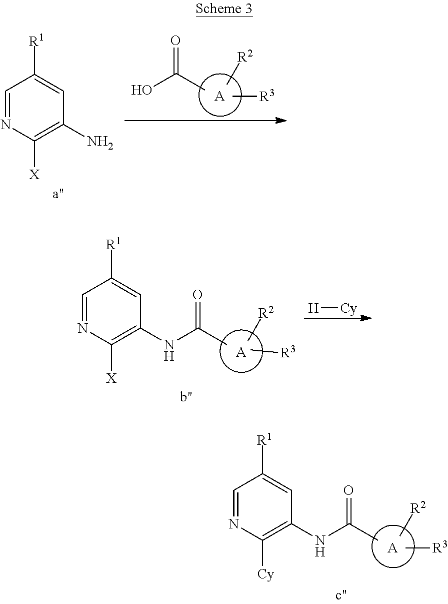

Coupling of a desired ring A group with a desired pyridyl moiety where X is halo (a″), generates an intermediate (b″), which can be coupled to a desired Cy group to generate a compound of the disclosure (c″).

### Embodiments of the Disclosure

1. A compound having a structure of Formula (I):

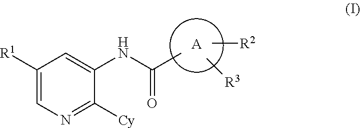

wherein
   ring A is a 5-10-membered heterocycle having 1, 2, or 3 ring heteroatoms independently selected from N, O, and S;
   Cy is a 4-10-membered heterocycle having 1, 2, or 3 ring heteroatoms independently selected from N, O, and S and optionally substituted with 1 to 4 R^{A};
   each R^{A} is independently halo, OH, oxo, C₁₋₆alkyl, C₁₋₆haloalkyl, C₁₋₆hydroxyalkyl, C₀₋₆alkylene-N(R^{N})₂, C₀₋₆alkylene-C(O)N(R^{N})₂, C₀₋₆alkylene-OC(O)C₁₋₆alkyl, C₀₋₆alkylene-C(O) C₁₋₆alkyl, C₀₋₆alkylene-CO₂R^{N}, or C₀₋₃alkylene-4-8-membered heterocycle having 1, 2, 3 or 3 ring heteroatoms independently selected from N, O, and S;
   R¹ is H, halo, C₁₋₆alkyl, C₁₋₆haloalkyl, C₁₋₆alkoxy, C₁₋₆haloalkoxy, or C₃₋₆cycloalkyl;
   R² is halo, C₁₋₆alkoxy, C₁₋₆haloalkoxy, C₁₋₆alkyl, C₂₋₆alkene, C₂₋₆alkyne, C₁₋₆haloalkyl, CO₂H, or Het;
   Het is a 5-8-membered heterocycle having 1, 2, or 3 ring heteroatoms independently selected from N, O, and S, and is optionally substituted with 1 or 2 R^{B};
   each R^{B} is independently halo, OH, oxo (═O), C₁₋₆alkyl, C₁₋₆haloalkyl, C₁₋₆alkoxy, C₁₋₆haloalkoxy, C₀₋₆alkylene-N(R^{N})₂, or C₀₋₆alkylene-4-8-membered heterocycle having 1, 2, 3 or 3 ring heteroatoms independently selected from N, O, and S;
   R³ is H, halo, or C₁₋₆alkyl; and
   each R^{N} is independently H or C₁₋₃alkyl, or a pharmaceutically acceptable salt thereof.
2. The compound or salt of embodiment 1, wherein ring A is aromatic, optionally a 5- or 6-memebred ring.
3. The compound or salt of embodiment 1, wherein ring A is furan, oxazole, isoxazole, thiophene, thiazole, imidazole, pyrazole, triazole, pyridine, pyrimidine, pyridazine, or pyrazine.
4. The compound or salt of embodiment 3, wherein ring A is furan.
5. The compound or salt of embodiment 4, wherein ring A is 2-furan.
6. The compound or salt of embodiment 5, having a structure of Formula (II):

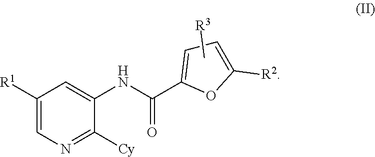

7. The compound or salt of embodiment 3, wherein ring A is pyridine.
8. The compound or salt of embodiment 7, having a structure of Formula (III):

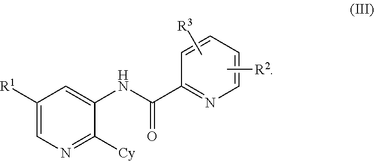

9. The compound or salt of any one of embodiments 1 to 8, wherein Cy is azetidine, pyrrolidine, piperidine, piperazine, azepane, morpholine, thiomorpholine, pyrazole, pyridine, pyrazine, pyrimidine, pyridazine, 2,4,6,7-tetrahydro-pyrazolo[4,3-c]pyridine, 2-oxa-7-azaspiro[3.5]nonane, 7-azaspiro[3.5]nonane, 2-oxa-8-azaspiro[4.5]decane, 2,8-diazaspiro[4.5]decane, or 1,8-diazaspiro[4.5]decane.
10. The compound or salt of any one of embodiments 1 to 9, wherein Cy is unsubstituted.
11. The compound or salt of any one of embodiments 1 to 9, wherein Cy is substituted with 1 R^{A}.
12. The compound or salt of any one of embodiments 1 to 9, wherein Cy is substituted with 2 R^{A}.
13. The compound or salt of embodiment 11 or 12, wherein at least one R^{A} is OH, F, CH₃, C(O)N(R^{N})₂, CH₂OH, oxo, CF₃, OC(O)CH₃, CO₂CH₃, CO₂H, CH₂-pyridine, C(O)CH₃, or CH₂N(R^{N})₂.
14. The compound or salt of any one of embodiments 1 to 8, wherein Cy is

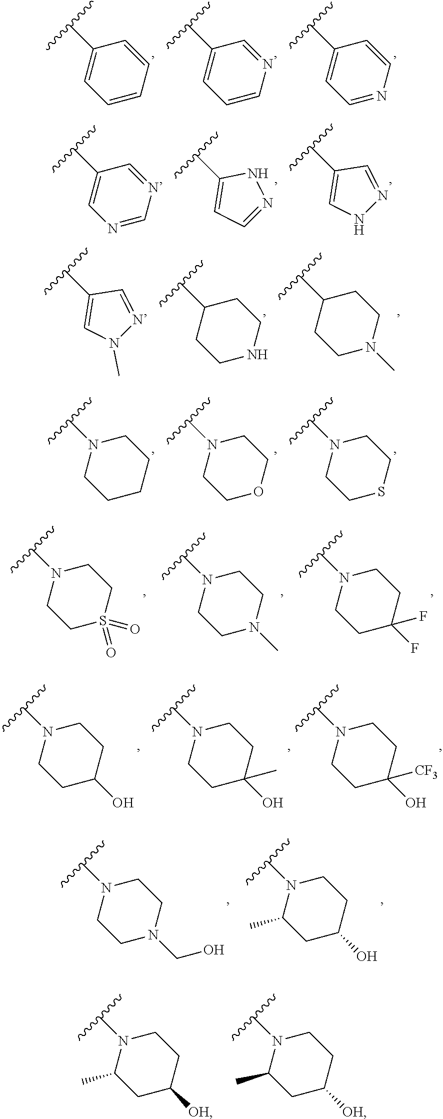

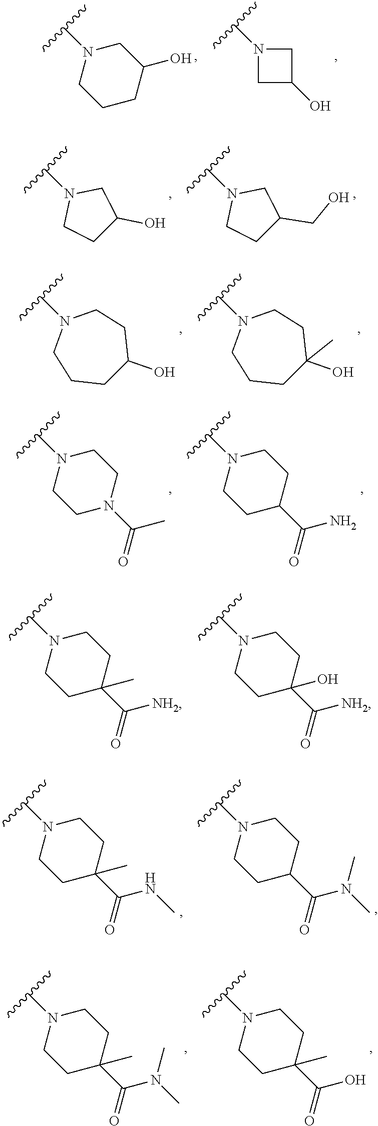

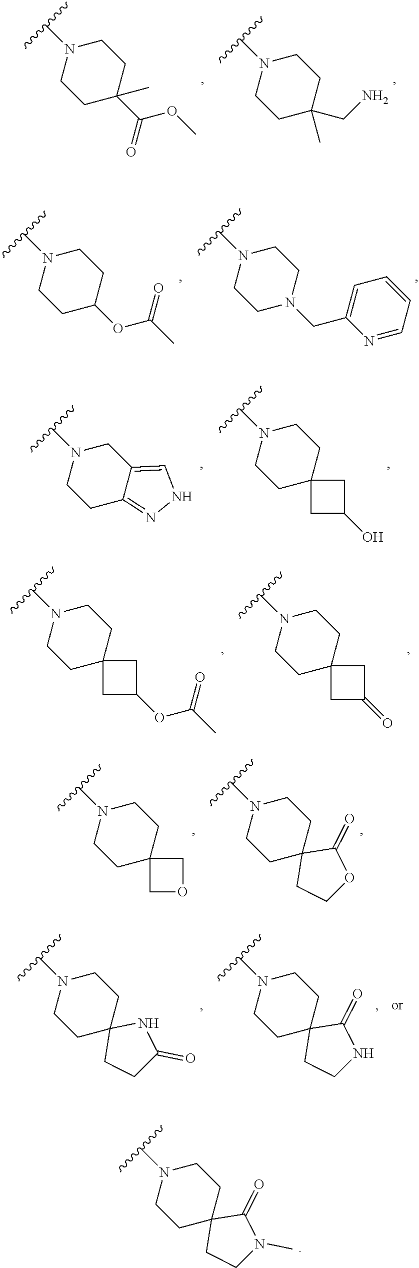

15. The compound or salt of any one of embodiments 1 to 14, wherein R¹ is halo.
16. The compound or salt of any one of embodiments 1 to 14, wherein R¹ is CF₃.
17. The compound or salt of any one of embodiments 1 to 14, wherein R¹ is cyclopropyl.
18. The compound or salt of any one of embodiments 1 to 17, wherein R² is halo, C₁₋₃alkoxy, or C₁₋₃haloalkyl.
19. The compound or salt of any one of embodiments 1 to 17, wherein R² is Het.
20. The compound or salt of embodiment 19, wherein Het is pyridine, pyrazole, tetrahydropyran, pyrazine, pyrimidine, pyridazine, or piperidine.
21. The compound or salt of embodiment 19 or 20, wherein Het is unsubstituted.
22. The compound or salt of embodiment 19 or 20, wherein Het is substituted with 1 R^{B}.
23. The compound or salt of embodiment 19 or 20, wherein Het is substituted with 2 R^{B}.
24. The compound or salt of embodiment 22 or 23, wherein at least one R^{B} is NH₂, OH, CH₃, or F.
25. The compound or salt of any one of embodiments 1 to 17, wherein Het is

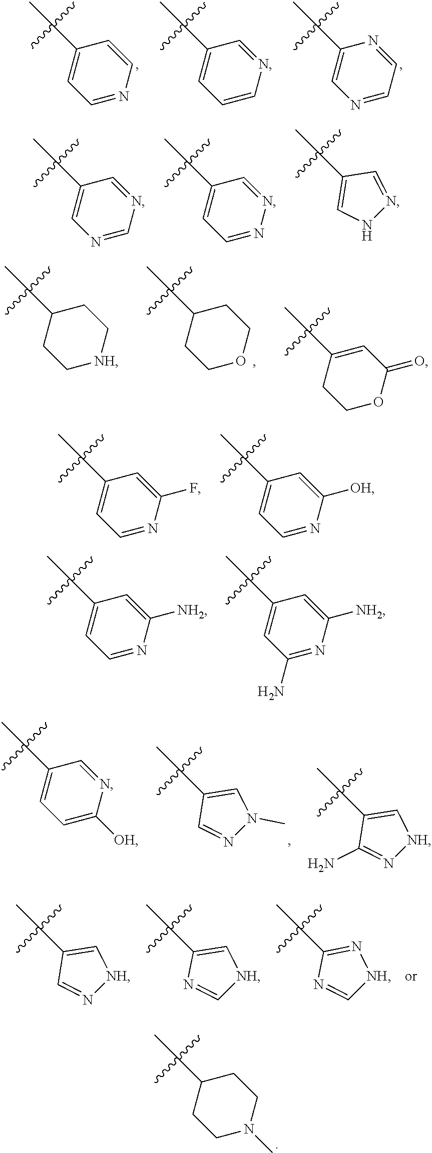

26. The compound or salt of any one of embodiments 1 to 25, wherein R³ is H.
27. A compound as listed in Table A, or a pharmaceutically acceptable salt thereof.
28. A pharmaceutical composition comprising the compound or salt of any one of embodiments 1 to 27 and a pharmaceutically acceptable excipient.
29. A method of inhibiting SRPK1 comprising contacting SRPK1 with an effective amount of the compound or salt of any one of embodiments 1 to 27 to inhibit SRPK1.
30. Use of the compound or salt of any one of embodiments 1 to 27 as a SRPK1 inhibitor.
31. The compound or salt of any one of embodiments 1 to 27 for use as a medicament.
32. A method of treating a subject suffering from a disease or disorder associated with aberrant SRPK1 activity comprising administering to the subject a therapeutically effective amount of the compound or salt of any one of embodiments 1 to 27.
33. The method of embodiment 32, wherein the disease or disorder is vascular disease (e.g. vasoconstriction and disorders characterized by vasoconstriction, and cardiovascular disease), malignant or benign neoplasia (e.g. angiogenesis-dependent cancers, for example tumorous cancers), tumor metastasis, an inflammatory disorder, diabetes, diabetic retinopathy, diabetic neovascularization, diabetic macular edema, trachoma, retrolental hyperplasia, neovascular glaucoma, age-related macular degeneration, wet age-related macular degeneration (wAMD), macular edema, hemangioma, immune rejection of implanted corneal tissue, corneal angiogenesis associated with ocular injury or infection, Osier-Webber Syndrome, myocardial angiogenesis, wound granulation, telangiectasia, hemophiliac joints, angiofibroma, telangiectasia psoriasis scleroderma, pyogenic granuloma, rubeosis, obesity, arthritis (e.g. rheumatoid arthritis), hematopoiesis, vasculogenesis, gingivitis, atherosclerosis, endometriosis, neointimal hyperplasia, psoriasis, hirsutism, proliferative retinopathy, idiopathic pulmonary fibrosis, or diabetic nephropathy.
34. The method of embodiment 32, wherein the disease or disorder is cancer.
35. The method of embodiment 34, wherein the cancer is colon cancer, breast cancer, prostate cancer, pancreatic cancer, kidney cancer, head and neck cancer, leukemia, lymphoma, liver cancer, brain cancer, ovarian cancer skin cancer, gastrointestinal cancer, or lung cancer.

### EXAMPLES

The following examples are provided for illustration and are not intended to limit the scope of the invention.

### Synthesis of Compounds

LCMS method 1 was performed with the following materials and parameters: Waters SunFire C18 50*4.6 mm 5 μm 2.000 ml/min 2.6 min Column Temperature: 40° C. Gradient: 5% B hold for 0.2 min, increase to 95% B within 1.40 min, hold at 95% B for 0.9 min, then back to 5% B within 0.01 min. Pump A: 0.03% TFA in H₂O; Pump B: 0.03% TFA in ACN. Compounds 001-023, 098, and 099 were analyzed using LCMS Method 1.

LCMS method 2 was performed with the following materials and parameters: Shimadzu Acquity UPLC method; Mass-spectrometer: Shimadzu LCMS-2020; Column: Shim-pack Scepter C₁₈₋₁₂₀ (3.0×33 mm) 3 μm; Solvent A: 0.05% HCOOH in water; Solvent B: 0.05% HCOOH in acetonitrile; Gradient: 3 min total (time (min)/% B): 0/5, 1.3/95, 2.0/95, 2.1/5, 3/5; Flow rate: 1.3 mL/min wave length: 254 nm. Compounds 024-097 and 100-105 were analyzed using LCMS Method 2.

### Example 1—Preparation of N-(2-(4-(pyridin-2-ylmethyl) piperazin-1-yl)-5-(trifluoromethyl)pyridin-3-yl)-5-(tetrahydro-2H-pyran-4-yl) furan-2-carboxamide (Compound 002)

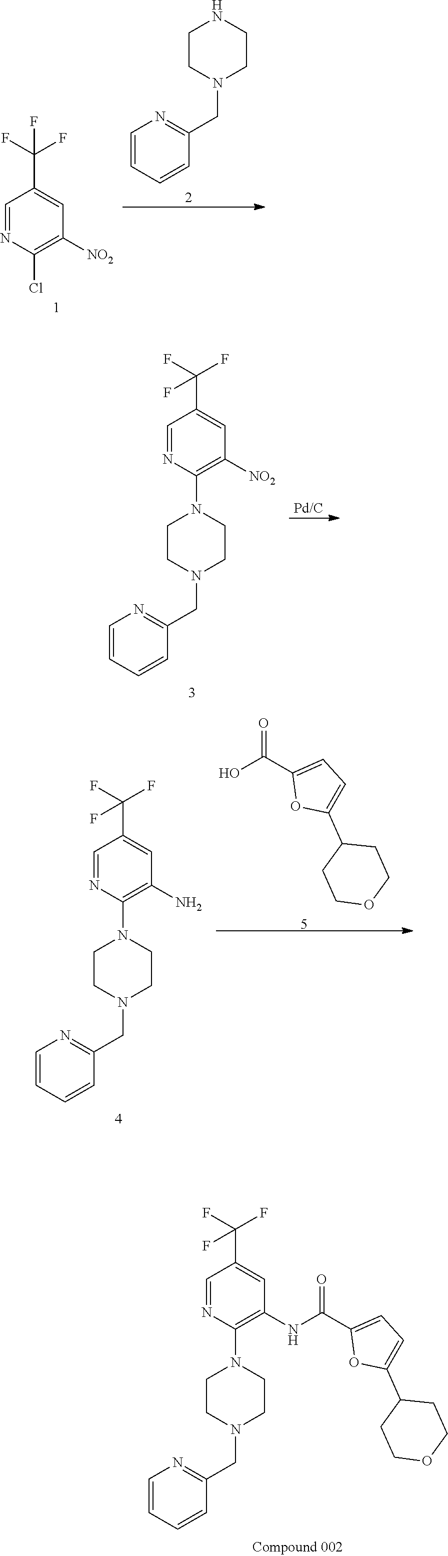 [SMILES: O=[N+]([O-])c1cc(C(F)(F)F)cnc1Cl ; c1ccc(CN2CCNCC2)nc1 ; O=[N+]([O-])c1cc(C(F)(F)F)cnc1N1CCN(Cc2ccccn2)CC1 ; Nc1cc(C(F)(F)F)cnc1N1CCN(Cc2ccccn2)CC1 ; O=C(O)c1ccc(C2CCOCC2)o1 ; O=C(Nc1cc(C(F)(F)F)cnc1N1CCN(Cc2ccccn2)CC1)c1ccc(C2CCOCC2)o1]

Step-1. To a solution of 2-chloro-3-nitro-5-(trifluoromethyl)pyridine (2.0 g, 8.8 mmol) and 1-(pyridin-2-ylmethyl) piperazine (3.13 g, 17.6 mmol) in DMF (30 mL) was added K₂CO₃ (2.4 g, 17.6 mmol). The reaction mixture was stirred overnight at 80° C. LCMS showed the reaction was completed. The reaction mixture was poured on to water (10 ml) and extracted with EA (3×6 ml), brine (6 ml), which was dried over anhydrous Na₂SO₄, filtered, concentrated in vacuum to give the desired product 1-methyl-4-(3-nitro-5-(trifluoromethyl)pyridin-2-yl) piperazine. LCMS: 368 [M+1]; Retention time: 1.15 min.

Step-2. To a solution of 1-methyl-4-(3-nitro-5-(trifluoromethyl)pyridin-2-yl) piperazine (4 g, 10.9 mmol) and Pd/C (400 mg, 3.63 mmol) in MeOH (50 mL) was stirred under 1 atm of H₂ at room temperature for 4 h. LCMS showed the reaction was completed. The reaction mixture was filtered with diatomaceous earth, and the filtrate was concentrated to give the desired product 2-(4-methylpiperazin-1-yl)-5-(trifluoromethyl)pyridin-3-amine. LCMS: 338 [M+1]; Retention time: 1.52 min.

Step-3. To a solution of 2-(4-methylpiperazin-1-yl)-5-(trifluoromethyl)pyridin-3-amine (100 mg, 0.3 mmol), 5-(tetrahydro-2H-pyran-4-yl) furan-2-carboxylic acid (58 mg, 0.3 mmol) and Pyridine (0.13 mL, 0.9 mmol) in DCM (5 mL) cooled to 0° C. was added POCl₃ (0.1 mL, 1.5 mmol). The reaction mixture was stirred overnight at room temperature. LCMS showed the reaction was completed. The reaction mixture was poured on to water (5 ml) and extracted with EA (3×3 ml), brine (6 ml), which was dried over anhydrous Na₂SO₄, filtered, and evaporated to give crude product. The crude product was purified by prep-HPLC (TFA) to give the desired product N-(2-(4-(pyridin-2-ylmethyl) piperazin-1-yl)-5-(trifluoromethyl)pyridin-3-yl)-5-(tetrahydro-2H-pyran-4-yl) furan-2-carboxamide. LCMS Method 1:516 [M+1]; Retention time: 1.31 min.

¹H NMR (400 MHz, CDCl₃) δ 8.89 (s, 1H), 8.66 (s, 1H), 8.51 (d, J=4.3 Hz, 1H), 8.28 (s, 1H), 7.63 (t, J=7.3 Hz, 1H), 7.42 (d, J=7.7 Hz, 1H), 7.14 (d, J=3.4 Hz, 2H), 6.18 (d, J=3.2 Hz, 1H), 4.04 (s, 2H), 3.71 (s, 2H), 3.54 (t, J=10.9 Hz, 2H), 3.19 (s, 4H), 2.95 (d, J=11.2 Hz, 1H), 2.74 (s, 4H), 1.98 (s, 4H).

The following compounds were prepared in a similar manner to the procedure described above.

| | LCMS | |
|---|---|---|
| Compound | (ESI): | |
| No. | [M + H]⁺ | ¹H NMR |
| 005 | 440.0 | ¹H NMR (400 MHz, CDCl₃) δ 8.95-8.84 (m, 2H), 8.29-8.22 |
| | | (m, 1H), 7.14-7.08 (m, 1H), 6.23-6.14 (m, 1H), 4.11-3.96 |
| | | (m, 2H), 3.54-3.41 (m, 2H), 3.30-3.21 (m, 2H), 3.02-2.86 |
| | | (m, 3H), 2.20-2.05 (m, 4H), 1.94-1.84 (m, 5H). |
| 007 | 406.0 | ¹H NMR (400 MHz, DMSO-d₆) δ 9.21 (s, 1H), 8.34 (d, J = 2.4 |
| | | Hz, 1H), 8.11 (d, J = 2.4 Hz, 1H), 7.28 (d, J = 3.4 Hz, 1H), 6.43 |
| | | (d, J = 3.3 Hz, 1H), 4.74 (d, J = 3.6 Hz, 1H), 3.91 (d, J = 10.2 Hz, |
| | | 2H), 3.67 (dd, J = 8.0, 4.2 Hz, 1H), 3.46 (dd, J = 11.6, 10.0 Hz, |
| | | 2H), 3.26 (d, J = 12.8 Hz, 2H), 3.26 (d, J = 12.8 Hz, 2H), 3.02 |
| | | (dd, J = 9.4, 5.7 Hz, 1H), 2.83 (t, J = 10.0 Hz, 2H), 2.03-1.81 |
| | | (m, 5H), 1.65 (ddt, J = 27.7, 18.5, 6.7 Hz, 4H). |
| 008 | 417.0 | ¹H NMR (400 MHz, DMSO-d₆) δ 9.83 (s, 1H), 8.93 (d, J = 1.1 |
| | | Hz, 1H), 8.53 (d, J = 1.8 Hz, 1H), 7.74 (dd, J = 7.4, 2.0 Hz, 2H), |
| | | 7.55-7.42 (m, 3H), 7.17 (d, J = 3.5 Hz, 1H), 6.36 (d, J = 3.4 Hz, |
| | | 1H), 3.89 (dd, J = 7.8, 2.3 Hz, 2H), 3.42 (td, J = 11.5, 1.9 Hz, |
| | | 2H), 2.94 (s, 1H), 1.83 (dd, J = 12.9, 1.9 Hz, 2H), 1.62 (td, J = |
| | | 11.8, 3.6 Hz, 2H). |
| 009 | 418.0 | ¹H NMR (400 MHz, DMSO-d₆) δ 10.27 (s, 1H), 9.16-8.92 (m, |
| | | 2H), 8.50 (d, J = 1.3 Hz, 1H), 8.36 (d, J = 7.9 Hz, 1H), 7.86 (d, J = |
| | | 3.5 Hz, 1H), 7.74 (s, 1H), 7.18 (d, J = 3.4 Hz, 1H), 6.37 (d, J = |
| | | 3.2 Hz, 1H), 3.90 (d, J = 11.5 Hz, 2H), 3.44 (dd, J = 11.6, 9.9 Hz, |
| | | 2H), 3.01-2.89 (m, 1H), 1.87 (d, J = 11.2 Hz, 2H), 1.70-1.52 |
| | | (m, 2H). |
| 010 | 418.0 | ¹H NMR (400 MHz, DMSO-d₆) δ 10.26 (s, 1H), 9.07-8.94 (m, |
| | | 1H), 8.81-8.66 (m, 2H), 8.57-8.43 (m, 1H), 7.84 (d, J = 5.6 |
| | | Hz, 2H), 7.26-7.09 (m, 1H), 6.41-6.28 (m, 1H), 3.91 (s, 2H), |
| | | 3.88 (s, 2H), 3.46-3.39 (m, 2H), 1.91-1.75 (m, 2H), 1.68- |
| | | 1.55 (m, 2H). |
| 011 | 421.0 | ¹H NMR (400 MHz, DMSO-d₆) δ 10.15 (s, 1H), 8.82 (s, 1H), |
| | | 8.31 (s, 1H), 8.27 (d, J = 1.5 Hz, 1H), 7.30 (d, J = 3.4 Hz, 1H), |
| | | 6.43 (d, J = 3.3 Hz, 1H), 3.90 (s, 3H), 3.50-3.46 (m, 2H), 3.43 |
| | | (d, J = 1.8 Hz, 2H), 3.03 (s, 1H), 1.93 (d, J = 11.3 Hz, 2H), 1.72 |
| | | (dd, J = 12.4, 3.6 Hz, 2H). |
| 012 | 407.0 | ¹H NMR (400 MHz, DMSO-d₆) δ 12.88 (s, 1H), 9.43 (d, J = 2.0 |
| | | Hz, 1H), 8.85 (d, J = 2.6 Hz, 1H), 8.63 (d, J = 1.1 Hz, 1H), 8.08 |
| | | (d, J = 1.4 Hz, 1H), 7.27 (d, J = 3.5 Hz, 1H), 6.89-6.69 (m, 1H), |
| | | 6.48 (d, J = 3.4 Hz, 1H), 3.97 (dd, J = 7.6, 2.4 Hz, 2H), 3.52 (td, |
| | | J = 11.5, 2.0 Hz, 3H), 3.12 (s, 1H), 1.96 (dd, J = 12.8, 2.1 Hz, |
| | | 2H), 1.76 (dd, J = 12.5, 3.6 Hz, 2H). |
| 013 | 419.0 | ¹H NMR (400 MHz, DMSO-d₆) δ 10.35 (s, 1H), 9.25 (s, 1H), |
| | | 9.10 (s, 2H), 9.07-9.02 (m, 1H), 8.50 (d, J = 1.6 Hz, 1H), 7.19 |
| | | (d, J = 3.5 Hz, 1H), 6.38 (dd, J = 3.5, 0.8 Hz, 1H), 3.98-3.83 |
| | | (m, 2H), 3.43 (td, J = 11.6, 2.1 Hz, 2H), 2.97 (tt, J = 11.2, 3.7 Hz, |
| | | 1H), 1.87 (dd, J = 12.8, 2.0 Hz, 2H), 1.73-1.56 (m, 2H). |
| 014 | 400.0 | ¹H NMR (400 MHz, CDCl₃) δ 13.31 (s, 1H), 9.58 (s, 1H), 8.89 |
| | | (s, 2H), 8.85 (d, J = 2.7 Hz, 1H), 8.48 (s, 1H), 8.10 (s, 2H), 7.93 |
| | | (s, 1H), 7.47 (d, J = 3.7 Hz, 1H), 7.35 (s, 1H), 6.67 (s, 1H) |
| 015 | 448.0 | ¹H NMR (400 MHz, DMSO-d₆) δ 10.15-10.02 (m, 1H), 8.51- |
| | | 8.37 (m, 1H), 8.11-8.09 (m, 1H), 8.06-8.02 (m, 1H), 7.88- |
| | | 7.74 (m, 1H), 7.62-7.55 (m, 2H), 7.33-7.24 (m, 2H), 3.73- |
| | | 3.67 (m, 2H), 3.11-3.02 (m, 2H), 2.02-1.95 (m, 1H), 1.84- |
| | | 1.75 (m, 2H), 1.53-1.42 (m, 2H) |
| 016 | 451.0 | ¹H NMR (400 MHz, DMSO-d₆) δ 8.45 (s, 1H), 8.35 (s, 1H), 8.08 |
| | | (d, J = 1.9 Hz, 1H), 7.89 (d, J = 4.7 Hz, 1H), 7.77 (s, 1H), 7.60 |
| | | (d, J = 3.6 Hz, 1H), 7.48 (d, J = 3.6 Hz, 1H), 3.68 (d, J = 4.3 Hz, |
| | | 1H), 3.12-3.00 (m, 4H), 1.80 (d, J = 9.6 Hz, 2H), 1.54-1.42 |
| | | (m, 2 H) |
| 024 | 413.1 | ¹H NMR (400 MHz, DMSO-d₆) δ 8.63 (dd, J = 4.0, 1.6 Hz, 2H), |
| | | 8.43 (d, J = 2.4 Hz, 1H), 7.82-7.74 (m, 3H), 7.37 (d, J = 4.0 Hz, |
| | | 1H), 7.14 (d, J = 4.0 Hz, 1H), 4.24 (s, 1H), 3.45 (d, J = 12.0 Hz, |
| | | 2H), 3.29-3.20 (m, 2H), 1.71-1.56 (m, 4H), 1.17 (s, 3H). |
| 025 | 420.1 | ¹H NMR (400 MHz, DMSO-d₆) δ 9.14 (s, 1H), 8.42 (d, J = 2.4 |
| | | Hz, 1H), 8.11 (d, J = 2.4 Hz, 1H), 7.27 (d, J = 4.0 Hz, 1H), 6.44 |
| | | (dd, J = 4.0, 1.2 Hz, 1H), 4.34 (s, 1H), 3.92 (ddd, J = 12.0, 4.0, |
| | | 2.4 Hz, 2H), 3.45 (td, J = 12.0, 2.4 Hz, 2H), 3.65-3.45 (m, 4H), |
| | | 3.19-2.90 (m, 3H), 2.01-1.48 (m, 6H), 1.19 (s, 3H). |
| 026 | 447.1 | ¹H NMR (400 MHz, DMSO-d₆) δ 9.94 (s, 1H), 8.70-8.68 (d, J = |
| | | 8.0 Hz, 2H), 8.44-8.43 (d, J = 4.0 Hz, 1H), 8.19-8.18 (d, J = 4.0 |
| | | Hz, 1H), 7.90-7.89 (d, J = 4.0 Hz, 2H), 7.51-7.47 (dd, J = 4.0, |
| | | 12.0 Hz, 2H), 4.35 (s, 1H), 3.35-3.30 (m, 4H), 1.66-1.54 (m, |
| | | 4H), 1.17 (s, 3H). |
| 027 | 454.1 | ¹H NMR (400 MHz, DMSO-d₆) δ 9.33 (s, 1H), 8.41 (d, J = 4.0 |
| | | Hz, 1H), 8.35 (d, J = 4.0 Hz, 1H), 7.27 (d, J = 4.0 Hz, 1H), 6.42 |
| | | (d, J = 4.0 Hz, 2H), 4.38 (s, 1H), 3.93-3.89 (m, 2H), 3.47-3.41 |
| | | (m, 3H), 3.26-3.19 (m, 2H), 3.04-2.98 (m, 1H), 1.92 (d, J = 4.0 |
| | | Hz, 2H), 1.89-1.57 (m, 6H), 1.17 (s, 3H). |
| 028 | 454.2 | ¹H NMR (400 MHz, DMSO-d₆) δ 9.15 (s, 1H), 8.02 (d, J = 2.4 |
| | | Hz, 1H), 7.89 (d, J = 2.4 Hz, 1H), 7.19 (d, J = 3.6 Hz, 1H), 6.39 |
| | | (dd, J = 3.6, 0.8 Hz, 1H), 4.86-4.76 (m, 1H), 3.93-3.87 (m, |
| | | 2H), 3.47-3.38 (m, 2H), 3.18-3.08 (m, 2H), 3.06-2.96 (m, |
| | | 1H), 2.90-2.81 (m, 2H), 2.02-1.94 (m, 5H), 1.93-1.85 (m, |
| | | 3H), 1.74-1.68 (m, 4H), 0.97-0.91 (m, 2H), 0.65-0.58 (m, |
| | | 2H). |
| 029 | 412.1 | ¹H NMR (400 MHz, DMSO-d₆) δ 9.15 (s, 1H), 8.02 (d, J = 2.4 |
| | | Hz, 1H), 7.89 (d, J = 2.4 Hz, 1H), 7.20 (d, J = 3.6 Hz, 1H), 6.39 |
| | | (dd, J = 3.2, 1.2 Hz, 1H), 4.86-4.76 (m, 1H), 3.94-3.87 (m, |
| | | 2H), 3.47-3.39 (m, 2H), 3.18-3.09 (m,, 2H), 3.07-2.95 (m, |
| | | 1H), 2.91-2.82 (m, 2H), 1.99-1.94 (m, 2H), 1.93-1.86 (m, |
| | | 3H), 1.79-1.59 (m, 5H), 0.99-0.90 (m, 2H), 0.66-0.59 (m, |
| | | 2H). |
| 031 | 454.1 | ¹H NMR (400 MHz, DMSO-d₆) δ 9.83 (s, 1H), 8.45-8.27 (m, |
| | | 1H), 7.74 (d, J = 4.0 Hz, 1H), 7.19 (d, J = 4.0 Hz, 1H), 6.36 (dd, |
| | | J = 4.0 Hz, 1H), 4.50 (d, J = 4.0 Hz, 1H), 4.05-3.38 (m, 12H), |
| | | 3.01-2.94 (m, 2H), 2.05-1.77 (m, 2H), 1.73-1.43 (m, 4H). |
| 032 | 496.1 | ¹H NMR (400 MHz, DMSO-d₆) δ 9.34 (s, 1H), 8.39-8.38 (m, 1H), 8.34 |
| | | (d, J = 2.4 Hz, 1H), 7.25 (d, J = 3.6 Hz, 1H), 6.4 (dd, J = |
| | | 3.6, 0.8 Hz, 1H), 3.91-3.86 (m, 2H), 3.62 (s, 3H), 3.45-3.36 |
| | | (m, 4H), 3.02-2.95 (m, 3H), 2.11-2.07 (m, 2H), 1.90-1.86 (m, |
| | | 2H), 1.72-1.54 (m, 4H), 1.17 (s, 3H). |
| 033 | 447.1 | ¹H NMR (400 MHz, DMSO-d₆) δ 9.57 (s, 1H), 8.66 (dd, J = 4.4, |
| | | 1.6 Hz, 2H), 7.93 (d, J = 2.4 Hz, 1H), 7.87 (d, J = 2.4 Hz, 1H), |
| | | 7.83 (dd, J = 4.4, 1.6 Hz, 2H), 7.48 (d, J = 3.6 Hz, 1H), 7.43 (d, |
| | | J = 3.6 Hz, 1H), 4.95-4.73 (m, 1H), 3.26-3.19 (m, 2H), 2.96- |
| | | 2.86 (m, 2H), 2.00-1.87 (m, 6H), 1.82-1.67 (m, 2H), 0.93- |
| | | 0.91 (m, 2H), 0.67-0.60 (m, 2H). |
| 034 | 405.1 | ¹H NMR (400 MHz, DMSO-d₆) δ 9.57 (s, 1H), 8.65 (dd, J = 4.8, |
| | | 1.6 Hz, 2H), 7.89 (dd, J = 5.6, 2.0 Hz, 2H), 7.83 (dd, J = 4.8 |
| | | Hz, 1.6 Hz, 2H), 7.48 (d, J = 3.6 Hz, 1H), 7.45 (d, J = 3.2 Hz, 1H), |
| | | 4.72 (s, 1H), 3.65-3.60 (m, 1H), 3.24-3.21 (m, 2H), 2.83- |
| | | 2.73 (m, 2H), 1.93-1.81 (m, 3H), 1.65-1.53 (m, 2H), 0.96- |
| | | 0.89 (m, 2H), 0.65-0.59 (m, 2H). |
| 044 | 482.1 | ¹H NMR (400 MHz, DMSO-d₆) δ 9.36 (s, 1H), 8.37 (dd, J = 4.0, |
| | | 16.0 Hz, 2H), 7.26 (d, J = 4.0 Hz, 1H), 6.39 (d, J = 4.0 Hz, 1H), |
| | | 3.90 (dd, J = 4.0, 16.0 Hz, 2H), 3.45-3.42 (m, 4H), 3.04-2.97 (m, |
| | | 4H), 2.10-2.07 (m, 2H), 1.91-1.87 (m, 2H), 1.70-1.59 (m, 2H), |
| | | 1.43-1.40 (m, 2H), 1.07 (s, 3H). |
| 047 | 367.3 | ¹H NMR (400 MHz DMSO-d₆) δ 10.45 (s, 1H), 8.81(d, J = 1.6 |
| | | Hz, 1H), 8.74 (d, J = 8.0 Hz, 1H), 8.42, (d, J = 1.2 Hz, 1H), 8.17 |
| | | (d, J = 2.4 Hz, 1H), 8.15-8.10 (m, 1H), 7.73-7.70 (m, 1H), |
| | | 4.77 (d, J = 1.2 Hz, 1H), 3.70-3.69 (m, 1H), 3.45-3.38 (m, |
| | | 2H), 2.96-2.91 (m, 2H), 1.93-1.88 (m, 2H), 1.70-1.62 (m, |
| | | 2H). |
| 051 | 367.3 | ¹H NMR (400 MHz DMSO-d₆) δ 10.19 (s, 1H), 9.13 (d, J = 1.6 |
| | | Hz, 1H), 8.79 (d, J = 2.0 Hz, 1H), 8.44 (d, J = 1.2 Hz, 1H), |
| | | 8.32-8.29 (m, 1H), 8.09 (d, J = 2.4 Hz, 1H), 7.61-7.58 (m, 1H), |
| | | 4.72 (d, J = 1.2 Hz, 1H), 3.78-3.63 (m, 3H), 3.10-3.03 (m, |
| | | 2H), 1.81-1.77 (m, 2H), 1.50-1.42 (m, 2H). |
| 059 | 522.3 | ¹H NMR (400 MHz, DMSO-d₆) δ 9.36 (s, 1H), 8.43-8.40 (d, J = |
| | | 4.0 Hz, 1H), 8.36 (d, J = 4.0 Hz, 1H), 7.28 (d, J = 4.0 Hz, 1H), |
| | | 6.42 (d, J = 4.0 Hz, 1H), 5.02-4.86 (m, 1H), 3.97-3.84 (m, 2H), |
| | | 3.48-3.42 (m, 2H), 3.23-3.09 (m, 4H), 3.07-2.96 (m, 1H), |
| | | 2.37-2.25 (m, 2H), 1.99 (s, 3H), 1.95-1.86 (m, 2H), 1.86-1.78 |
| | | (m, 2H), 1.76-1.63 (m, 6H). |
| 060 | 480.3 | ¹H NMR (400 MHz, DMSO-d₆) δ 8.68 (d, J = 2.4 Hz, 1H), 7.71- |
| | | 7.64 (m, 1H), 6.51 (d, J = 4.0 Hz, 1H), 6.03 (d, J = 4.0 Hz, 1H), |
| | | 4.15-4.04 (m, 1H), 3.95-3.84 (m, 2H), 3.67-3.51 (m, 4H), |
| | | 3.46-3.40 (m, 2H), 3.19 (s, 1H), 2.93-2.82 (m, 1H), 2.19-2.10 |
| | | (m, 2H), 1.91-1.81 (m, 2H), 1.67-1.49 (m, 8H). |
| 062 | 424.3 | ¹H NMR (400 MHz, DMSO-d₆) δ 10.00 (s, 1H), 8.82 (d, J = 1.2 |
| | | Hz, 1H), 8.13 (d, J = 1.6 Hz, 1H), 7.28 (d, J = 3.2 Hz, 1H), |
| | | 6.43-6.37 (m, 1H), 3.98-3.87 (m, 2H), 3.50-3.42 (m, 3H), 3.39- |
| | | 3.25 (m, 3H), 3.08-2.91 (m, 3H), 1.98-1.86 (m, 2H), 1.76- |
| | | 1.56 (m, 6H). |
| 064 | 501.2 | ¹H NMR (400 MHz, DMSO-d₆) δ 9.97 (s, 1H), 8.67 (d, J = 4.0 |
| | | Hz, 2H), 8.48(d, J = 1.6 Hz, 1H), 8.30 (d, J = 4.0 Hz, 1H), 7.90 |
| | | (d, J = 4.0 Hz, 2H), 7.52-7.48 (m, 2H), 6.04 (s, 1H), 3.82 (d, |
| | | J = 4.0 Hz, 2H), 3.18-3.12 (m, 2H), 1.98-1.91 (m, 2H), 1.75- |
| | | 1.72 (m, 2H). |
| 068 | 477.2 | ¹H NMR (400 MHz, DMSO-d₆) δ 10.45 (s, 1H), 9.23 (d, J = 4.0 |
| | | Hz, 1H), 8.78 (d, J = 3.2 Hz, 1H), 8.56 (d, J = 4.0 Hz, 1H), 8.49 |
| | | (d, J = 4.0 Hz, 1H), 8.39 (d, J = 8.0 Hz, 1H), 4.97-4.91 (m, 1H), |
| | | 3.47-3.41 (m, 2H), 3.18-3.12 (m, 2H), 2.09-2.00 (m, 5H), |
| | | 1.86-1.78 (m, 2H). |
| 069 | 508.2 | ¹H NMR (400 MHz, DMSO-d₆) δ 9.39 (s, 1H), 8.46-8.45 (m, |
| | | 1H), 8.41 (d, J = 2.0 Hz, 1H), 7.28 (d, J = 3.6 Hz, 1H), 6.43 (d, |
| | | J = 4.0 Hz, 1H), 6.06 (s, 1H), 3.97-3.89 (m, 2H), 3.64-3.61 (m, |
| | | 2H), 3.46-3.39 (m, 2H), 3.14-3.09 (m, 2H), 3.02-2.94 (m, |
| | | 1H), 1.96-1.89 (m, 4H), 1.78-1.63 (m, 4H). |
| 070 | 453.21 | ¹H NMR (400 MHz, DMSO-d₆) δ 10.11 (s, 1H), 8.70-8.68 (m, |
| | | 2H), 8.50 (s, 1H), 8.24 (d, J = 2.4 Hz, 1H), 7.91-7.89 (m, 2H), |
| | | 7.51 (s, 2H), 3.54 (t, J = 5.2 Hz, 4H), 2.16-2.06 (m, 4H). |
| 083 | 454.22 | ¹H NMR (400 MHz, DMSO-d₆) δ 9.31 (s, 1H), 8.42 (s, 1H), 8.40- |
| | | 8.39 (m, 1H), 7.27 (d, J = 3.6 Hz, 1H), 6.42 (d, J = 3.2 Hz, 1H), |
| | | 4.50 (s, 1H), 3.94-3 .90 (m, 2H), 3.69-3.66 (m, 2H), 3.48-3.43 |
| | | (m, 2H), 3.30-3.29 (m, 2H), 3.03-3.02 (m, 1H), 2.84-2.79 (m, |
| | | 2H), 1.94-1.90 (m, 2H), 1.79-1.76 (m, 2H), 1.74-1.64 (m, |
| | | 2H), 1.62-1.54 (m, 1H), 1.39-1.29 (m, 2H). |
| 084 | 422.2 | ¹H NMR (400 MHz, DMSO-d₆) δ 13.28 (s, 1H), 9.61 (s, 1H), |
| | | 8.42 (s, 1H), 8.26 (s, 1H), 7.84 (s, 1H), 7.43 (d, J = 2.8 Hz, 1H), |
| | | 6.93 (d, J = 2.8 Hz, 1H), 6.74 (s, 1H), 4.73 (s, 1H), 3.70-3.63 |
| | | (m, 3H), 3.07-3.02 (m, 2H), 1.87-1.84 (m, 2H), 1.56-1.54 (m, |
| | | 2H). |
| 085 | 467.25 | ¹H NMR (400 MHz, DMSO-d₆) δ 9.60 (s, 1H), 8.45 (d, J = 1.2 |
| | | Hz, 1H), 8.26 (d, J = 2.0 Hz, 1H), 7.29 (d, J = 3.6 Hz, 1H), 6.41 |
| | | (d, J = 2.8 Hz, 1H), 3.96-3.87 (m, 2H), 3.65-3.53 (m, 4H), |
| | | 3.52-3.41 (m, 2H), 3.41-3.35 (m, 2H), 3.33-3.31 (m, 2H), |
| | | 3.07-2.97 (m, 1H), 2.02 (s, 3H), 1.96-1.86 (m, 2H), 1.75- |
| | | 1.62 (m, 2H). |
| 086 | 394.31 | ¹H NMR (400 MHz, DMSO-d₆) δ 8.07 (s, 2H), 8.05-8.04 (d, J = |
| | | 2.4 Hz, 1H), 7.92-7.91 (d, J = 2.4 Hz, 1H), 7.32-7.31 (d, J = |
| | | 3.6 Hz, 1H), 6.77-6.76 (d, J = 3.6 Hz, 1H), 3.67 (m, 1H), 3.22- |
| | | 3.17 (m, 2H), 2.80 (m, 2H), 2.00-1.86 (m, 3H), 1.64 (m, 2H), |
| | | 1.02-0.90 (m, 2H), 0.71-0.60 (m, 2H). |
| 087 | 401.21 | ¹H NMR (400 MHz, DMSO-d₆) δ 10.22 (s, 1H), 8.84 (d, J = 2.4 |
| | | Hz, 1H), 8.50-8.43 (m, 1H), 8.23-8.14 (m, 2H), 7.92-7.83 |
| | | (m, 1H), 4.77 (d, J = 3.6 Hz, 1H), 3.81-3.68 (m, 1H), |
| | | 3.46-3.36 (m, 2H), 3.03-2.92 (m, 2H), 2.03-1.91 (m, 2H), |
| | | 1.80-1.67 (m, 2H). |
| 089 | 385.27 | ¹H NMR (400 MHz, DMSO-d₆) δ 10.30 (s, 1H), 8.83-8.74 (m, |
| | | 2H), 8.49-8.42 (m, 1H), 8.28 (dd, J = 8.8, 4.4 Hz, 1H), 8.04 |
| | | (td, J = 8.8, 2.8 Hz, 1H), 4.76 (d, J = 4.0 Hz, 1H), 3.79-3.67 (m, |
| | | 1H), 3.49-3.39 (m, 2H), 3.03-2.93 (m, 2H), 1.98-1.87 (m, |
| | | 2H), 1.73-1.59 (m, 2H). |
| 090 | 397.27 | ¹H NMR (400 MHz, DMSO-d₆) δ 10.22 (s, 1H), 8.84 (d, J = 2.4 |
| | | Hz, 1H), 8.50-8.43 (m, 1H), 8.23-8.14 (m, 2H), 7.92-7.83 |
| | | (m, 1H), 4.77 (d, J = 3.6 Hz, 1H), 3.81-3.68 (m, 1H), |
| | | 3.46-3.36 (m, 2H), 3.03-2.92 (m, 2H), 2.03-1.91 (m, 2H), |
| | | 1.80-1.67 (m, 2H). |
| 091 | 401.16 | ¹H NMR (400 MHz, DMSO-d₆) δ 10.40 (s, 1H), 8.81-8.70 (m, |
| | | 2H), 8.49-8.43 (m, 1H), 8.20 (d, J = 2.0 Hz, 1H), 7.91 (dd, J = |
| | | 5.2, 2.0 Hz, 1H), 4.77 (d, J = 3.6 Hz, 1H), 3.81-3.65 (m, 1H), |
| | | 3.52-3.38 (m, 2H), 3.02-2.91 (m, 2H), 2.01-1.85 (m, 2H), |
| | | 1.75-1.57 (m, 2H). |
| 092 | 435.2 | ¹H NMR (400 MHz, DMSO-d₆) δ 13.31 (s, 1H), 9.48 (s, 1H), |
| | | 8.61 (s, 1H), 8.10 (s, 2H), 7.47 (d, J = 1.6 Hz, 2H), 7.36 (d, J = |
| | | 3.6 Hz, 1H), 6.81 (d, J = 3.6 Hz, 1H), 4.42 (s, 1H), 3.09-3.03 |
| | | (m, 2H), 2.85-2.80 (m, 2H), 1.83-1.68 (m, 4H), 1.23 (s, 3H). |
| 093 | 401.1 | ¹H NMR (400 MHz, DMSO-d₆) δ 10.32 (s, 1H), 8.83 (d, J = 2.0 |
| | | Hz, 1H), 8.75 (d, J = 2.0 Hz, 1H), 8.44 (dd, J = 2.8, 1.2 Hz, 1H), |
| | | 8.27-8.25 (m, 1H), 8.21-8.19 (m, 1H), 4.74 (d, J = 4.0 Hz, 1H), |
| | | 3.75-3.67 (m, 1H), 3.46-3.41 (m, 2H), 3.02-2.91 (m, 2H), |
| | | 1.97-1.86 (m, 2H), 1.71-1.59 (m, 2H). |
| 098 | 400 | ¹H NMR (400 MHz, DMSO-d₆) δ 13.57 (s, 1H), 9.89 (s, 1H), |
| | | 8.44 (d, J = 1.2 Hz, 1H), 8.16 (d, J = 2.1 Hz, 1H), 7.45 (d, J = 3.6 |
| | | Hz, 1H), 7.37 (d, J = 3.6 Hz, 1H), 4.70 (s, 1H), 3.82-3.54 (m, |
| | | 3H), 3.05 (t, J = 11.2 Hz, 2H), 1.85-1.71 (m, 2H), 1.55-1.38 |
| | | (m, 2H). |
| 101 | 440.3 | ¹HNMR (400 MHz DMSO-d₆) δ 9.47 (s, 1H), 8.44 (s, 1H), 8.39 |
| | | (d, J = 3.2 Hz, 1H), 8.07 (s, 1H), 8.03 (d, J = 2.4 Hz, 1H), 4.71 |
| | | (d, J = 4.8 Hz, 1H), 4.52-4.46 (m, 1H), 3.99-3.95 (m, 2H), |
| | | 3.71-3.66 (m, 3H), 3.51-3.44 (m, 2H), 3.06-3.01 (m, 2H), |
| | | 2.00-1.93 (m, 4H), 1.82-1.78 (m, 2H), 1.51-1.48 (m, 2H). |
| 102 | 452.2 | ¹HNMR (400 MHz, DMSO-d₆) δ 10.15 (s, 1H), 8.41 (s, 1H), |
| | | 8.07 (s, 1H), 7.40 (d, J = 3.6 Hz, 1H), 7.30 (d, J = 4.0 Hz, 1H), 6.85 |
| | | (s, 1H), 4.68 (d, J = 4.0 Hz, 1H), 4.48 (t, J = 6.4 Hz, 2H), 3.79-3.65 |
| | | (m, 3H), 3.11-3.05 (m, 2H), 2.86 (t, J = 5.6 Hz, 2H), 1.81-1.77 |
| | | (m, 2H), 1.50-1.41 (m, 2H). |
| 103 | 440.3 | ¹HNMR (400 MHz, DMSO_{-d6}) δ 9.40 (s, 1H), 8.70 (s, 1H), 8.41 |
| | | (s, 1H), 8.06 (d, J = 2.0 Hz, 1H), 6.84 (d, J = 2.0 Hz, 1H), 4.82 |
| | | (d, J = 4.0 Hz, 1H), 4.62-4.49 (m, 1H), 4.04-3.94 (m, 2H), |
| | | 3.78-3.68 (m, 1H), 3.54-3.44 (m, 2H), 3.43-3.36 (m, 2H), |
| | | 3.01-2.88 (m, 2H), 2.12-1.88 (m, 6H), 1.73-1.59 (m, 2H). |

### Example 2—Preparation of N-(2-(4-hydroxypiperidin-1-yl)-5-(trifluoromethyl)pyridin-3-yl)-5-(pyridine-4-yl) furan-2-carboxamide (Compound 004)

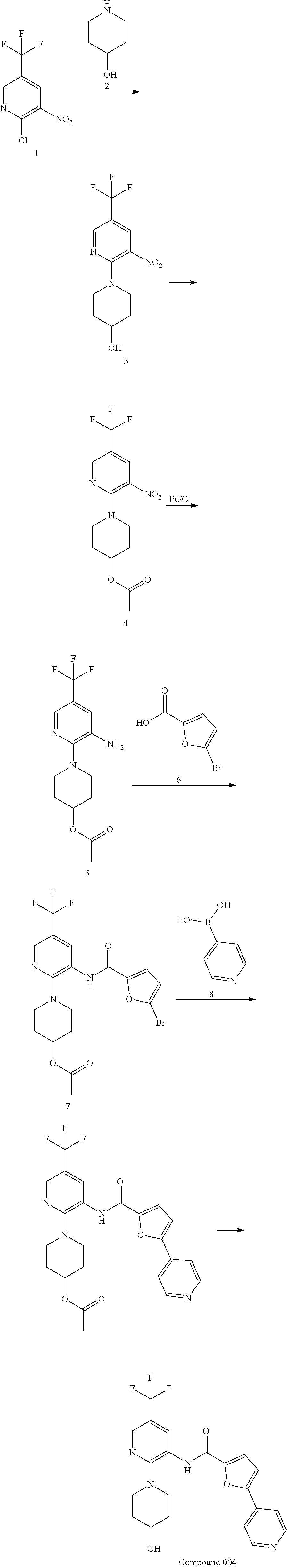

Step-1. To a solution of 2-chloro-3-nitro-5-(trifluoromethyl)pyridine (1 g, 4.4 mmol) and piperidin-4-ol (892 mg, 8.8 mmol) in DMF (10 mL) was added K₂CO₃ (1.22 g, 8.8 mmol). The reaction mixture was stirred overnight at 80° C. LCMS showed the reaction was completed. The reaction mixture was poured on to water (10 ml) and extracted with EA (3×6 ml), brine (6 ml), which was dried over anhydrous Na₂SO₄, filtered, concentrated in vacuum to give the desired product 1-(3-nitro-5-(trifluoromethyl)pyridin-2-yl) piperidin-4-ol. LCMS: 292 [M+1]; Retention time: 1.45 min.

Step-2. To a solution of 1-(3-nitro-5-(trifluoromethyl)pyridin-2-yl) piperidin-4-ol (1 g, 3.4 mmol) and TEA (1.45 mL, 9 mmol) in DMF (20 mL) cooled to 0° C. was added Acetyl chloride (0.4 mL, 3.6 mmol). The reaction mixture was stirred overnight at room temperature. LCMS showed the reaction was completed. The reaction mixture was poured on to water (10 ml) and extracted with EA (3×6 ml), brine (6 ml), which was dried over anhydrous Na₂SO₄, filtered, and evaporated to give crude product. The crude product was purified by column chromatography on silica gel eluted to give desired compound 1-(3-nitro-5-(trifluoromethyl)pyridin-2-yl) piperidin-4-yl acetate. LCMS: 334 [M+1]; Retention time: 1.64 min.

Step-3. To a solution of 1-(3-nitro-5-(trifluoromethyl)pyridin-2-yl) piperidin-4-yl acetate (640 mg, 1.92 mmol) and Pd/C (64 mg, 0.6 mmol) in MeOH (30 mL) was stirred under 1 atm of H₂ at room temperature for 4 h. LCMS showed the reaction was completed. The reaction mixture was filtered with diatomaceous earth, and the filtrate was concentrated to give the desired product 1-(3-amino-5-(trifluoromethyl)pyridin-2-yl) piperidin-4-yl acetate. LCMS: 304 [M+1]; Retention time: 1.98 min.

Step-4. To a solution of 2-(4-methylpiperazin-1-yl)-5-(trifluoromethyl)pyridin-3-amine (1 g, 3.85 mmol), 5-bromofuran-2-carboxylic acid (735 mg, 3.85 mmol) and Pyridine (0.93 mL, 11.54 mmol) in DCM (15 mL) cooled to 0° C. was added POCl₃ (0.7 mL, 7.7 mmol). The reaction mixture was stirred overnight at room temperature. LCMS showed the reaction was completed. The reaction mixture was poured on to water (5 ml) and extracted with EA (3× 3 ml), brine (6 ml), which was dried over anhydrous Na₂SO₄, filtered, and evaporated to give crude product. The crude product was purified by prep-HPLC (TFA) to give the desired product 5-bromo-N-(2-(4-methylpiperazin-1-yl)-5-(trifluoromethyl)pyridin-3-yl) furan-2-carboxamide. LCMS: 435 [M+1]; Retention time: 1.22 min.

Step-5. To solution of 5-bromo-N-(2-(4-methylpiperazin-1-yl)-5-(trifluoromethyl)pyridin-3-yl) furan-2-carboxamide (60 mg, 0.13 mmol), pyridin-4-ylboronic acid (31 mg, 0.26 mmol), K₂CO₃ (35 mg, 0.26 mmol), and DPPF (7 mg, 0.01 mmol) in 1,4-Dioxane (4 mL) and H₂O (1 mL). The reaction mixture was stirred at 90° C. for 7 h. LCMS showed the reaction was completed. The reaction mixture was poured on to water (8 ml) and extracted with EA (3×6 ml), brine (6 ml), which was dried over anhydrous Na₂SO₄, filtered, and evaporated to give crude product. The crude product was purified by prep-HPLC (TFA) to give desired compound 1-(3-(5-(pyridin-4-yl) furan-2-carboxamido)-5-(trifluoromethyl)pyridin-2-yl) piperidin-4-yl acetate. LCMS: 475 [M+1]; Retention time: 1.36 min.

Step-6. To a solution of SM1 (56 mg, 0.12 mmol) in MeOH (1 mL) and THF (1 mL) was added K₂CO₃ (18 mg, 0.13 mmol). The reaction mixture was stirred at room temperature for 4 h. LCMS showed the reaction was completed. The mixture was evaporated to afford the crude product. The crude product was purified by prep-HPLC (TFA) to give desired compound. LCMS: 433 [M+1]; Retention time: 1.26 min

¹H NMR (400 MHz, CDCl₃) δ 9.03-8.98 (m, 1H), 8.96-8.88 (m, 1H), 8.69-8.63 (m, 2H), 8.32-8.27 (m, 1H), 7.60-7.55 (m, 2H), 7.38-7.31 (m, 1H), 7.01-6.94 (m, 1H), 5.34-5.23 (m, 1H), 4.01-3.89 (m, 1H), 3.38-3.21 (m, 2H), 3.02-2.95 (m, 2H), 2.13-2.08 (m, 2H), 1.86-1.77 (m, 2H).

The following compounds were prepared in a similar manner to the procedure described above.

| | LCMS | |
|---|---|---|
| Compound | (ESI): | |
| No. | [M + H]⁺ | ¹H NMR |
| 003 | 432.0 | ¹H NMR (400 MHz, CDCl₃) δ 9.08-8.95 (m, 2H), 8.77 (d, J = |
| | | 5.7 Hz, 2H), 8.45-8.38 (m, 1H), 7.70 (d, J = 6.0 Hz, 2H), 7.44 |
| | | (s, 1H), 7.10 (d, J = 3.6 Hz, 1H), 3.29 (s, 4H), 2.89-2.70 (m, 4H), |
| | | 2.46 (s, 3H). |
| 006 | 432.0 | ¹H NMR (400 MHz, CDCl₃) δ 9.11 (s, 1H), 8.76 (d, J = 2.3 Hz, |
| | | 1H), 8.66 (d, J = 5.9 Hz, 2H), 8.03 (d, J = 2.4 Hz, 1H), 7.62 (d, |
| | | J = 6.1 Hz, 2H), 7.33 (d, J = 3.7 Hz, 1H), 6.99 (s, 1H), 3.07 (t, J = |
| | | 4.8 Hz, 4H), 2.65 (s, 4H). |
| 017 | 462.0 | ¹H NMR (400 MHz, DMSO-d₆) 10.03-9.91 (m, 1H), 8.53-8.37 |
| | | (m, 1H), 8.21-8.07 (m, 1H), 7.59-7.50 (m, 1H), 6.31 (s, 2H), |
| | | 3.77-3.60 (m, 3H), 3.09-2.99 (m, 2H), 1.85-1.76 (m, 2H), |
| | | 1.53-1.41 (m, 2H) |
| 018 | 420.0 | ¹H NMR (400 MHz, DMSO-d₆) 9.50 (s, 1H), 8.23-7.86 (m, 4H), |
| | | 7.42 (d, J = 3.6 Hz, 1H), 7.29 (d, J = 3.6 Hz, 1H), 6.98 (d, J = 5.1 |
| | | Hz, 1H), 6.89 (s, 1H), 6.16 (s, 2H), 3.74-3.61 (m, 1H), 3.17 (d, |
| | | J = 12.4 Hz, 2H), 2.84 (t, J = 10.8 Hz, 2H), 2.03-1.87 (m, 3H), |
| | | 1.69 (dd, J = 19.9, 9.3 Hz, 2H), 1.04-0.92 (m, 2H), 0.71-0.58 |
| | | (m, 2H) |
| 020 | 435.0 | ¹H NMR (400 MHz, DMSO-d₆) 9.45 (s, 1H), 8.17 (d, J = 2.2 Hz, |
| | | 1H), 7.92 (t, J = 11.4 Hz, 1H), 7.37 (d, J = 3.6 Hz, 1H), 7.03 (d, |
| | | J = 3.6 Hz, 1H), 6.12 (s, 2H), 5.61 (s, 4H), 3.68 (dd, J = 9.4, 5.0 Hz, |
| | | 1H), 3.10 (s, 2H), 2.83 (t, J = 10.9 Hz, 3H), 2.00-1.87 (m, 3H), |
| | | 1.75-1.64 (m, 2H), 1.23 (s, 2H), 1.02-0.91 (m, 2H), 0.71-0.62 |
| | | (m, 2H) |
| 021 | 449.0 | ¹H NMR (400 MHz, DMSO-d₆) 11.71-11.58 (m, 1H), 10.21- |
| | | 9.99 (m, 1H), 10.15-10.04 (m, 1H), 8.48-8.39 (m, 1H), 8.11- |
| | | 8.02 (m, 1H), 7.42 (s, 3H), 7.07-6.94 (m, 1H), 6.72-6.61 (m, |
| | | 1H), 4.71-4.65 (m, 1H), 3.78-3.62 (m, 3H), 3.12-3.02 (m, |
| | | 2H), 2.02-1.95 (m, 2H), 1.82-1.75 (m, 1H). |
| 022 | 421.0 | ¹H NMR (400 MHz, DMSO-d₆) 9.70 (s, 1H), 7.93 (dd, J = 22.0, |
| | | 2.1 Hz, 2H), 7.57-7.32 (m, 3H), 6.96 (s, 1H), 6.70 (d, J = 6.3 Hz, |
| | | 1H), 3.69-3.59 (m, 1H), 3.23 (d, J = 12.5 Hz, 2H), 2.82 (t, J = |
| | | 10.1 Hz, 2H), 1.96-1.80 (m, 3H), 1.61 (dd, J = 18.8, 9.2 Hz, 2H), |
| | | 1.23 (s, 1H), 1.00-0.89 (m, 2H), 0.74-0.57 (m, 2H). |
| 023 | 423.0 | ¹H NMR (400 MHz, DMSO-d₆) 9.69 (s, 1H), 8.36 (d, J = 5.2 Hz, |
| | | 1H), 7.97 (d, J = 2.0 Hz, 1H), 7.88-7.74 (m, 2H), 7.71 (s, 1H), |
| | | 7.61 (d, J = 3.6 Hz, 1H), 7.46 (d, J = 3.6 Hz, 1H), 4.73 (s, 1H), |
| | | 3.73-3.59 (m, 1H), 3.28 (d, J = 12.5 Hz, 3H), 2.89-2.66 (m, |
| | | 2H), 1.92 (ddd, J = 25.1, 15.1, 7.6 Hz, 3H), 1.59 (dd, J = 18.7, 9.1 |
| | | Hz, 2H), 1.03-0.88 (m, 2H), 0.74-0.53 (m, 2H). |
| 049 | 476.1 | ¹H NMR (400 MHz, DMSO-d₆) δ 10.27 (s, 1H), 9.86-9.80 (m, |
| | | 1H), 9.35 (dd, J = 4.0, 1.2 Hz, 1H), 8.49-8.41 (m, 1H), 8.19- |
| | | 8.08 (m, 2H), 7.70 (d, J = 4.0 Hz, 1H), 7.53 (d, J = 4.0 Hz, 1H), |
| | | 4.94-4.78 (m, 1H), 3.79-3.66 (m, 2H), 3.30-3.17 (m, 2H), |
| | | 1.99 (s, 3H), 1.97-1.88 (m, 2H), 1.73-1.61 (m, 2H). |
| 053 | 422.3 | ¹H NMR (400 MHz, DMSO-d₆) δ 13.26 (s, 1H), 9.52 (s, 1H), 8.43- |
| | | 8.01 (m, 4H), 7.38 (d, J = 4.0 Hz, 1H), 6.78 (d, J = 4.0 Hz, 1H), |
| | | 4.78 (d, J = 4.0 Hz, 1H), 3.71-3.61 (m, 3H), 3.06-3.00 (m, 2H), |
| | | 1.88-1.83 (m, 2H), 1.59-1.52 (m, 2H). |
| 056 | 433.2 | ¹H NMR (400 MHz, DMSO-d₆) δ 10.05 (s, 1H), 9.39 (s, 2H), 9.20 |
| | | (s, 1H), 8.48-8.41 (m, 1H), 8.10 (d, J = 2.4 Hz, 1H), 7.48 (s, 2H), |
| | | 4.70 (d, J = 4.0 Hz, 1H), 3.80-3.71 (m, 2H), 3.70-3.64 (m, 1H), |
| | | 3.13-3.05 (m, 2H), 1.85-1.76 (m, 2H), 1.53-1.44 (m, 2H). |
| 061 | 439.3 | ¹H NMR (400 MHz, DMSO-d₆) δ 9.38 (s, 1H), 8.41 (s, 1H), 8.32 |
| | | (s, 1H), 7.26 (d, J = 3.2 Hz, 1H), 6.37 (d, J = 3.2 Hz, 1H), 4.76 (s, |
| | | 1H), 3.72-3.66 (m, 1H), 3.59-3.54 (m, 2H), 3.02-2.94 (m, 4H), |
| | | 2.85-2.77 (m, 1H), 2.62-2.54 (m, 2H), 1.91-1.84 (m, 4H), |
| | | 1.60-1.48 (m, 4H). |
| 063 | 434.23 | ¹H NMR (400 MHz, DMSO-d₆) δ 10.05 (s, 1H), 9.39 (s, 2H), 9.20 |
| | | (s, 1H), 8.45 (d, J = 1.2 Hz, 1H), 8.10 (d, J = 2.4 Hz, 1H), 7.48 (s, |
| | | 2H), 4.70 (d, J = 4.0 Hz, 1H), 3.81-3.72 (m, 2H), 3.71-3.63 (m, |
| | | 1H), 3.14-3.04 (m, 2H), 1.87-1.74 (m, 2H), 1.54-1.43 (m, 2H). |
| 099 | 448 | ¹H NMR (400 MHz, DMSO-d₆) δ 12.42-11.78 (m, 1H), 9.89- |
| | | 9.76 (m, 1H), 8.47-8.36 (m, 1H), 8.17-8.02 (m, 2H), 8.02- |
| | | 7.85 (m, 1H), 7.39-7.28 (m, 1H), 6.97-6.88 (m, 1H), 6.58- |
| | | 6.41 (m, 1H), 4.77-4.58 (m, 1H), 3.77-3.57 (m, 3H), 3.14- |
| | | 2.97 (m, 2H), 2.04-1.91 (m, 2H), 1.87-1.70 (m, 2H), 1.51- |
| | | 1.36 (m, 2H). |

### Example 3: Preparation of N-[2-(4-hydroxy-4-methyl-1-piperidyl)-5-(trifluoromethyl)-3-pyridyl]-5-tetrahydropyran-4-yl-furan-2-carboxamide (Compound 027)

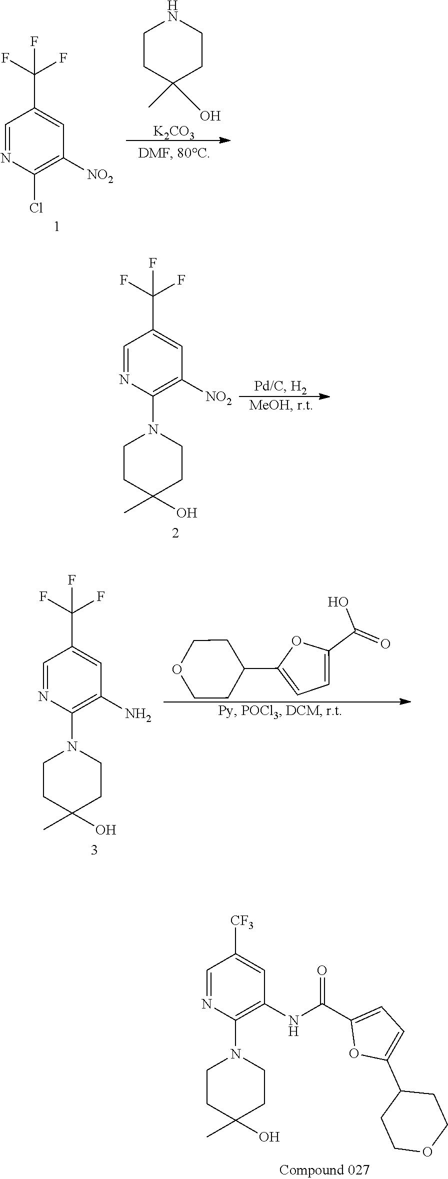

Step-1. To a solution of 4-methylpiperidin-4-ol (305 mg, 2.65 mmol) and 2-chloro-3-nitro-5-(trifluoromethyl)pyridine (500 mg, 2.21 mmol) in N,N-dimethylformamide (5 mL) was added potassium carbonate (610 mg, 4.41 mmol). The mixture was heated at 80° C. for 2 hours. TLC analysis showed target product formed. After being cooled, the reaction mixture was diluted with ethyl acetate (50 mL) and washed with water (30 mL×3) and brine (30 mL×3). The organic layer was dried over anhydrous sodium sulfate, filtered, and concentrated under reduced pressure. The crude product was purified by silica column chromatography (petroleum ether:ethyl acetate=7:1) to give 4-methyl-1-[3-nitro-5-(trifluoromethyl)-2-pyridyl]piperidin-4-ol.

Step-2. To a solution of 4-methyl-1-[3-nitro-5-(trifluoromethyl)-2-pyridyl]piperidin-4-ol (1 g, 3.28 mmol) in methanol (30 mL) was added Pd/C (1 g, wet) under N₂. The mixture was degassed with H₂ for three times and mixture was stirred at 25° C. under H₂ (15 psi) for 2 hours. LCMS analysis showed target product formed. The reaction mixture was filtered and concentrated under reduced pressure to give 1-[3-amino-5-(trifluoromethyl)-2-pyridyl]-4-methyl-piperidin-4-ol. LCMS (ESI) m/z: [M+H]⁺ 276.0; Purity=95% (254 nm); Retention time=1.24 min.

Step-3. To a solution of 5-tetrahydropyran-4-ylfuran-2-carboxylic acid (71 mg, 0.36 mmol) and 1-[3-amino-5-(trifluoromethyl)-2-pyridyl]-4-methyl-piperidin-4-ol (100 mg, 0.36 mmol) in dichloromethane (5 mL) was added pyridine (575 mg, 7.27 mmol) under nitrogen. The mixture was stirred at 0° C. for 10 minutes. Then phosphoryl trichloride (1.11 g, 7.27 mmol) was added dropwise slowly under nitrogen at 0° C. The mixture was stirred at 25° C. for 0.5 hours. LCMS analysis showed target product formed. The reaction mixture was quenched by water and diluted with ethyl acetate (50 mL) and washed with water (30 mL×3) and brine (30 mL×3). The organic layer was dried over anhydrous sodium sulfate, filtered, and concentrated under reduced pressure. The crude product was purified by silica column chromatography (petroleum ether:ethyl acetate=3:1) to give N-[2-(4-hydroxy-4-methyl-1-piperidyl)-5-(trifluoromethyl)-3-pyridyl]-5-tetrahydropyran-4-yl-furan-2-carboxamide.

¹HNMR (400 MHz, DMSO-d6) δ 9.33 (s, 1H), 8.41 (d, J=4.0 Hz, 1H), 8.35 (d, J=4.0 Hz, 1H), 7.27 (d, J=4.0 Hz, 1H), 6.42 (d, J=4.0 Hz, 2H), 4.38 (s, 1H), 3.93-3.89 (m, 2H), 3.47-3.41 (m, 3H), 3.26-3.19 (m, 2H), 3.04-2.98 (m, 1H), 1.92 (d, J=4.0 Hz, 2H), 1.89-1.57 (m, 6H), 1.17 (s, 3H).

LCMS (ESI) m/z: [M+H]⁺ 454.1; Purity=94% (254 nm); Retention time=1.51 min.

### Example 4: Preparation of methyl 4-methyl-1-[3-[(5-tetrahydropyran-4-ylfuran-2-carbonyl)amino]-5-(trifluoromethyl)-2-pyridyl]piperidine-4-carboxylate (Compound 032)

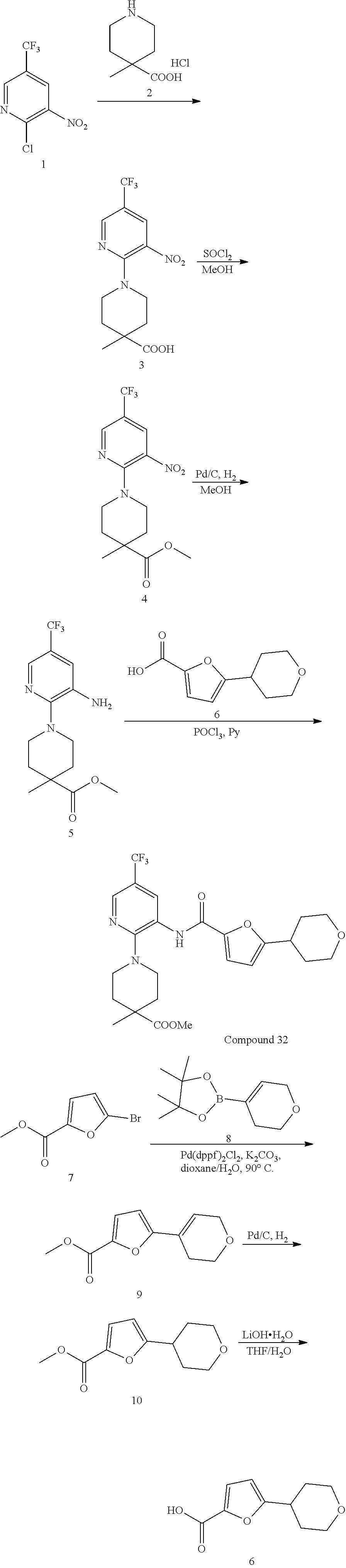

Step-1. To a solution of 2-chloro-3-nitro-5-(trifluoromethyl)pyridine (4.2 g, 18.54 mmol) in DMF (30 mL) was added 4-methylpiperidine-4-carboxylic acid (2.92 g, 20.39 mmol, HCl salt) and K₂CO₃ (7.68 g, 55.62 mmol), the mixture solution was stirred at 80° C. for 1.5 hr, the reaction was monitored by LCMS, it showed the starting material was consumed up and product formed. Water (20 mL) was added and adjusted to pH=5-6 by 2 N HCl, extracted with EA (50 mL*3), the combined organic layer was dried over anhydrous Na₂SO₄, filtered and concentrated to give the crude product (4-methyl-1-[3-nitro-5-(trifluoromethyl)-2-pyridyl]piperidine-4-carboxylic acid without further purification.

Step-2. To a solution of 4-methyl-1-[3-nitro-5-(trifluoromethyl)-2-pyridyl]piperidine-4-carboxylic acid (500 mg, 1.50 mmol) in MeOH (10 mL) was added Thionyl chloride (2 mL) slowly, and the reaction solution was stirred at 80° C. for 2 hr. TLC showed the starting material was consumed up and product formed, solvent and thionyl chloride was removed under reduced pressure to give a residue. The residue was purified by Flash chromatography column with eluting PE:EA=9:1 to give methyl 4-methyl-1-[3-nitro-5-(trifluoromethyl)-2-pyridyl]piperidine-4-carboxylate.

Step-3. To a solution of methyl 4-methyl-1-[3-nitro-5-(trifluoromethyl)-2-pyridyl]piperidine-4-carboxylate (489 mg, 1.41 mmol) in MeOH (10 mL) was added Pd/C (wet) (50 mg), and the mixture solution was stirred at 25° C. under hydrogen atmosphere (15 psi) for 4 hr, TLC showed that the starting material was consumed up and the product formed, the reaction solution was filtered and the filtrate was concentrated to give methyl 1-[3-amino-5-(trifluoromethyl)-2-pyridyl]-4-methyl-piperidine-4-carboxylate.

Step-4. To a solution of methyl 5-bromofuran-2-carboxylate (5.85 g, 28.54 mmol) in Dioxane (50 mL) and H₂O (10 mL) was added 2-(3,6-dihydro-2H-pyran-4-yl)-4,4,5,5-tetramethyl-1,3,2-dioxaborolane (7.19 g, 34.24 mmol), K₂CO₃ (11.81 g, 85.61 mmol) and Pd(dppf)Cl₂ (2.07 g, 2.85 mmol), the mixture solution was stirred at 90° C. under nitrogen atmosphere for 4 hr, TLC showed starting material was consumed up fully, product formed, then dioxane was removed to give a residue, water (50 mL) was added, and extracted with EA (50 mL*3), the combine organic layer was dried over anhydrous Na₂SO₄, filtered to give the residue. The residue was purified by Flash chromatography column with eluting PE:EA (5:1) to give methyl 5-(3,6-dihydro-2H-pyran-4-yl) furan-2-carboxylate.

Step-5. To a solution of methyl 5-(3,6-dihydro-2H-pyran-4-yl) furan-2-carboxylate (4.04 g, 19.40 mmol) in MeOH (50 mL) was added Pd/C (wet) (400 mg), and the mixture solution was stirred at 25° C. under hydrogen atmosphere (15 psi) for 2 hr, the reaction was monitored by LCMS and TLC, it showed starting material was consumed up, the reaction solution was filtered, the filtrate was concentrated to give methyl 5-tetrahydropyran-4-ylfuran-2-carboxylate as a colorless oil.

Step 6. To a solution of methyl 5-tetrahydropyran-4-ylfuran-2-carboxylate (5.2 g, 24.74 mmol) in THF (9.17 mL) and H₂O (1 mL) was added Lithium hydroxide monohydrate (2.08 g, 49.47 mmol, 1.37 mL), and the mixture solution was stirred at 25° C. for 16 hr, the reaction was monitored by TLC, it showed starting material was consumed up and the product formed, THF was removed under reduced pressure to give a residue, water was added, acidified by 2N HCl to pH=5, and extracted with EA (50 mL*3), the combined organic layer was dried over anhydrous Na₂SO₄, filtered and concentrated to give 5-tetrahydropyran-4-ylfuran-2-carboxylic acid.

Step-7. To a solution of methyl 1-[3-amino-5-(trifluoromethyl)-2-pyridyl]-4-methyl-piperidine-4-carboxylate (50 mg, 0.16 mmol) in DCM (10 mL) was added 5-tetrahydropyran-4-ylfuran-2-carboxylic acid (31 mg, 0.16 mmol) and pyridine (1.2 mL) at 0° C., and the reaction solution was stirred at 0° C. for 5 min, then POCl₃ (1 mL) was added, the resulting solution was stirred at 0° C. for 10 min, then the reaction solution was stirred at room temperature for 1.2 h. TLC showed the starting material was consumed up and the product formed, the solution was poured to ice-water (10 mL) and extracted with EA (20 mL*2), the combined organic layer was dried over anhydrous Na₂SO₄, filtered and concentrated to give a residue. The residue was purified by prep-TLC (DCM:MeOH=95:5) to give methyl 4-methyl-1-[3-[(5-tetrahydropyran-4-ylfuran-2-carbonyl)amino]-5-(trifluoromethyl)-2-pyridyl]piperidine-4-carboxylate.

¹HNMR (400 MHz, DMSO-d6) δ 9.34 (s, 1H), 8.39-8.38 (m, 1H), 8.34 (d, J=2.4 Hz, 1H), 7.25 (d, J=3.6 Hz, 1H), 6.4 (dd, J=3.6, 0.8 Hz, 1H), 3.91-3.86 (m, 2H), 3.62 (s, 3H), 3.45-3.36 (m, 4H), 3.02-2.95 (m, 3H), 2.11-2.07 (m, 2H), 1.90-1.86 (m, 2H), 1.72-1.54 (m, 4H), 1.17 (s, 3H).

LCMS (ESI) m/z: [M+H]⁺ 496.1; Purity=95% (254 nm); Retention time=1.77 min.

The following compounds were prepared in a similar manner to the procedure described above.

| | LCMS | |
|---|---|---|
| Compound | (ESI): | |
| No. | [M + H]⁺ | ¹H NMR |
| 104 | 422.2 | ¹H NMR (400 MHz, CDCl₃) δ 12.48 (s, 1H), 9.53 (s, 1H), 8.46- |
| | | 8.39 (m, 1H), 8.30-8.23 (m, 1H), 7.78 (s, 1H), 7.62 (s, 1H), 7.40 |
| | | (d, J = 4.0 Hz, 1H), 6.74 (d, J = 4.0 Hz, 1H), 4.75 (d, J = 4.0 Hz, |
| | | 1H), 3.74-3.58 (m, 3H), 3.08-2.99 (m, 2H), 1.90-1.82 (m, |
| | | 2H), 1.61-1.50 (m, 2H). |
| 105 | 423.2 | ¹HNMR (400 MHz, DMSO-d₆) δ 9.85 (s, 1H), 8.55-8.39 (m, |
| | | 2H), 8.18 (d, J = 2.0 Hz, 1H), 7.53 (d, J = 4.0 Hz, 1H), 7.08 |
| | | (d, J = 4.0 Hz, 1H), 4.73 (s, 1H), 3.74-3.61 (m, 3H), 3.11-2.99 (m, |
| | | 2H), 1.91-1.78 (m, 2H), 1.61-1.47 (m, 2H). |

### Example 5: Preparation of 1-(5-cyclopropyl-3-(5-(pyridin-4-yl) furan-2-carboxamido) pyridin-2-yl) piperidin-4-yl acetate (Compound 033)

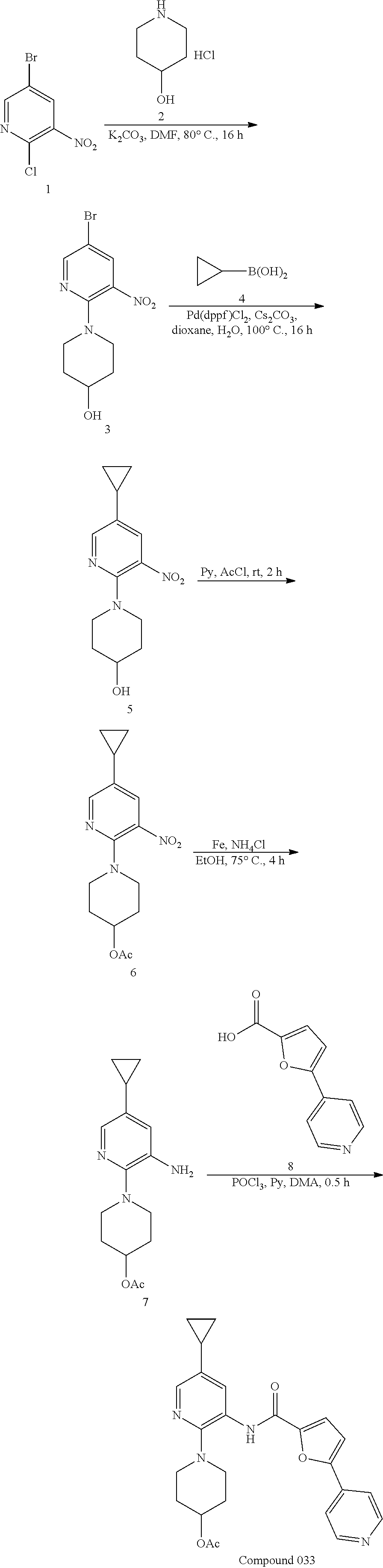

Step-1. To a solution of 5-bromo-2-chloro-3-nitropyridine (2.36 g, 20 mmol) in DMF (30 mL) was added 4-piperidinol hydrochloride (4.11 g, 30 mmol) and K₂CO₃ (8.28 g, 60 mmol), then the reaction solution was stirred at 80° C. for 16 h. The reaction was monitored by TLC, it showed the desire product formed. The solvent was extracted with ethyl acetate (100 mL) and water (200 mL). The combined organic layer was dried with anhydrous sodium sulfate, filtered and concentrated to give a residue and purified by flash chromatography column with eluting PE:EA=3:1 to give 1-(5-bromo-3-nitropyridin-2-yl) piperidin-4-ol.

Step-2. To a solution of 1-(5-bromo-3-nitropyridin-2-yl), piperidin-4-ol (2.5 g, 8.3 mmol) and cyclopropylboronic acid (0.86 g, 10 mmol) in dioxane (50 mL) and water (50 mL) was added cesium carbonate (8 g 24.9 mmol) and [1,1′-Bis(diphenylphosphino) ferrocene]dichloropalladium (II) (0.59 g, 0.83 mmol). The mixture solution was stirred at 100° C. for 16 h under nitrogen atmosphere. The reaction was monitored by TLC, it showed the starting material was consumed up and the desired product formed, extracted with ethyl acetate (100 mL) and water (150 mL). The combined organic layer was dried with anhydrous sodium sulfate, filtered and concentrated to give a residue and purified by flash chromatography column with eluting PE:EtOAc=4:1 to give 1-(5-cyclopropyl-3-nitropyridin-2-yl) piperidin-4-ol.

Step-3. To a solution of 1-(5-cyclopropyl-3-nitropyridin-2-yl) piperidin-4-ol (1.8 g, 7.8 mmol) in pyridine (30 mL) was added acetyl chloride (1.2 g, 15.6 mmol). The mixture solution was stirred at rt for 2 h under nitrogen atmosphere. The reaction was monitored by TLC, it showed the starting material was consumed up and the desire product formed, extracted with ethyl acetate (60 mL) and water (80 mL). The combined organic layer was dried with anhydrous sodium sulfate, filtered and concentrated to give a residue and purified by flash chromatography column with eluting PE:EtOAc=5:1 to give 1-(5-cyclopropyl-3-nitropyridin-2-yl) piperidin-4-yl acetate.

Step-4. To a solution of 1-(5-cyclopropyl-3-nitropyridin-2-yl) piperidin-4-yl acetate (1.3 g, 4.3 mmol) in EtOH (50 mL) and water (10 mL) was added Fe (2.4 g, 43 mmol) and ammonium chloride (1.2 g, 21.5 mmol). The mixture solution was stirred at 75° C. for 4 h under nitrogen atmosphere. The reaction was monitored by TLC, it showed the starting material was consumed up and the desire product formed, the iron was filtered out to give a residue, which was extracted with ethyl acetate (50 mL) and water (70 mL). The combined organic layer was dried with anhydrous sodium sulfate, filtered, and concentrated to give a residue and purified by flash chromatography column with eluting PE:EtOAc=1:5 to give 1-(3-amino-5-cyclopropylpyridin-2-yl) piperidin-4-yl acetate.

Step-5. To a solution of 1-(3-amino-5-cyclopropylpyridin-2-yl) piperidin-4-yl acetate (200 mg, 0.73 mmol) and 5-(pyridin-4-yl) furan-2-carboxylic acid (138 mg, 0.73 mmol) in DMA (20 mL) and pyridine (20 mL) was added POCl₃ (335 mg, 2.19 mmol) at 0° C. under nitrogen atmosphere, and then the mixture was stirred at 25° C. for 0.5 h, TLC showed the starting material was consumed up and the desire product formed, water (80 mL) was added to quench the reaction, and extracted with EA (40 mL), The combine organic layer was washed with brine (30 mL), dried over anhydrous Na₂SO₄, filtered and concentrated to give a residue. The residue was purified by flash chromatography column with eluting PE:EtOAc=1:5 to give 1-(5-cyclopropyl-3-(5-(pyridin-4-yl) furan-2-carboxamido) pyridin-2-yl) piperidin-4-yl acetate.

¹HNMR (400 MHz, DMSO-d₆) δ 9.57 (s, 1H), 8.66 (dd, J=4.4, 1.6 Hz, 2H), 7.93 (d, J=2.4 Hz, 1H), 7.87 (d, J=2.4 Hz, 1H), 7.83 (dd, J=4.4, 1.6 Hz, 2H), 7.48 (d, J=3.6 Hz, 1H), 7.43 (d, J=3.6 Hz, 1H), 4.95-4.73 (m, 1H), 3.26-3.19 (m, 2H), 2.96-2.86 (m, 2H), 2.00-1.87 (m, 6H), 1.82-1.67 (m, 2H), 0.93-0.91 (m, 2H), 0.67-0.60 (m, 2H).

LCMS (ESI) m/z: [M+H]⁺ 447.1; Purity=95% (254 nm); Retention time=1.35 min.

### Example 6: Preparation of N-(2-((2R,4S)-4-hydroxy-2-methylpiperidin-1-yl)-5-(trifluoromethyl)pyridin-3-yl)-5-(pyridin-4-yl) furan-2-carboxamide (Compound 079) and N-(2-((2S,4S)-4-hydroxy-2-methylpiperidin-1-yl)-5-(trifluoromethyl)pyridin-3-yl)-5-(pyridin-4-yl) furan-2-carboxamide (Compound 080)

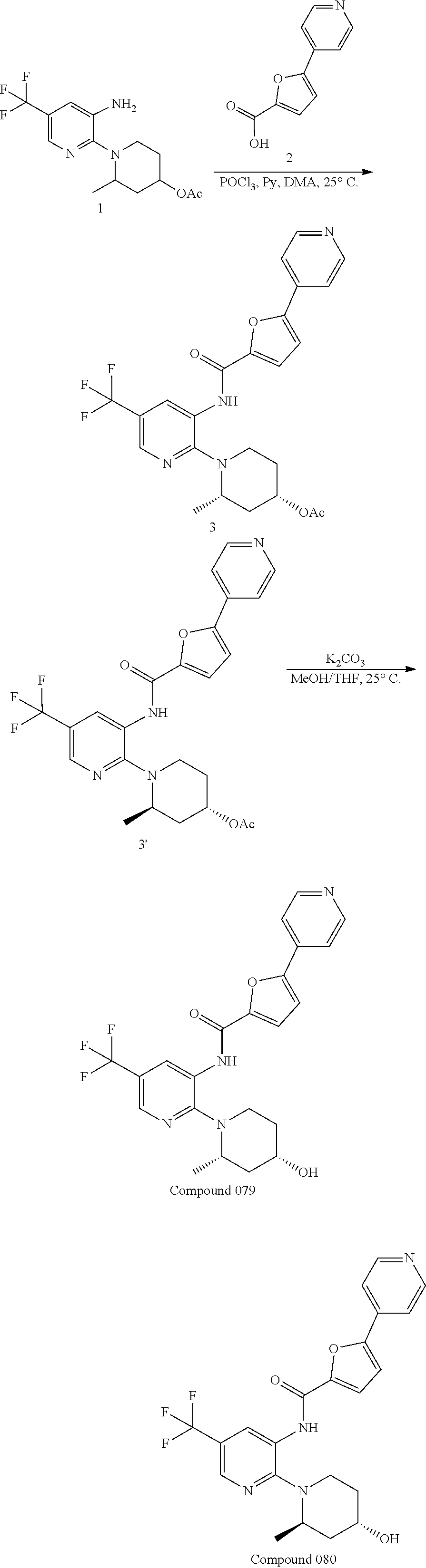

Step-1. To a solution of 5-(4-pyridyl) furan-2-carboxylic acid (119.23 mg, 0.6 mmol) and [1-[3-amino-5-(trifluoromethyl)-2-pyridyl]-2-methyl-4-piperidyl]acetate (200 mg, 0.6 mmol) in N,N-dimethylacetamide (1 mL) was added pyridine (2.49 g, 31.52 mmol, 2.54 mL). The mixture was stirred at 0° C. for 10 minutes. Then phosphoryl trichloride (1.93 g, 12.61 mmol, 1.18 mL) was added at 0° C. The mixture was stirred at 25° C. for 30 mins. The reaction mixture was diluted with ethyl acetate (50 mL) and washed with water (30 mL×3) and brine (30 mL×3). The organic layer was dried over anhydrous sodium sulfate, filtered and concentrated under reduced pressure. The crude product was purified by silica column chromatography (dichloromethane:methanol=25:1) to give (2S,4S)-2-methyl-1-(3-(5-(pyridin-4-yl) furan-2-carboxamido)-5-(trifluoromethyl)pyridin-2-yl) piperidin-4-yl acetate and (2R,4S)-2-methyl-1-(3-(5-(pyridin-4-yl) furan-2-carboxamido)-5-(trifluoromethyl)pyridin-2-yl) piperidin-4-yl acetate.

Step-2. To a solution of (2S,4S)-2-methyl-1-(3-(5-(pyridin-4-yl) furan-2-carboxamido)-5-(trifluoromethyl)pyridin-2-yl) piperidin-4-yl acetate (40 mg, 0.08 mmol) in tetrahydrofuran (2 mL) and methanol (2 mL) was added dipotassium carbonate (33.95 mg, 0.24 mmol). The mixture was stirred at 25° C. for 6 hours. LCMS analysis showed target product formed. The reaction mixture was diluted with ethyl acetate (50 mL) and washed with water (30 mL×3) and brine (30 mL×3). The organic layer was dried over anhydrous sodium sulfate, filtered and concentrated under reduced pressure. The crude product was purified by silica column chromatography (dichloromethane:methanol=20:1) to give N-(2-((2S,4S)-4-hydroxy-2-methylpiperidin-1-yl)-5-(trifluoromethyl)pyridin-3-yl)-5-(pyridin-4-yl) furan-2-carboxamide.

¹HNMR (400 MHz, DMSO-_{d6}) δ 9.97 (s, 1H), 8.80 (d, J=2.0 Hz, 1H), 8.72 (d, J=4.8 Hz, 1H), 8.58 (s, 1H), 7.89 (d, J=4.4 Hz, 2H), 7.58 (d, J=4.0 Hz, 1H), 7.53 (d, J=3.6 Hz, 2H), 4.94 (d, J=3.6 Hz, 1H), 3.79-3.70 (m, 1H), 3.48-3.41 (m, 1H), 3.13-3.08 (m, 1H), 2.87-2.80 (m, 1H), 2.08-2.05 (m, 1H), 1.98-1.94 (m, 1H), 1.70-1.61 (m, 1H), 1.54-1.46 (m, 1H), 0.94 (d, J=3.6 Hz, 3H).

LCMS (ESI) m/z: [M+H]⁺ 447.19; Purity=97.25% (254 nm); Retention time=2.421 min.

Step-3. To a solution of (2R,4S)-2-methyl-1-(3-(5-(pyridin-4-yl) furan-2-carboxamido)-5-(trifluoromethyl)pyridin-2-yl) piperidin-4-yl acetate (15 mg, 0.03 mmol) in tetrahydrofuran (2 mL) and methanol (2 mL) was added dipotassium carbonate (12.73 mg, 0.09 mmol). The mixture was stirred at 25° C. for 6 hours. LCMS analysis showed target product formed. The reaction mixture was diluted with ethyl acetate (50 mL) and washed with water (30 mL×3) and brine (30 mL×3). The organic layer was dried over anhydrous sodium sulfate, filtered and concentrated under reduced pressure. The crude product was purified by silica column chromatography (dichloromethane:methanol=20:1) to give N-(2-((2R,4S)-4-hydroxy-2-methylpiperidin-1-yl)-5-(trifluoromethyl)pyridin-3-yl)-5-(pyridin-4-yl) furan-2-carboxamide.

¹HNMR (400 MHz, DMSO-_{d6}) δ 9.98 (s, 1H), 8.70 (d, J=6.0 Hz, 2H), 8.48 (d, J=1.6 Hz, 1H), 8.24 (d, J=2.0 Hz, 1H), 7.89 (d, J=6.0 Hz, 2H), 7.52 (d, J=3.6 Hz, 1H), 7.48 (d, J=4.0 Hz, 1H), 4.63 (d, J=4.4 Hz, 1H), 4.22-4.18 (m, 1H), 3.93-3.86 (m, 1H), 3.69-3.64 (m, 1H), 3.13-3.06 (m, 1H), 1.85-1.81 (m, 1H), 1.74-1.69 (m, 1H), 1.60-1.50 (m, 2H), 1.11 (d, J=6.8 Hz, 3H).

LCMS (ESI) m/z: [M+H]⁺ 447.19; Purity=99.62% (254 nm); Retention time=2.316 min.

The following compound were prepared in a similar manner to the procedure described above.

| | LCMS | |
|---|---|---|
| Compound | (ESI): | |
| No. | [M + H]⁺ | ¹H NMR |
| 075 | 454.24 | ¹H NMR (400 MHz, DMSO-d₆) δ 9.65 (s, 1H), 8.82 (d, J = 2.4 Hz, |
| | | 1H), 8.52 (s, 1H), 7.26 (d, J = 3.2 Hz, 1H), 6.46 (d, J = 3.2 Hz, 1H), |
| | | 4.84 (s, 1H), 3.94-3.91 (m, 2H), 3.71-3.64 (m, 1H), 3.50-3.44 |
| | | (m, 2H), 3.40-3.35 (m, 1H), 3.10-3.01 (m, 2H), 2.78-2.74 (m, |
| | | 1H), 2.03-1.92 (m, 4H), 1.77-1.65 (m, 2H), 1.61-1.51 (m, 1H), |
| | | 1.39-1.31 (m, 1H), 0.87 (d, J = 6.0 Hz, 3H). |
| 076 | 454.23 | ¹H NMR (400 MHz, DMSO-d₆) δ 9.57 (s, 1H), 8.41-8.39 (m, |
| | | 2H), 7.20 (s, 1H), 6.39 (d, J = 2.8 Hz, 1H), 4.63 (s, 1H), 4.13 (s, |
| | | 1H), 3.92-3.89 (m, 3H), 3.60-3.51 (m, 1H), 3.48-3.42 (m, 2H), |
| | | 3.04-2.99 (m, 2H), 2.03-1.82 (m, 4H), 1.73-1.62 (m, 3H), |
| | | 1.58-1.53 (m, 1H), 1.02 (d, J = 6.8 Hz, 3H). |

### Example 7: Preparation of 4-hydroxy-1-[3-[(5-tetrahydropyran-4-ylfuran-2-carbonyl)amino]-5-(trifluoromethyl)-2-pyridyl]piperidine-4-carboxamide (Compound 096)

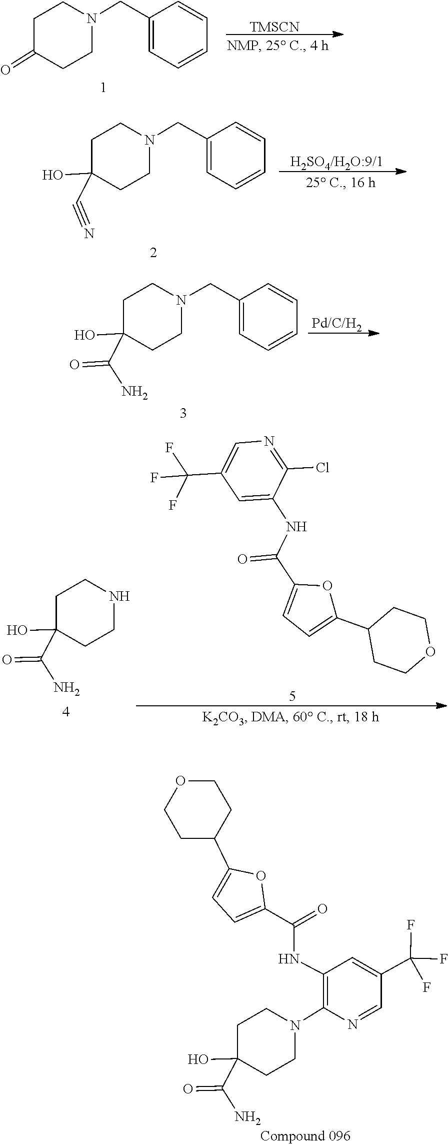

Step-1. To a mixture of 1-benzylpiperidin-4-one (4.00 g, 21.14 mmol) in N-Methyl-2-pyrrolidone (40 mL), and the Trimethylsilyl cyanide (4.19 g, 42.27 mmol) was dropwised into the mixture at 25° C. The mixture was stirred for 4 hours at 25° C. TLC (petroleum ether:ethyl acetate=10:1, Rf=0.35) showed 1-benzylpiperidin-4-one was consumed and a new point was appeared. To the mixture was added water (20 mL) and extracted with ethyl acetate (20 mL×3). The organic layer was dried over sodium sulfate (25 g), filtered and concentrated under reduced pressure to give residue. The residue was purified by column chromatography on silica gel (petroleum ether:ethyl acetate=50:1~ 20:1) to afford the title compound 1-benzyl-4-hydroxy-piperidine-4-carbonitrile.

Step-2. To a solution of 1-benzyl-4-hydroxy-piperidine-4-carbonitrile (2.00 g, 9.25 mmol) was added in sulfuric acid:water (8 mL, v/v=9:1) at 0° C. The reaction mixture was stirred at 25° C. for 16 h. LCMS showed target product formed. The mixture was poured into water (10 mL). Then pH value was adjusted to 6.0 with aqueous sodium bicarbonate solution (150 mL) and concentrated in vacuum. The solid was washed with dichloromethane:methanol (5:1) and concentrated in vacuum to afforded 1-benzyl-4-hydroxy-piperidine-4-carboxamide.

Step-3. To a solution of 1-benzyl-4-hydroxy-piperidine-4-carboxamide (950 mg, 4.05 mmol) in methanol (10 mL), added palladium (431 mg, 4.05 mmol, 10% Pd, wet). The reaction mixture was stirred at 25° C. for 16 h under hydrogen. TLC showed the material was consumed completely. The mixture was filtered through a 6-cm fritted glass funnel and concentrated in vacuum to afford the target product 4-hydroxypiperidine-4-carboxamide.

Step-4. A solution of N-[2-chloro-5-(trifluoromethyl)-3-pyridyl]-5-tetrahydropyran-4-yl-furan-2-carboxamide (100 mg, 0.27 mmol), 4-hydroxypiperidine-4-carboxamide (77 mg, 0.53 mmol) and Potassium carbonate (111 mg, 0.80 mmol) in N, N-Dimethylacetamide (4 mL) was stirred at 25° C. for 2 hours. LCMS showed target product formed. The mixture was filtered through a 6-cm fritted glass funnel, added water (30 mL) in the filtrate. The combined aqueous layers are extracted with ethyl acetate (3×10 mL) and the combined organic layers washed with water (3×10 mL), dried with brine (2×20 mL), sodium sulfate (10 g) and concentrated in vacuum. The crude product was purified by prepare TLC (Dichloromethane/Methanol=20:1) to afford the target compound 4-hydroxy-1-[3-[(5-tetrahydropyran-4-ylfuran-2-carbonyl)amino]-5-(trifluoromethyl)-2-pyridyl]piperidine-4-carboxamide.

¹HNMR (400 MHz, DMSO-_{d6}) δ 9.36 (s, 1H), 8.44 (d, J=2.4 Hz, 1H), 8.37 (d, J=2.4 Hz, 1H), 7.28 (d, J=2.0 Hz, 1H), 7.22 (d, J=1.2 Hz, 1H), 7.10 (d, J=1.6 Hz, 1H), 6.42 (d, J=4.8 Hz, 1H), 5.39 (s, 1H), 3.93-3.90 (m, 2H), 3.55-3.44 (m, 4H), 3.18-3.15 (m, 2H), 3.12-3.03 (m, 1H), 2.16-2.09 (m, 2H), 1.96-1.92 (m, 2H), 1.67-1.55 (m, 4H).

LCMS (ESI) m/z: [M+H]⁺ 483.27; Purity=99% (254 nm); Retention time=2.37 min.

The following compound were prepared in a similar manner to the procedure described above.

| | LCMS | |
|---|---|---|
| Compound | (ESI): | |
| No. | [M + H]⁺ | ¹H NMR |
| 035 | 426.1 | ¹H NMR (400 MHz, DMSO-d₆) δ 9.90 (s, 1H), 8.31 (dd, J = 2.4, 1.2 |
| | | Hz, 1H), 7.61 (d, J = 2.4 Hz, 1H), 7.17 (d, J = 3.6 Hz, 1H), 6.33 (dd, |
| | | J = 3.6, 0.8 Hz, 1H), 4.91 (d, J = 3.2 Hz, 1H), 4.25 (s, 1H), 3.91- |
| | | 3.84 (m, 2H), 3.68-3.54 (m, 3H), 3.45-3.35 (m, 3H), 3.00-2.90 |
| | | (m, 1H), 1.90-1.82 (m, 3H), 1.80-1.72 (m, 1H), 1.69-1.57 (m, |
| | | 2H). |
| 036 | 424.5 | ¹H NMR (400 MHz, DMSO-d₆) δ11.71 (s, 1H), 7.30 (s, 1H), 6.48 |
| | | (d, J = 2.8 Hz, 1H), 6.30 (d, J = 3.6 Hz, 1H), 6.07 (dd, J = 3.6, 1.2 |
| | | Hz, 1H), 3.80-3.71 (m, 2H), 3.37-3.30 (m, 6H), 2.83-2.70 (m, |
| | | 1H), 1.70-1.50 (m, 8H), 1.45-1.34 (m, 2H). |
| 037 | 426.5 | ¹H NMR (400 MHz, DMSO-d₆) δ 11.81 (s, 1H), 7.38 (s, 1H), 6.61 |
| | | (d, J = 2.4 Hz, 1H), 6.43 (d, J = 4.0 Hz, 1H), 6.14 (d, J = 4.0 Hz, |
| | | 1H), 3.81-3.76 (m, 2H), 3.68-3.66 (m, 4H), 3.41-3.33 (m, 6H), |
| | | 2.83-2.76 (m, 1H), 1.69-1.65 (m, 2H), 1.46-1.36 (m, 2H). |
| 038 | 440.1 | ¹H NMR (400 MHz, DMSO-d₆) δ 9.90 (s, 1H), 8.34-8.26 (s, 1H), |
| | | 7.63 (s, 1H), 7.17 (d, J = 4.0 Hz, 1H), 6.32 (d, J = 4.0 Hz, 1H), 4.64 |
| | | (t, J = 6.0 Hz, 1H), 3.89-3.86 (m, 2H), 3.67-3.33 (m, 11H), 1.96- |
| | | 1.78 (m, 3H), 1.68-1.55 (m, 2H). |
| 039 | 412.0 | ¹H NMR (400 MHz, DMSO-d₆) δ 9.79 (s, 1H), 8.33 (dd, J = 2.0, 1.2 |
| | | Hz, 1H), 7.72-7.68 (m, 1H), 7.22 (d, J = 4.0 Hz, 1H), 6.37 (dd, J = |
| | | 4.0, 0.8 Hz, 1H), 5.62 (d, J = 8.0 Hz, 1H), 4.54-4.46 (m, 1H), 4.31- |
| | | 4.27 (m, 2H), 3.95-3.81 (m, 4H), 3.47-3.44 (m, 2H), 3.02-2.96 |
| | | (m, 1H), 1.92-1.88 (m, 2H), 1.74-1.60 (m, 2H). |
| 040 | 495.1 | ¹H NMR (400 MHz, DMSO-d₆) δ 9.37 (s, 1H), 8.44 (dd, J = 4.0 Hz, |
| | | 1H), 8.37 (d, J = 4.0 Hz, 1H), 7.28 (d, J = 4.0 Hz, 1H), 6.42 (dd, J = |
| | | 4.0 Hz, 1H), 3.93-3.90 (m, 2H), 3.90-3.49 (m, 2H), 3.47-3.31 (m, |
| | | 2H), 3.04-2.99 (m, 4H), 2.95-2.91 (m, 3H), 2.89-2.82 (m, 3H), |
| | | 1.96-1.92 (m, 2H),1.80-1.61 (m, 6H). |
| 041 | 442.1 | ¹H NMR (400 MHz, DMSO-d₆) δ 11.76 (s, 1H), 7.34 (s, 1H), 6.56 |
| | | (d, J = 2.4 Hz, 1H), 6.37 (d, J = 3.2 Hz, 1H), 6.09 (d, J = 3.2, 0.8 Hz, |
| | | 1H), 3.77-3.74 (m, 2H), 3.67-3.65 (m, 4H), 3.36-3.31 (m, 2H), |
| | | 2.80-2.73 (m, 1H), 2.65-2.63 (m, 4H), 1.68-1.65 (m, 2H), 1.44- |
| | | 1.34 (m, 2H). |
| 042 | 474.2 | ¹H NMR (400 MHz, DMSO-d₆) δ 11.84 (s, 1H), 7.40 (s, 1H), 6.74 |
| | | (d, J = 2.4 Hz, 1H), 6.49 (d, J = 3.2 Hz, 1H), 6.13 (d, J = 3.2, 0.8 Hz, |
| | | 1H), 3.82-3.80 (m, 4H), 3.78-3.73 (m, 2H), 3.36-3.30 (m, 2H), |
| | | 3.22-3.19 (m, 4H), 2.81-2.73 (m, 1H), 1.67-1.63 (m, 2H) 1.44- |
| | | 1.34 (m, 2H) |
| 043 | 467.1 | ¹H NMR (400 MHz, CDCl₃) δ 8.75-8.53 (m, 2H), 7.29-7.27 (m, |
| | | 2H), 7.10 (d, J = 8.0 Hz, 1H), 7.03 (d, J = 8.0 Hz, 1H), 6.82 (d, J = |
| | | 3.6 Hz, 1H), 6.53 (d, J = 3.6 Hz, 1H), 4.07 (brs, 2H), 3.59-3.49 (m, |
| | | 2H), 1.82-1.66 (m, 4H), 1.36 (s, 3H). |
| 045 | 509.1 | ¹H NMR (400 MHz, DMSO-d₆) δ 9.34 (s, 1H), 8.38 (s, 1H), 8.30 (s, |
| | | 1H), 7.26 (d, J = 8.0 Hz, 1H), 6.39 (d, J = 4.0 Hz, 1H), 3.91 (dd, J = |
| | | 8.0, 12.0 Hz, 2H), 3.46-3.41 (m, 2H), 3.36-3.34 (m, 3H), 3.16- |
| | | 2.93 (m, 2H), 2.99-2.93 (m, 6H), 2.20-2.16 (m, 2H), 1.89-1.87 (m, |
| | | 2H), 1.70-1.60 (m, 4H), 1.23 (s, 3H). |
| 048 | 481.1 | ¹H NMR (400 MHz, DMSO-d₆) δ 9.35 (s, 1H), 8.42 (s, 1H), 8.36 (s, |
| | | 1H), 7.28-7.21 (m, 2H), 6.94 (s, 1H), 6.43 (d, J = 4.0 Hz, 1H), |
| | | 3.94-3.90 (m, 2H), 3.49-3.36 (m, 4H), 3.08-3.00 (m, 3H), 2.14-2.10 |
| | | (m, 2H), 1.93-1.90 (m, 2H), 1.73-1.64 (m, 2H), 1.55-1.49 (m, |
| | | 2H), 1.14 (s, 3H). |
| 052 | 366.4 | ¹H NMR (400 MHz, DMSO-d₆) δ 9.41 (s, 1H), 8.42 (d, J = 1.2 Hz, |
| | | 1H), 8.34(d, J = 4.0 Hz, 1H), 7.28 (d, J = 2.4 Hz, 1H), 6.42 (d, J = |
| | | 4.0 Hz, 1H), 4.35 (s, 4H), 3.93-3.83 (m, 2H), 3.47-3.42 (m, 2H), |
| | | 3.20-3.18 (m, 4H), 3.17-3.02 (m, 1H), 1.95-1.87 (m, 6H), 1.74- |
| | | 1.67 (m, 2H). |
| 055 | 468.2 | ¹H NMR (400 MHz, DMSO-d₆) δ 9.83 (s, 1H), 8.35 (dd, J = 4.0, 1.2 |
| | | Hz, 1H), 7.74 (d, J = 4.0 Hz, 1H), 7.19 (d, J = 4.0 Hz, 1H), 6.36 (dd, |
| | | J = 4.0, 0.8 Hz, 1H), 4.24 (s, 1H), 3.94-3.87 (m, 2H), 3.65-3.39 |
| | | (m, 6H), 3.03-2.93 (m, 1H), 2.09-1.51 (m, 10H), 1.09 (s, 3H). |
| 057 | 495.1 | ¹H NMR (400 MHz, DMSO-d₆) δ 9.32 (s, 1H), 8.38-8.37 (m, 1H), |
| | | 8.32-8.31 (m, 1H), 7.60-7.56 (m, 1H), 7.24 (d, J = 3.6 Hz, 1H), |
| | | 6.40-6.38 (m, 1H), 3.89-3.86 (m, 2H), 3.38-3.34 (m, 4H), 3.02- |
| | | 2.97 (m, 3H), 2.58 (d, J = 4.4 Hz, 3H), 2.11-2.08 (m, 2H), 1.87-1.88 |
| | | (m, 2H), 1.69-1.61 (m, 2H), 1.52-1.47 (m, 2H), 1.09 (s, 3H). |
| 072 | 440.3 | ¹H NMR (400 MHz, DMSO-d₆) δ 9.48 (s, 1H), 8.43-8.38 (m, 1H), |
| | | 8.32 (d, J = 2.4 Hz, 1H), 7.32 (d, J = 4.0 Hz, 1H), 6.40 (d, J = 4.0 |
| | | Hz, 1H), 4.99-4.90 (m, 1H), 3.95-3.86 (m, 2H), 3.70 (s, 1H), 3.58- |
| | | 3.37 (m, 4H), 3.06-2.95 (m, 2H), 2.89-2.82 (m, 1H), 2.04-1.77 |
| | | (m, 4H), 1.75-1.38 (m, 4H). |
| 073 | 493.2 | ¹H NMR (400 MHz, DMSO-d₆) δ 9.36 (s, 1H), 8.44 (dd, J = 1.2 Hz, |
| | | 3.6 Hz, 1H), 8.39 (d, J = 2.4 Hz, 1H), 7.59 (s, 1H), 7.27 (d, J = 3.6 |
| | | Hz, 1H), 6.41 (dd, J = 1.2 Hz, 4.4 Hz, 1H), 3.97-3.84 (m, 2H), |
| | | 3.67-3.55 (m, 2H), 3.49-3.44 (m, 2H), 3.19 (t, J = 6.8 Hz, 2H), |
| | | 3.09-2.91 (m, 3H), 2.08-1.81 (m, 6H), 1.73-1.58 (m, 2H), 1.48-1.47 |
| | | (m, 2H). |
| 078 | 462.2 | ¹H NMR (400 MHz, DMSO-d₆) δ 12.51 (brs, 1H), 9.72 (brs, 1H), |
| | | 8.44 (s, 1H), 8.18 (s, 1H), 7.44 (brs, 1H), 7.29 (s, 1H), 6.40 (s, 1H), |
| | | 4.40 (s, 2H), 3.91-3.89 (m, 2H), 3.59 (s, 2H), 3.50-3.43 (m, 2H), |
| | | 3.02-2.92 (m, 1H), 2.82 (s, 2H), 1.87-1.85 (m, 2H), 1.66-1.64 |
| | | (m, 2H). |
| 081 | 493.2 | ¹H NMR (400 MHz, DMSO-d₆) δ 9.45 (s, 1H), 8.43 (dd, J = 4.4, 1.2 |
| | | Hz, 1H), 8.25 (d, J = 2.4 Hz, 1H), 8.09 (s, 1H), 7.27 (d, J = 3.2 Hz, |
| | | 1H), 6.41 (dd, J = 3.6, 0.8 Hz, 1H), 3.99-3.86 (m, 2H), 3.54-3.39 |
| | | (m, 4H), 3.34-3.33 (m, 2H), 3.07-2.99 (m, 1H), 2.23-2.19 (m, |
| | | 2H), 1.97-1.82 (m, 4H), 1.73-1.60 (m, 6H). |
| 082 | 494.2 | ¹H NMR (400 MHz, DMSO-d₆) δ 9.40 (s, 1H), 8.45 (s, 1H), 8.39 |
| | | (d, J = 2.4 Hz, 1H), 7.28 (d, J = 3.6 Hz, 1H), 6.43 (d, J = 2.8 Hz, |
| | | 1H), 4.31 (t, J = 6.8 Hz, 2H), 3.92 (dd, J = 11.6, 2.0 Hz, 2H), 3.62 |
| | | (d, J = 13.2 Hz, 2H), 3.51-3.41 (m, 2H), 3.02 (t, J = 12.0 Hz, 3H), |
| | | 2.26 (t, J = 7.2 Hz, 2H), 1.99-1.87 (m, 4H), 1.76-1.61 (m, 4H). |
| 088 | 467.2 | ¹H NMR (400 MHz, DMSO-d₆) δ 9.35 (s, 1H), 8.44 (d, J = 1.2 Hz, |
| | | 1H), 8.39 (d, J = 2.4 Hz, 1H), 7.34-7.23 (m, 2H), 6.81 (s, 1H), |
| | | 6.47-6.39 (m, 1H), 3.98-3.87 (m, 2H), 3.73-3.63 (m, 2H), 3.53- |
| | | 3.42 (m, 2H), 3.09-2.98 (m, 1H), 2.91-2.79 (m, 2H), 2.39-2.26 |
| | | (m, 1H), 2.01-1.90 (m, 2H), 1.88-1.60 (m, 6H). |

### Example 8: Preparation of N-(2-(4-hydroxypiperidin-1-yl)-5-(trifluoromethyl)pyridin-3-yl)-5-(pyrazin-2-yl) furan-2-carboxamide (Compound 074)

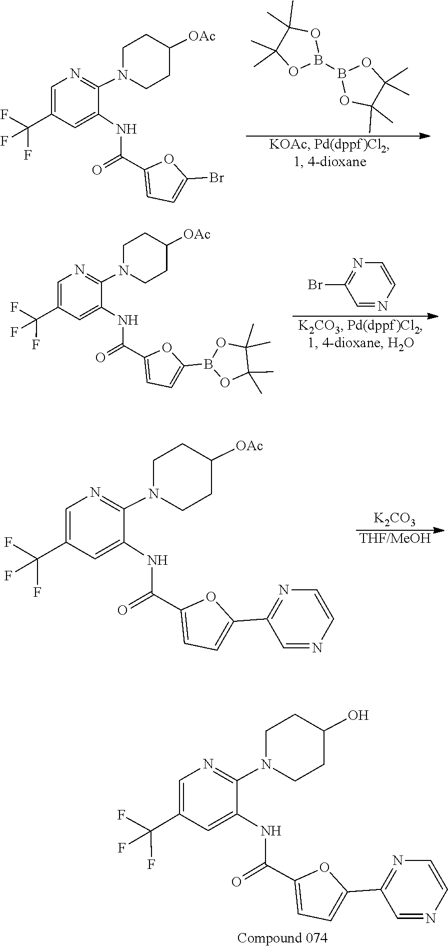

Step-1. To a solution of [1-[3-[(5-bromofuran-2-carbonyl)amino]-5-(trifluoromethyl)-2-pyridyl]-4-piperidyl]acetate (50 mg, 0.10 mmol) in 1,4-dioxane (3 mL). Added 4,4,5,5-tetramethyl-2-(4,4,5,5-tetramethyl-1,3,2-dioxaborolan-2-yl)-1,3,2-dioxaborolane (40 mg, 0.16 mmol), potassium acetate (31 mg, 0.31 mmol) and 1,1′-Bis(diphenylphosphino) ferrocene-palladium (II) dichloride dichloromethane complex (8 mg, 0.01 mmol). After deoxygenating the flask with three alternating vacuum and purge cycles, the reaction mixture was stirred at 105° C. for 2 hours. LCMS analysis showed the orthoboric acid mass. The mixture was filtered through a 6-cm fritted glass funnel and concentrated in vacuum to afford the target compound [1-[3-[[5-(4,4,5,5-tetramethyl-1,3,2-dioxaborolan-2-yl) furan-2-carbonyl]amino]-5-(trifluoromethyl)-2-pyridyl]-4-piperidyl]acetate.

Step-2. To a solution of [1-[3-[[5-(4,4,5,5-tetramethyl-1,3,2-dioxaborolan-2-yl) furan-2-carbonyl]amino]-5-(trifluoromethyl)-2-pyridyl]-4-piperidyl]acetate (20 mg, 0.04 mmol) and 2-bromopyrazine (6 mg, 0.04 mmol) in 1,4-dioxane (3 mL). Added potassium carbonate (16 mg, 0.12 mmol), [1,1′-Bis(diphenylphosphino) ferrocene]dichloropalladium (II) (3 mg, 0.04 mmol) and water (0.3 mL). After deoxygenating the flask with three alternating vacuum and purge cycles, the reaction mixture was stirred at 105° C. for 16 hours. LCMS analysis showed the target product was formed. The mixture was filtered through a 6-cm fritted glass funnel, added water (50 mL) in the filtrate. The combined aqueous layers are extracted with ethyl acetate (3×20 mL) and the combined organic layers washed with water (3×50 mL), dried with brine (2×50 mL), sodium sulfate (10 g) and concentrated in vacuum. The crude material obtained as the residue was purified by prep-TLC (dichloromethane/methanol=17:1) to afford the target compound [1-[3-[(5-pyrazin-2-ylfuran-2-carbonyl)amino]-5-(trifluoromethyl)-2-pyridyl]-4-piperidyl]acetate.

Step-3. To a solution of [1-[3-[(5-pyrazin-2-ylfuran-2-carbonyl)amino]-5-(trifluoromethyl)-2-pyridyl]-4-piperidyl]acetate (10 mg, 0.02 mmol) in methanol (1 mL) and tetrahydrofuran (1 mL). Added potassium carbonate (9 mg, 0.06 mmol), the reaction mixture was stirred at 25° C. for 2 hours. LCMS analysis indicated the target product formed. The mixture was filtered through a 6-cm fritted glass funnel, added water (20 mL) in the filtrate. The combined aqueous layers are extracted with ethyl acetate (3×15 mL) and the combined organic layers washed with water (3×20 mL), dried with brine (2×20 mL), sodium sulfate (10 g) and concentrated in vacuum. The crude material obtained as the residue was purified by prepare TLC (dichloromethane/methanol=17:1) to afford the target compound N-[2-(4-hydroxy-1-piperidyl)-5-(trifluoromethyl)-3-pyridyl]-5-pyrazin-2-yl-furan-2-carboxamide.

¹HNMR (400 MHz, DMSO-_{d6}) δ 10.09 (s, 1H), 9.34 (s, 1H), 8.73 (dd, J=2.4, 1.6 Hz, 1H), 8.65 (d, J=4.0 Hz, 1H), 8.47-8.41 (m, 1H), 8.16 (d, J=2.4 Hz, 1H), 7.52 (d, J=4.0 Hz, 1H), 7.46 (d, J=4.0 Hz, 1H), 4.73 (d, J=4.0 Hz, 1H), 3.77-3.64 (m, 3H), 3.13-3.01 (m, 2H), 1.88-1.78 (m, 2H), 1.60-1.47 (m, 2H).

LCMS (ESI) m/z: [M+H]⁺ 476.20; Purity=100.00% (254 nm); Retention time=2.63 min.

### Example 9: Preparation of N-[2-(4-hydroxy-1-piperidyl)-5-(trifluoromethyl)-3-pyridyl]-4-(1H-pyrazol-4-yl)pyridine-2-carboxamide (Compound 094)

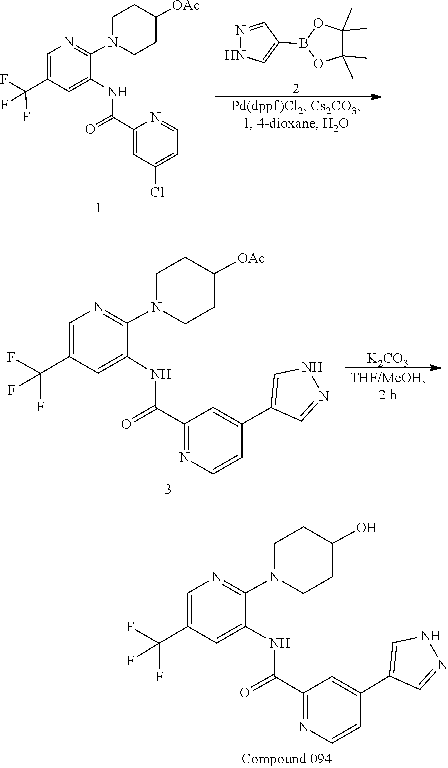

Step-1. A solution of 1-(3-(4-chloropicolinamido)-5-(trifluoromethyl)pyridin-2-yl) piperidin-4-yl acetate (90 mg, 0.2 mmol) and 4-(4,4,5,5-tetramethyl-1,3,2-dioxaborolan-2-yl)-1H-pyrazole (120 mg, 0.4 mmol) in 1,4-dioxane (15 mL) and water (1.5 mL) was added cesium carbonate (198 mg, 0.1 mmol) and [1,1′-Bis(diphenylphosphino) ferrocene] dichloropalladium (II) (2.88 mg, 0.004 mmol). The mixture solution was stirred at 100° C. for 16 h under nitrogen atmosphere. The reaction was monitored by TLC, it showed the starting material was consumed up and the desire product formed, extracted with ethyl acetate (100 mL) and water (150 mL). The combined organic layer was dried over anhydrous sodium sulfate, filtered, and concentrated to give a residue and purified by flash chromatography column with eluting PE:EtOAc=1:1 to give 1-(3-(4-(1H-pyrazol-4-yl) picolinamido)-5-(trifluoromethyl)pyridin-2-yl) piperidin-4-yl acetate.

Step-2. A solution of [1-[3-[4-(1H-pyrazol-4-yl)pyridine-2-carbonyl]amino]-5-(trifluoromethyl)-2-pyridyl]-4-piperidyl]acetate (40 mg, 0.084 mmol) and potassium carbonate (35 mg, 0.25 mmol) in Tetrahydrofuran (1 mL) and Methanol (1 mL) was stirred at 25° C. for 2 hrs. LCMS analysis showed target product formed. The mixture was quenched with water (25 mL). The combined aqueous layers are extracted with ethyl acetate (3×20 mL), and the combined organic layers are dried with brine (2×25 mL), sodium sulfate (10 g) and concentrated in vacuum. The crude product was purified by prep-HPLC to afford the target compound N-[2-(4-hydroxy-1-piperidyl)-5-(trifluoromethyl)-3-pyridyl]-4-(1H-pyrazol-4-yl) pyridine-2-carboxamide.

¹HNMR (400 MHz, DMSO-_{d6}) δ 13.29 (s, 1H), 10.52 (s, 1H), 8.87 (d, J=2.4 Hz, 1H), 8.65 (d, J=5.6 Hz, 1H), 8.45-8.38 (m, 4H), 7.96-7.94 (m, 1H), 4.78 (s, 1H), 3.76-3.71 (m, 1H), 3.46-3.41 (m, 2H), 3.00-2.94 (m, 2H), 1.97-1.93 (m, 2H), 1.74-1.68 (m, 2H).

LCMS (ESI) m/z: [M+H]⁺ 433.26; Purity=100% (254 nm); Retention time=2.40 min.

The following compound was prepared in a similar manner to the procedure described above.

| | LCMS | |
|---|---|---|
| Compound | (ESI): | |
| No. | [M + H]⁺ | ¹H NMR |
| 100 | 433.21 | ¹H NMR (400 MHz, CDCl₃) δ 13.24 (s, 1H), 10.50 (s, 1H), 8.74 |
| | | (d, J = 2.8 Hz, 1H), 8.48 (d, J = 2.4 Hz, 1H), 8.27 (s, 1H), 8.09- |
| | | 7.96 (m, 3H), 7.34-7.11 (m, 3H), 4.70 (d, J = 3.2 Hz, 1H), |
| | | 3.73-3.70 (m, 1H), 3.49-3.43 (m, 2H), 3.05-2.99 (m, 2H), 1.91- |
| | | 1.86 (m, 2H), 1.65-1.58 (m, 2H). |

### Example 10: Preparation of N-[5-cyclopropyl-2-(4-hydroxy-4-methyl-1-piperidyl)-3-pyridyl]-5-(1H-pyrazol-4-yl) furan-2-carboxamide (Compound 095)

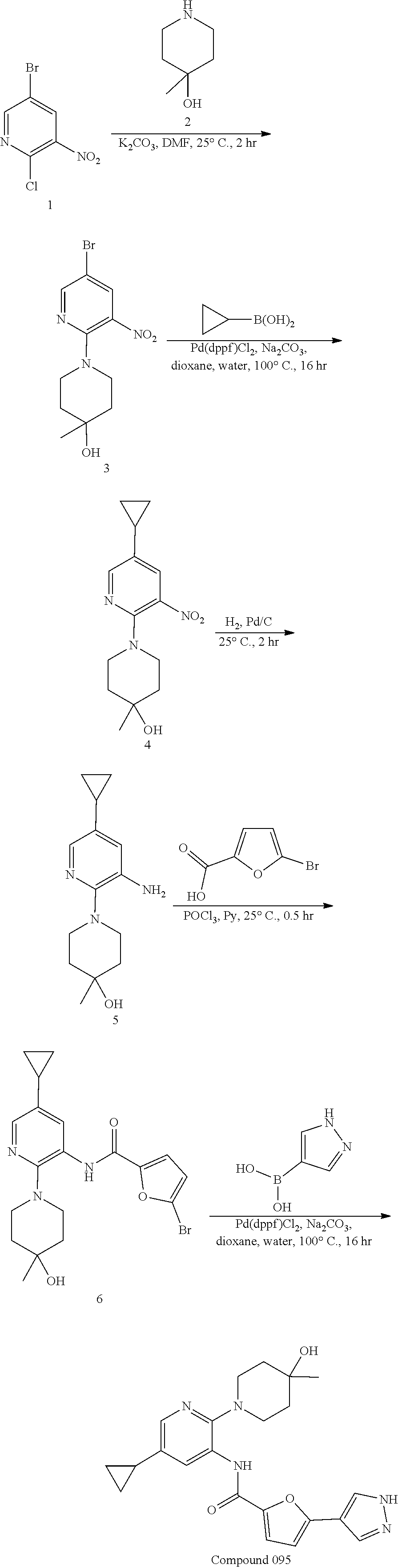

Step-1. To a solution of 5-bromo-2-chloro-3-nitro-pyridine (5 g, 21.09 mmol) and 4-methylpiperidin-4-ol (2.43 g, 21.09 mmol) in N,N-dimethylformamide (40 mL). Added potassium carbonate (8.73 g, 63.27 mmol), the reaction mixture was stirred at 25° C. for 2 hrs. TLC analysis indicated the total consumption of the starting material. The mixture was filtered through a 6-cm fritted glass funnel, added water (150 mL) in the filtrate. The combined aqueous layers are extracted with ethyl acetate (3×100 mL) and the combined organic layers washed with water (3×100 mL), dried with brine (2×100 mL), sodium sulfate (150 g) and concentrated in vacuum. The crude material obtained as the residue was purified by column chromatography with a column containing 80 g of silica gel, the column is eluted with a mixture of petroleum ether and ethyl acetate (10:1) to afford the target compound 1-(5-bromo-3-nitro-2-pyridyl) piperidin-4-ol.

Step-2. To a solution of 1-(5-bromo-3-nitro-2-pyridyl)-4-methyl-piperidin-4-ol (5.87 g, 18.57 mmol), sodium carbonate (5.90 g, 55.70 mmol, 2.33 mL), [1,1′-Bis(diphenylphosphino) ferrocene]dichloropalladium (II) (1.36 g, 1.86 mmol) and cyclopropylboronic acid (1.91 g, 22.28 mmol) in 1,4-dioxane (60 mL) and water (10 mL). After deoxygenating the flask with three alternating vacuum and purge cycles, the reaction mixture was stirred at 100° C. for 16 hrs. LCMS analysis showed completed consumption of 1-(5-bromo-3-nitro-2-pyridyl)-4-methyl-piperidin-4-ol, the target product was formed. The mixture was filtered through a 6-cm fritted glass funnel, added water (150 mL) in the filtrate. The combined aqueous layers are extracted with ethyl acetate (3×100 mL) and the combined organic layers washed with water (3×100 mL), dried with brine (2×100 mL), sodium sulfate (150 g) and concentrated in vacuum. The crude material obtained as the residue was purified by column chromatography with a column containing 45 g of silica gel, the column is eluted with a mixture of petroleum ether and ethyl acetate (4:1) to afford the target compound 1-(5-cyclopropyl-3-nitro-2-pyridyl)-4-methyl-piperidin-4-ol.

Step-3. To a solution of 1-(5-cyclopropyl-3-nitro-2-pyridyl)-4-methyl-piperidin-4-ol (600 mg, 2.16 mmol) in MeOH (20 mL) was added Palladium (60 mg, 563.80 μmol) under H₂. The mixture was stirred at 25° C. for 2 hr. TLC analysis showed target product formed. The reaction mixture was filtered and concentrated under reduced pressure to give 1-(3-amino-5-cyclopropyl-2-pyridyl)-4-methyl-piperidin-4-ol.

Step-4. To a solution of 1-(3-amino-5-cyclopropyl-2-pyridyl)-4-methyl-piperidin-4-ol (250 mg, 1.01 mmol) and 5-bromofuran-2-carboxylic acid (193.04 mg, 1.01 mmol) in dichloromethane (5 mL), added pyridine (1.28 g, 16.14 mmol, 1.3 mL) and phosphoryl trichloride (154.98 mg, 1.01 mmol, 94.22 μL) at 0° C. The reaction mixture was stirred at 25° C. for 30 mins, LCMS analysis showed target product formed. Water (15 mL) was added, the combined aqueous layers are extracted with ethyl acetate (3×15 mL) and the combined organic layers are dried with brine (2×15 mL), sodium sulfate (150 g) and concentrated in vacuum. The crude material obtained as the residue was purified by column chromatography with a column containing 10 g of silica gel, the column is eluted with a mixture of petroleum ether and ethyl acetate (10:1) to afford the target compound 5-bromo-N-[5-cyclopropyl-2-(4-hydroxy-4-methyl-1-piperidyl)-3-pyridyl]furan-2-carboxamide.

Step-5. To a solution of 5-bromo-N-[5-cyclopropyl-2-(4-hydroxy-4-methyl-1-piperidyl)-3-pyridyl]furan-2-carboxamide (50 mg, 118.96 μmol), sodium carbonate (63.04 mg, 594.81 μmol, 24.90 μL), [1,1′-Bis(diphenylphosphino) ferrocene]dichloropalladium (II) (8.70 mg, 11.90 μmol) and 1H-pyrazol-4-ylboronic acid (13.31 mg, 118.96 μmol) in 1,4-dioxane (4 mL) and water (1 mL). After deoxygenating the flask with three alternating vacuum and purge cycles, the reaction mixture was stirred at 100° C. for 16 hrs. LCMS analysis showed completed consumption of 5-bromo-N-[5-cyclopropyl-2-(4-hydroxy-4-methyl-1-piperidyl)-3-pyridyl]furan-2-carboxamide, the target product was formed. The mixture was filtered through a 6-cm fritted glass funnel, added water (10 mL) in the filtrate. The combined aqueous layers are extracted with ethyl acetate (3×10 mL) and the combined organic layers washed with water (3×10 mL), dried with brine (2×10 mL), sodium sulfate (150 g) and concentrated in vacuum. The crude material obtained as the residue was purified by column chromatography with a column containing 5 g of silica gel, the column is eluted with a mixture of petroleum ether and ethyl acetate (1:1) to afford the target compound N-[5-cyclopropyl-2-(4-hydroxy-4-methyl-1-piperidyl)-3-pyridyl]-5-(1H-pyrazol-4-yl) furan-2-carboxamide.

¹H NMR (400 MHz, DMSO-d₆) δ 9.19 (s, 1H), 8.08 (d, J=2.2 Hz, 3H), 7.92 (d, J=2.3 Hz, 1H), 7.32 (d, J=3.6 Hz, 1H), 6.77 (d, J=3.6 Hz, 1H), 4.32 (s, 1H), 3.10 (t, J=12.7 Hz, 2H), 2.94-2.89 (m, 2H), 1.94 (d, J=13.6 Hz, 1H), 1.76-1.62 (m, 4H), 1.19 (s, 3H), 0.97 (q, J=6.3 Hz, 2H), 0.68-0.64 (m, 2H).

LCMS (ESI) m/z: [M+H]⁺ 408.33; Purity=100% (254 nm); Retention time=2.29 min.

### Example 11: Preparation of N-[2-(4-hydroxy-1-piperidyl)-5-(trifluoromethyl)-3-pyridyl]-5-(1H-pyrazol-4-yl)pyridine-2-carboxamide (Compound 097)

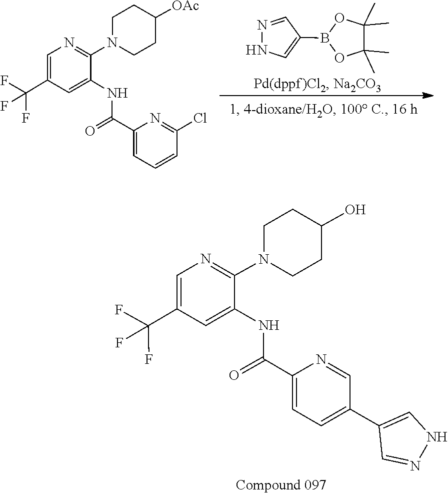 [SMILES: CC(=O)OC1CCN(c2ncc(C(F)(F)F)cc2NC(=O)c2cccc(Cl)n2)CC1 ; CC1(C)OB(c2cn[nH]c2)OC1(C)C ; O=C(Nc1cc(C(F)(F)F)cnc1N1CCC(O)CC1)c1ccc(-c2cn[nH]c2)cn1]

A solution of [1-[3-[(6-chloropyridine-2-carbonyl)amino]-5-(trifluoromethyl)-2-pyridyl]-4-piperidyl]acetate (100 mg, 0.23 mmol), 4-(4,4,5,5-tetramethyl-1,3,2-dioxaborolan-2-yl)-1H-pyrazole (131 mg, 0.68 mmol), sodium carbonate (120 mg, 1.13 mmol) and [1,1′-Bis(diphenylphosphino) ferrocene]dichloropalladium (II) (17 mg, 0.023 mmol) in 1,4-dioxane (2 atmosphere. LCMS analysis showed target product formed. The mixture was quenched with water (30 mL). The combined aqueous layers are extracted with ethyl acetate (3×20 mL), and the combined organic layers are dried with brine (2×20 mL), sodium sulfate (10 g) and concentrated in vacuum. The crude product was purified by prep-HPLC to afford the target compound N-[2-(4-hydroxy-1-piperidyl)-5-(trifluoromethyl)-3-pyridyl]-5-(1H-pyrazol-4-yl)pyridine-2-carboxamide.

¹HNMR (400 MHz, DMSO-_{d6}) δ 13.24 (s, 1H), 10.29 (s, 1H), 9.06 (d, J=1.2 Hz, 1H), 8.88 (d, J=2.4 Hz, 1H), 8.45 (d, J=3.2 Hz, 1H), 8.33 (d, J=1.6 Hz, 1H), 8.31 (d, J=4.4 Hz, 2H), 8.18-8.16 (m, 2H), 4.81 (d, J=2.8 Hz, 1H), 3.79-3.73 (m, 1H), 3.46-3.41 (m, 2H), 3.01-2.95 (m, 2H), 1.99-1.94 (m, 2H), 1.75-1.67 (m, 2H).

LCMS (ESI) m/z: [M+H]⁺ 433.61; Purity=100% (254 nm); Retention time=2.87 min.

### Example 12: Preparation of N-(2-(2-oxo-7-azaspiro[3.5]nonan-7-yl)-5-(trifluoromethyl)pyridin-3-yl)-5-(tetrahydro-2H-pyran-4-yl) furan-2-carboxamide (Compound 065)

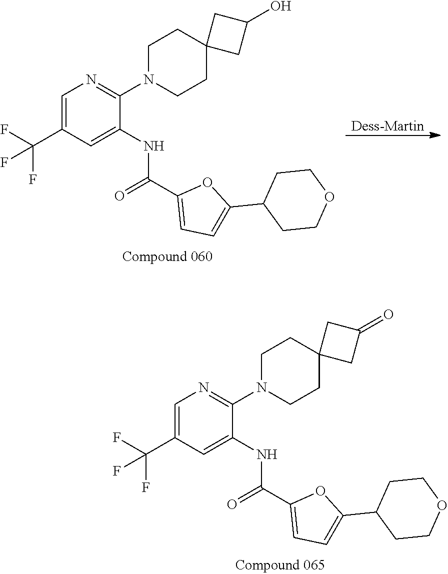 [SMILES: O=C(Nc1cc(C(F)(F)F)cnc1N1CCC2(CC1)CC(O)C2)c1ccc(C2CCOCC2)o1 ; O=C1CC2(CCN(c3ncc(C(F)(F)F)cc3NC(=O)c3ccc(C4CCOCC4)o3)CC2)C1]

Step-1. To a solution of N-[2-(2-hydroxy-7-azaspiro[3.5]nonan-7-yl)-5-(trifluoromethyl)-3-pyridyl]-5-tetrahydropyran-4-yl-furan-2-carboxamide (12 mg, 0.03 mmol) in dichloromethane (3 mL). To that solution was added (1,1-diacetoxy-3-oxo-1,2-benziodoxol-1-yl)acetate (21 mg, 0.05 mmol), the reaction mixture was stirred at 25° C. for 16 hours. TLC analysis indicated the total consumption of the starting material. The mixture was filtered through a 6-cm fritted glass funnel, added water (15 mL) in the filtrate. The combined aqueous layers are extracted with ethyl acetate (3×15 mL) and the combined organic layers washed with water (3×10 mL), dried with brine (2×50 mL), sodium sulfate (15 g) and concentrated in vacuum. The crude material obtained as the residue was purified by prep-TLC (dichloromethane/methanol=15:1) to afford the target compound N-[2-(2-oxo-7-azaspiro[3.5]nonan-7-yl)-5-(trifluoromethyl)-3-pyridyl]-5-tetrahydropyran-4-yl-furan-2-carboxamide.

LCMS (ESI) m/z: [M+H]⁺ 478.27; Purity=100.00% (254 nm); Retention time=3.10 min.

¹H NMR (400 MHz, DMSO-d6) δ 9.46 (s, 1H), 8.47-8.42 (m, 1H), 8.36 (d, J=2.4 Hz, 1H), 7.29 (d, J=4.0 Hz, 1H), 6.42 (d, J=4.0 Hz, 1H), 3.96-3.86 (m, 2H), 3.48-3.41 (m, 2H), 3.27-3.24 (m, 4H), 3.09-2.97 (m, 1H), 2.87 (s, 4H), 1.94-1.62 (m, 8H).

### Example 13: Preparation of N-[2-(4-hydroxy-1-piperidyl)-5-(trifluoromethyl)-3-pyridyl]-5-(1-methyl-4-piperidyl) furan-2-carboxamide (Compound 066)

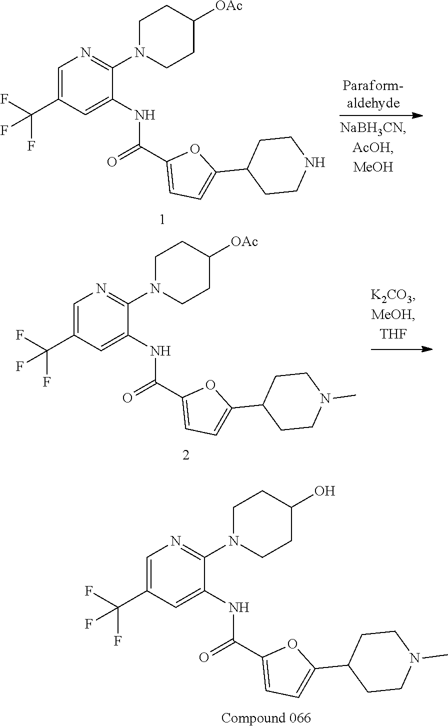 [SMILES: CC(=O)OC1CCN(c2ncc(C(F)(F)F)cc2NC(=O)c2ccc(C3CCNCC3)o2)CC1 ; CC(=O)OC1CCN(c2ncc(C(F)(F)F)cc2NC(=O)c2ccc(C3CCN(C)CC3)o2)CC1 ; CN1CCC(c2ccc(C(=O)Nc3cc(C(F)(F)F)cnc3N3CCC(O)CC3)o2)CC1]

Step-1. To a solution of [1-[3-[5-(4-piperidyl) furan-2-carbonyl]amino]-5-(trifluoromethyl)-2-pyridyl]-4-piperidyl]acetate (40 mg, 0.083 mmol) in MeOH (8 mL) was added paraformaldehyde (22 mg, 0.25 mmol) and a drop of acetic acid, the reaction solution was stirred at 25° C. for 1 h, then sodium cyanoborohydride (26 mg, 0.42 mmol) was added, the mixture solution was stirred at 25° C. for 4 h, TLC showed the starting material was consumed up and the product formed, water was added to quench the reaction and extracted with EA, the combined organic layer was dried over anhydrous Na₂SO₄, filtered and concentrated to give a residue. The residue was purified by prep-TLC (DCM:MeOH=9:1) to give [1-[3-[5-(1-methyl-4-piperidyl) furan-2-carbonyl]amino]-5-(trifluoromethyl)-2-pyridyl]-4-piperidyl]acetate.

Step-2. a solution of [1-[3-[[5-(1-methyl-4-piperidyl) furan-2-carbonyl]amino]-5-(trifluoromethyl)-2-pyridyl]-4-piperidyl]acetate (20 mg, 0.04 mmol) in THF (3 mL) was added K₂CO₃ (17 mg, 0.012 mmol) and MeOH (3 mL), and the mixture solution was stirred at 25° C. for 4 h. Then the mixture solution was filtered and the filtrate was concentrated to give the residue, the residue was purified by prep-TLC (DCM:MeOH=9:1) to N-[2-(4-hydroxy-1-piperidyl)-5-(trifluoromethyl)-3-pyridyl]-5-(1-methyl-4-piperidyl) furan-2-carboxamide.

¹H NMR (400 MHz, DMSO-d₆) δ 9.43 (s, 1H), 8.42 (s, 1H), 8.28 (d, J=2.0 Hz, 1H), 7.27 (d, J=3.6 Hz, 1H), 6.40 (d, J=4.0 Hz, 1H), 4.76 (s, 1H), 3.73-3.66 (m, 1H), 3.61-3.56 (m, 2H), 3.04-2.98 (m, 2H), 2.83-2.81 (m, 2H), 2.73-2.65 (m, 1H), 2.19 (s, 3H), 2.04-1.92 (m, 4H), 1.88-1.84 (m, 2H), 1.73-1.63 (m, 2H), 1.59-1.50 (m, 2H).

LCMS (ESI) m/z: [M+H⁺ 453.3; Purity=100% (254 nm); Retention time=2.46 min.

### Example 14: Preparation of N-(2-(1-methylpiperidin-4-yl)-5-(trifluoromethyl)pyridin-3-yl)-5-(tetrahydro-2H-pyran-4-yl) furan-2-carboxamide (Compound 071)

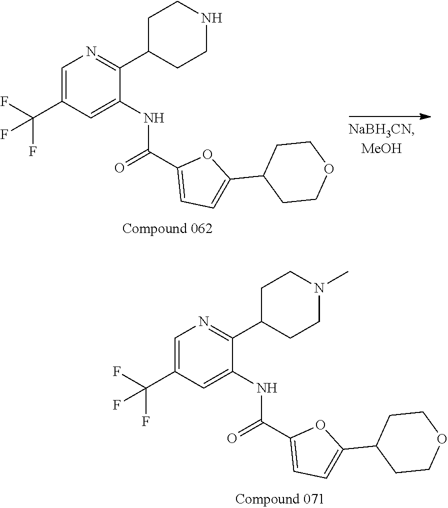 [SMILES: O=C(Nc1cc(C(F)(F)F)cnc1C1CCNCC1)c1ccc(C2CCOCC2)o1 ; CN1CCC(c2ncc(C(F)(F)F)cc2NC(=O)c2ccc(C3CCOCC3)o2)CC1]

Step-1. A solution of N-(2-(piperidin-4-yl)-5-(trifluoromethyl)pyridin-3-yl)-5-(tetrahydro-2H-pyran-4-yl) furan-2-carboxamide (20 mg, 0.05 mmol), Paraformaldehyde (40 mg) in MeOH (5 mL) was added Sodium cyanoborohydride (10 mg, 0.15 mmol) stirred at room temperature for 4 hours. LCMS analysis showed target product formed. The mixture was quenched with water (25 mL). The combined aqueous layers are extracted with ethyl acetate (3×20 mL), and the combined organic layers are dried with brine (2×25 mL), sodium sulfate (25 g) and concentrated in vacuum. The crude product was purified by prep-TLC (dichloromethane/methanol=15:1) to afford the target compound N-(2-(1-methylpiperidin-4-yl)-5-(trifluoromethyl)pyridin-3-yl)-5-(tetrahydro-2H-pyran-4-yl) furan-2-carboxamide.

LCMS (ESI) m/z: [M+H]⁺ 438.25; Purity=100% (254 nm); Retention time=2.11 min.

¹HNMR (400 MHz, DMSO-d6) δ 10.06 (s, 1H), 8.82 (s, 1H), 8.16 (d, J=1.6 Hz, 1H), 7.27 (d, J=3.2 Hz, 1H), 6.41 (d, J=3.2 Hz, 1H), 3.98-3.89 (m, 2H), 3.53-3.41 (m, 2H), 3.10-2.98 (m, 2H), 2.92-2.81 (m, 2H), 2.17 (s, 3H), 1.99-1.77 (m, 6H), 1.76-1.62 (m, 4H).

### Example 15: Preparation of N-[2-(4-hydroxy-1-piperidyl)-5-(trifluoromethyl)-3-pyridyl]pyridine-4-carboxamide (Compound 046)

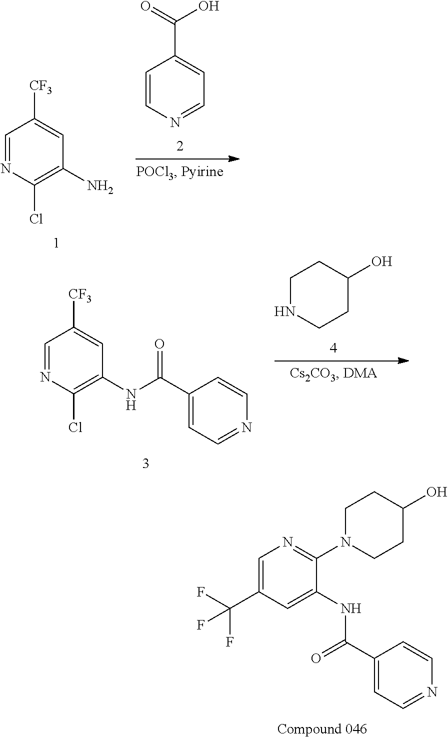 [SMILES: Nc1cc(C(F)(F)F)cnc1Cl ; O=C(O)c1ccncc1 ; O=C(Nc1cc(C(F)(F)F)cnc1Cl)c1ccncc1 ; OC1CCNCC1 ; O=C(Nc1cc(C(F)(F)F)cnc1N1CCC(O)CC1)c1ccncc1]

Step 1. A solution of 2-chloro-5-(trifluoromethyl)pyridin-3-amine (100 mg, 5.09 mmol) isonicotinic acid (63 mg, 5.09 mmol) and Pyridine (1.18 g, 14.90 mmol, 1.2 mL) in Dichloromethane (3 mL) was stirred at 0° C. for 5 minutes under nitrogen atmosphere. Then, POCl₃ (987 mg, 6.44 mmol, 0.6 mL) was added at 0° C. and the reaction mixture was stirred at 25° C. for 30 minutes. LCMS analysis showed target product formed and the mixture was quenched with water (25 mL). The combined aqueous layers were extracted with ethyl acetate (3×20 mL), dried with brine (2×25 mL) and sodium sulfate (25 g), and concentrated in vacuum. The crude product was purified by prep-TLC (petroleum ether/ethyl acetate=1:1) to afford the target compound N-[2-chloro-5-(trifluoromethyl)-3-pyridyl]pyridine-4-carboxamide.

Step 2. A solution of N-[2-chloro-5-(trifluoromethyl)-3-pyridyl]pyridine-4-carboxamide (74 mg, 2.45 mmol) piperidin-4-ol (25 mg, 2.45 mmol) and Cesium carbonate (240 mg, 7.36 mmol) in DMA (3 mL) was stirred at 100° C. for 18 hours. TLC analysis indicated the total consumption of the starting material. The mixture was filtered through a 6-cm fritted glass funnel, added water (10 mL) in the filtrate. The combined aqueous layers are extracted with ethyl acetate (3×10 mL) and washed with water (3×10 mL), dried with brine (2×20 mL), sodium sulfate (10 g) and concentrated in vacuum. The crude product was purified by prep-TLC (Petroleum ether/Ethyl acetate=1:1) to afford the target compound N-[2-(4-hydroxy-1-piperidyl)-5-(trifluoromethyl)-3-pyridyl]pyridine-4-carboxamide.

LCMS (ESI) m/z: [M+H]⁺ 367.0; Purity=97% (254 nm); Retention time=1.19 min

¹HNMR (400 MHz, DMSO-d6) δ 10.27 (s, 1H), 8.81 (d, J=8.0 Hz, 2H), 8.44 (s, 1H), 8.06 (s, 1H), 7.87 (d, J=4.0 Hz, 2H), 4.71 (d, J=4.0 Hz, 1H), 3.77-3.63 (m, 3H), 3.09-3.04 (m, 2H), 1.80-1.75 (m, 2H), 1.49-1.40 (m, 2H).

### Example 16. Preparation of 5-(3-amino-1H-pyrazol-4-yl)-N-[2-(4-hydroxy-1-piperidyl)-5-(trifluoromethyl)-3-pyridyl]furan-2-carboxamide (Compound 106)

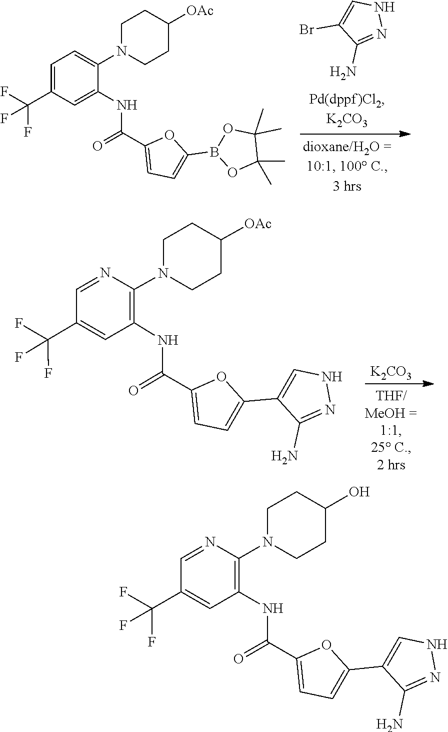

### Experimental Procedure

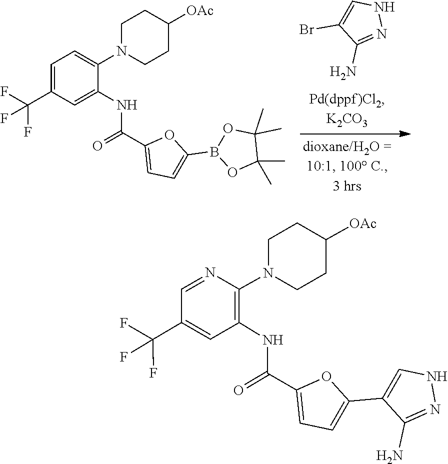 [SMILES: CC(=O)OC1CCN(c2ccc(C(F)(F)F)cc2NC(=O)c2ccc(B3OC(C)(C)C(C)(C)O3)o2)CC1 ; Nc1n[nH]cc1Br ; CC(=O)OC1CCN(c2ncc(C(F)(F)F)cc2NC(=O)c2ccc(-c3c[nH]nc3N)o2)CC1]

Step 1. To a solution of [1-[2-[[5-(4,4,5,5-tetramethyl-1,3,2-dioxaborolan-2-yl) furan-2-carbonyl]amino]-4-(trifluoromethyl)phenyl]-4-piperidyl]acetate (100 mg, 191.45 μmol) and 4-bromo-1H-pyrazol-3-amine (31.01 mg, 191.45 μmol) in 1,4-dioxane (2 mL) and water (0.2 mL), added [2-(2-aminoethyl)phenyl]-chloro-palladium; dicyclohexyl-[2-(2,6-dimethoxyphenyl)phenyl]phosphane (12.88 mg, 19.15 μmol). The reaction mixture was stirred at 100° C. for 3 hours. LCMS showed the target product formed. The reaction mixture was extracted with ethyl acetate (3×100 mL), and the combined organic layers washed with water (100 mL), dried with brine (100 mL), dried over sodium sulfate (50 g), and concentrated in vacuum. The residue was purified by prep-HPLC to afford the target compound [1-[3-[5-(3-amino-1H-pyrazol-4-yl) furan-2-carbonyl]amino]-5-(trifluoromethyl)-2-pyridyl]-4-piperidyl]acetate.

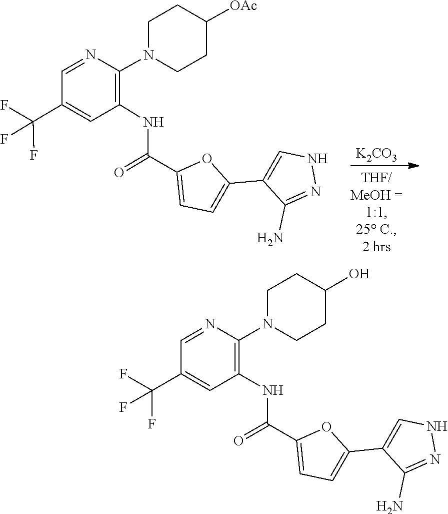

To a solution of [1-[3-[[5-(3-amino-1H-pyrazol-4-yl) furan-2-carbonyl]amino]-5-(trifluoromethyl)-2-pyridyl]-4-piperidyl]acetate (5 mg, 10.45 μmol) in tetrahydrofuran (1 mL) and methanol (1 mL), Then added potassium carbonate (4.33 mg, 31.35 μmol) the reaction mixture was stirred at 25° C. for 2 hours. LCMS analysis indicated the target consumption was formed. The mixture was purified by prep-HPLC to afford the target product 5-(3-amino-1H-pyrazol-4-yl)-N-[2-(4-hydroxy-1-piperidyl)-5-(trifluoromethyl)-3-pyridyl]furan-2-carboxamide.

1H NMR (400 MHz, DMSO-_{d6}) δ 8.41 (m, 1H), 8.19 (d, J=2.4 Hz, 1H), 7.77 (s, 1H), 7.33 (d, J=4 Hz, 1H), 6.61 (d, J=4.0 Hz, 1H), 5.36-5.29 (m, 1H), 3.69-3.63 (m, 3H), 3.08-3.01 (m, 2H), 2.05-1.93 (m, 2H), 1.88-1.76 (m, 2H).

LCMS (ESI) m/z: [M+H]⁺ 437.13; Purity=97.65% (254 nm); Retention time=1.36 min.

The following compounds were prepared in a similar manner to the procedure described above.

| | LCMS | |
|---|---|---|
| Compound | (ESI): | |
| No. | [M + H]⁺ | ¹H NMR |
| 108 | 423.20 | ¹H NMR (400 MHz, DMSO-d6) δ 8.43 (d, J = 1.2 Hz, 1H), 8.28- |
| | | 8.19 (m, 2H), 7.45 (d, J = 3.6 Hz, 1H), 6.97 (d, J = 3.5 Hz, 1H), |
| | | 3.76-3.62 (m, 3H), 3.10-3.00 (m, 2H), 1.90-1.82 (m, 2H), |
| | | 1.64-1.50 (m, 2H) |
| 110 | 436.2 | ¹H NMR (400 MHz, DMSO-d6) δ 13.34 (s, 1H), 9.62 (s, 1H), |
| | | 8.85 (d, J = 2.2 Hz, 1H), 8.55 (s, 1H), 8.24 (s, 1H), 7.97 (s, 1H), |
| | | 7.41 (d, J = 3.6 Hz, 1H), 6.85 (d, J = 3.6 Hz, 1H), 4.93 (d, J = |
| | | 3.4 Hz, 1H), 3.71 (s, 1H), 3.42 (s, 1H), 3.07 (d, J = 12.4 Hz, 1H), |
| | | 2.75 (t, J = 11.9 Hz, 1H), 2.06 (d, J = 12.5 Hz, 1H), 1.97 (d, J = |
| | | 11.5 Hz, 1H), 1.56 (s, 1H), 1.49-1.39 (m, 1H), 0.92 (d, J = 6.1 |
| | | Hz, 3H) |
| 111 | 462.3 | ¹H NMR (400 MHz, DMSO-d6) δ 9.96 (s, 1H), 8.90 (d, J = 2.3 |
| | | Hz, 1H), 8.60-8.56 (m, 1H), 8.03 (d, J = 5.3 Hz, 1H), 7.48 (d, |
| | | J = 3.7 Hz, 1H), 7.35 (d, J = 3.7 Hz, 1H), 7.00 (dd, J = 5.3, 1.4 |
| | | Hz, 1H), 6.88 (s, 1H), 6.20 (s, 2H), 5.42 (d, J = 3.1 Hz, 1H), 3.78 |
| | | (s, 1H), 3.37 (s, 1H), 3.07 (d, J = 12.2 Hz, 1H), 2.79 (t, J = 13.1 |
| | | Hz, 1H), 2.12 (d, J = 12.6 Hz, 1H), 2.03 (d, J = 10.1 Hz, 1H), |
| | | 1.73 (q, J = 11.5, 10.9 Hz, 1H), 1.48 (q, J = 10.7 Hz, 1H), 0.88 |
| | | (d, J = 6.1 Hz, 3H). |
| 112 | 424.2 | ¹H NMR (400 MHz, DMSO-d6) δ 9.57 (s, 1H), 8.42 (d, J = 1.2 |
| | | Hz, 1H), 8.19 (d, J = 2.4 Hz, 1H), 7.29 (d, J = 3.2 Hz, 1H), 6.51 |
| | | (d, J = 3.6 Hz, 2H), 4.74 (d, J = 2.8 Hz, 1H), 4.40-4.36 (m, 2H), |
| | | 3.69-3.52 (m, 4H), 3.05-3.00 (m, 2H), 2.95-2.89 (m, 1H), |
| | | 2.79-2.73 (m, 1H), 2.27-2.22 (m, 1H), 2.04-2.00 (m, 1H), |
| | | 1.84-1.82 (m, 2H), 1.55-1.48 (m, 2H). |
| 113 | 436.2 | ¹H NMR (400 MHz, DMSO-d6) δ 13.25 (s, 1H), 9.57 (s, 1H), |
| | | 8.47 (s, 1H), 8.38 (d, J = 2.0 Hz, 1H), 8.24 (s, 1H), 7.95 (s, 1H), |
| | | 7.38 (d, J = 3.6 Hz, 1H), 6.79 (d, J = 3.6 Hz, 1H), 4.68 (d, J = 4.0 |
| | | Hz, 1H), 4.11-4.07 (m, 1H), 3.95-3.89 (m, 1H), 3.56-3.51 |
| | | (m, 1H), 3.10-3.04 (m, 1H), 1.87-1.83 (m, 1H), 1.75-1.70 |
| | | (m, 1H), 1.67-1.57 (m, 2H), 1.08 (d, J = 6.8 Hz, 3H). |
| 114 | 462.2 | ¹H NMR (400 MHz, DMSO-d6) δ 9.81 (s, 1H), 8.48 (s, 1H), |
| | | 8.30 (d, J = 2.0 Hz, 1H), 8.02 (d, J = 5.2 Hz, 1H), 7.47 (d, J = 3.6 |
| | | Hz, 1H), 7.27 (d, J = 3.6 Hz, 1H), 6.99 (d, J = 6.4 Hz, 1H), 6.87 (s, |
| | | 1H), 6.10 (s, 2H), 4.78 (d, J = 4.0 Hz, 1H), 4.17-4.14 (m, 1H), |
| | | 3.94-3.89 (m, 1H), 3.61-3.57 (m, 1H), 3.15-3.09 (m, 1H), |
| | | 1.88-1.85 (m, 1H), 1.76-1.73 (m, 1H), 1.66-1.53 (m, 2H), |
| | | 1.10 (d, J = 6.4 Hz, 3H). |
| 115 | 456.1 | ¹H NMR (400 MHz, DMSO-d6) δ 12.48 (s, 1H), 10.00 (s, 1H), |
| | | 8.45 (d, J = 0.8 Hz, 1H), 8.06 (d, J = 1.6 Hz, 1H), 7.42 (d, J = |
| | | 3.6 Hz, 2H), 6.87 (d, J = 3.6 Hz, 1H), 4.43 (s, 2H), 3.61 (t, J = |
| | | 5.6 Hz, 2H), 2.79 (t, J = 5.2 Hz, 2H). |
| 118 | 409.25 | ¹H NMR (400 MHz, DMSO-d6) δ 13.31 (s, 1H), 9.59 (s, 1H), |
| | | 8.20 (d, J = 2 Hz, 1H), 7.98 (d, J = 2.4 Hz, 2H), 7.32 (d, J = 3.6 |
| | | Hz, 1H), 6.80 (d, J = 3.2 Hz, 1H), 4.66 (d, J = 2.8 Hz, 1H), |
| | | 4.02-3.98 (m, 1H), 3.63-3.58 (m, 1H), 3.15-3.09 (m, 1H), 2.75 |
| | | (m, 1H), 1.95 (m, 1H), 1.86-1.80 (m, 2H), 1.62 (m, 2H), |
| | | 1.02-0.98 (m, 2H), 0.80 (d, J = 6.4 Hz, 3H), 0.68 (m 2H). |
| 119 | 434.21 | ¹H NMR (400 MHz, DMSO-d6) δ 9.67 (s, 1H), 8.16 (s, 1H), |
| | | 8.00 (s, 2H), 7.40 (s, 1H), 7.27 (s, 1H), 6.94 (s, 1H), 6.82 (s, |
| | | 1H), 6.12 (s, 2H), 4.79 (s, 1H), 4.00 (s, 1H), 2.87 (s, 1H), 1.97 |
| | | (s, 1H), 1.87 (s, 2H), 1.69 (s, 2H), 1.00 (s, 2H), 0.84 (s, 3H), |
| | | 0.68 (s, 2H). |
| 121 | 416.30 | ¹H NMR (400 MHz, DMSO) δ 13.18 (s, 1H), 12.41 (s, 1H), 9.40 |
| | | (s, 1H), 8.05-7.73 (m, 4H), 7.47-7.29 (m, 2H), 6.74 (d, J = |
| | | 3.5 Hz, 1H), 4.16 (s, 2H), 3.30 (s, 2H), 2.89-2.79 (m, 2H), |
| | | 2.01-1.87 (m, 1H), 1.01-0.92 (m, 2H), 0.71-0.63 (m, 2H). |
| 122 | 442.30 | ¹H NMR (400 MHz, DMSO-d6) δ 12.54 (s, 1H), 9.62 (s, 1H), |
| | | 8.05-7.86 (m, 3H), 7.55-7.19 (m, 3H), 6.78-6.58 (m, 2H), |
| | | 6.11 (s, 2H), 4.14 (s, 2H), 3.34 (s, 2H), 2.87 (s, 2H), 2.00-1.87 |
| | | (m, 1H), 1.03-0.92 (m, 2H), 0.71-0.60 (m, 2H). |
| 123 | 444.26 | ¹H NMR (400 MHz, DMSO-d6) δ 8.46 (s, 1H), 8.16 (s, 1H), |
| | | 8.06 (s, 2H), 7.44 (s, 1H), 7.39 (d, J = 4.0 Hz, 1H), 6.76 (d, J = |
| | | 4.0 Hz, 1H), 4.45 (s, 2H), 3.63 (t, J = 4.8 Hz, 2H), 2.83 (t, J = |
| | | 4.8 Hz, 2H). |
| 124 | 470.28 | ¹H NMR (400 MHz,) δ 12.47 (s, 1H), 10.04 (s, 1H), 8.47 (s, |
| | | 1H), 8.17 (s, 1H), 7.99 (d, J = 5.2 Hz, 1H), 7.49 (d, J = 3.6 Hz, |
| | | 2H), 7.25 (d, J = 3.6 Hz, 1H), 6.94 (d, J = 5.6 Hz, 1H), 6.81 (s, |
| | | 1H), 6.09 (s, 2H), 4.45 (s, 2H), 3.70-3.59 (m, 2H), 2.89-2.79 |
| | | (m, 2H). |
| 125 | 405.3 | ¹H NMR (400 MHz, DMSO-d6) δ 13.18 (s, 1H), 10.46 (s, 1H), |
| | | 9.04 (d, J = 1.6 Hz, 1H), 8.40-8.33 (m, 3H), 8.30 (dd, J = 8.2, |
| | | 2.2 Hz, 1H), 8.14 (d, J = 8.3 Hz, 1H), 7.92 (d, J = 2.2 Hz, 1H), |
| | | 4.76 (s, 1H), 3.73-3.66 (m, 1H), 3.19-3.13 (m, 2H), 2.81 (t, |
| | | J = 12.3 Hz, 2H), 1.99-1.90 (m, 3H), 1.72 (q, J = 10.9, 9.1 Hz, |
| | | 2H), 1.02-0.95 (m, 2H), 0.70-0.64 (m, 2H). |
| 127 | 408.27 | ¹H NMR (400 MHz, DMSO-d6) δ 13.32 (s, 1H), 9.65 (s, 1H), |
| | | 8.32 (d, J = 2.0 Hz, 1H), 7.99 (d, J = 2.0 Hz, 3H), 7.32 (d, J = |
| | | 3.2 Hz, 1H), 6.82 (d, J = 3.2 Hz, 1H), 4.92 (d, J = 3.2 Hz, 1H), |
| | | 3.64 (m, 2H), 3.29-3.13 (m, 2H), 2.83 (d, J = 11.7 Hz, 1H), |
| | | 2.71 (t, J = 11.6 Hz, 1H), 2.10-1.89 (m, 3H), 1.57 (qd, J = 12.1, |
| | | 4.3 Hz, 1H), 1.36 (q, J = 11.3 Hz, 1H), 1.08-0.96 (m, 2H), 0.77 |
| | | (d, J = 6.0 Hz, 3H), 0.69 (q, J = 5.2 Hz, 2H). |
| 128 | 434.30 | ¹H NMR (400 MHz, DMSO-d6) δ 9.98 (s, 1H), 8.35 (d, J = 2.0 |
| | | Hz, 1H), 8.03 (d, J = 5.2 Hz, 2H), 7.36 (m, 2H), 6.99 (d, J = 5.6 |
| | | Hz, 1H), 6.87 (s, 1H), 6.22 (s, 2H), 5.47 (d, J = 2.8 Hz, 1H), |
| | | 3.82-3.61 (m, 2H), 2.88-2.76 (m, 2H), 2.13-1.96 (m, 3H), |
| | | 1.75-1.65 (m, 1H), 1.43 (q, J = 11.5 Hz, 1H), 1.06-1.00 (m, |
| | | 2H), 0.75 (d, J = 6.4 Hz, 3H), 0.73-0.67 (m, 2H). |
| 129 | 432.3 | ¹H NMR (400 MHz, DMSO-d6) δ 13.08 (s, 1H), 9.88 (s, 1H), |
| | | 8.43-8.39 (m, 1H), 8.20 (s, 2H), 8.11 (d, J = 2.2 Hz, 1H), 7.99 |
| | | (d, J = 8.4 Hz, 2H), 7.79 (d, J = 8.4 Hz, 2H), 4.74 (s, 1H), 3.74 |
| | | (d, J = 13.2 Hz, 2H), 3.66 (s, 1H), 3.04 (t, J = 12.7 Hz, 2H), 1.80 |
| | | (d, J = 9.8 Hz, 2H), 1.48 (q, J = 12.6 Hz, 2H). |
| 130 | 485.3 | ¹H NMR (400 MHz, DMSO-d6) δ 12.52 (s, 1H), 8.44 (s, 1H), |
| | | 8.21 (s, 1H), 7.52-7.41 (m, 2H), 6.99 (d, J = 3.6 Hz, 1H), 6.04 |
| | | (s, 2H), 5.56 (s, 4H), 4.45 (s, 2H), 3.65 (t, J = 5.2 Hz, 2H), 2.86 |
| | | (t, J = 5.2 Hz, 2H). |
| 131 | 457.34 | ¹H NMR (400 MHz, DMSO-d6) δ 12.59 (s, 1H), 9.45 (s, 1H), |
| | | 8.15-7.75 (m, 2H), 7.61-7.31 (m, 2H), 6.98 (d, J = 3.6 Hz, |
| | | 1H), 5.89 (s, 2H), 5.60 (s, 4H), 4.22-4.09 (m, 2H), 3.32 (s, 2H), |
| | | 3.00-2.75 (m, 2H), 1.99-1.89 (m, 1H), 1.03-0.93 (m, 2H), |
| | | 0.73-0.63 (m, 2H). |
| 132 | 477.3 | ¹H NMR (400 MHz, DMSO-d6) δ 9.62 (s, 1H), 8.45 (s, 1H), |
| | | 8.39 (d, J = 2.0 Hz, 1H), 7.42 (d, J = 3.2 Hz, 1H), 7.01 (d, J = 3.6 |
| | | Hz, 1H), 6.10 (s, 2H), 5.56 (s, 4H), 4.85 (d, J-3.6 Hz, 1H), 4.16 |
| | | (d, J = 4.0 Hz, 1H), 3.96-3.90 (m, 1H), 3.56-3.52 (m, 1H), 3.18- |
| | | 3.12 (m, 1H), 1.91-1.88 (m, 1H), 1.81-1.77 (m, 1H), 1.73-1.66 |
| | | (m, 1H), 1.63-1.55 (m, 1H), 1.10 (d, J = 6.8 Hz, 3H). |
| 133 | 477.3 | ¹H NMR (400 MHz, DMSO-d6) δ 13.08 (s, 1H), 9.88 (s, 1H), |
| | | 8.43-8.39 (m, 1H), 8.20 (s, 2H), 8.11 (d, J = 2.2 Hz, 1H), 7.99 |
| | | (d, J = 8.4 Hz, 2H), 7.79 (d, J = 8.4 Hz, 2H), 4.74 (s, 1H), 3.74 |
| | | (d, J = 13.2 Hz, 2H), 3.66 (s, 1H), 3.04 (t, J = 12.7 Hz, 2H), 1.80 |
| | | (d, J = 9.8 Hz, 2H), 1.48 (q, J = 12.6 Hz, 2H). |
| 134 | 449.3 | ¹H NMR (400 MHz, DMSO-d6) δ 9.49 (s, 1H), 8.19 (d, J = 2.3 |
| | | Hz, 1H), 7.98 (d, J = 2.3 Hz, 1H), 7.35 (d, J = 3.7 Hz, 1H), 7.01 |
| | | (d, J = 3.6 Hz, 1H), 6.07 (s, 2H), 5.55 (s, 4H), 4.86 (d, J = 3.1 |
| | | Hz, 1H), 4.00-3.96 (m, 1H), 3.61 (t, J = 5.9 Hz, 1H), 3.10- |
| | | 2.92 (m, 3H), 1.94 (td, J = 8.5, 4.3 Hz, 2H), 1.73-1.62 (m, 2H), |
| | | 1.01-0.97 (m, 2H), 0.87 (d, J = 6.5 Hz, 3H), 0.68 (dq, J = 4.4, |
| | | 3.1, 2.1 Hz, 2H). |
| 135 | 448.52 | ¹H NMR (400 MHz, DMSO-d6) δ 9.98 (s, 1H), 8.36 (d, J = 2.4 |
| | | Hz, 1H), 8.01 (d, J = 2.4 Hz, 1H), 7.34 (d, J = 3.6 Hz, 1H), 7.06 |
| | | (d, J = 3.6 Hz, 1H), 6.12 (s, 2H), 5.67 (s, 4H), 3.75 (m, 1H), |
| | | 3.22-3.14 (m, 1H), 2.91-2.72 (m, 2H), 2.15-1.90 (m, 3H), 1.77- |
| | | 1.67 (m, 1H), 1.44 (m, 1H), 1.09-0.94 (m, 2H), 0.87-0.52 |
| | | (m, 5H). |

### Example 17: Preparation of 5-bromo-N-[2-[(2R,4S)-4-hydroxy-2-methyl-1-piperidyl]-5-(trifluoromethyl)-3-pyridyl]furan-2-carboxamide (Compound 107)

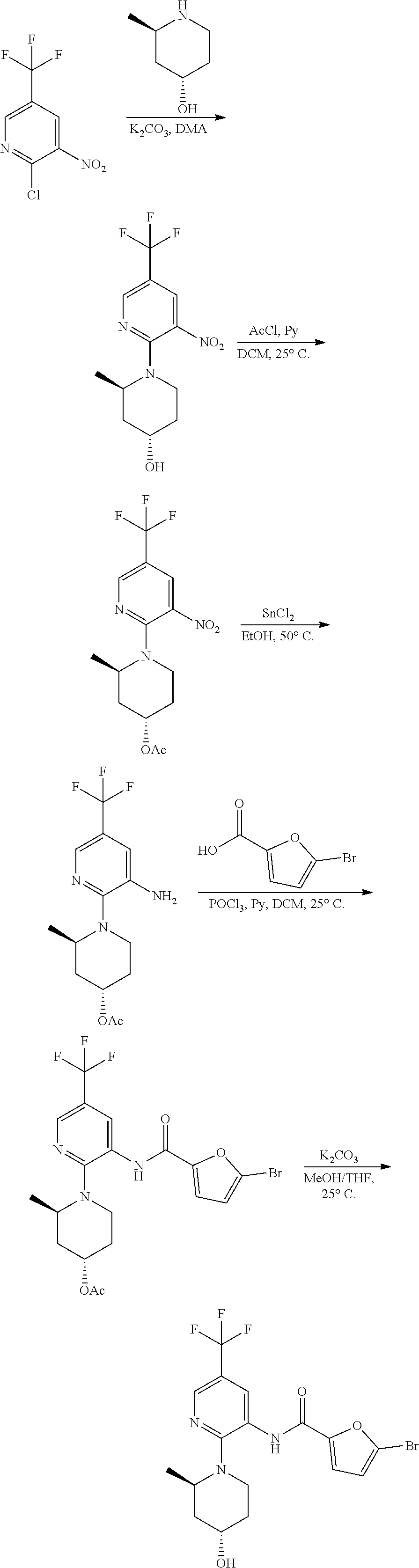

### Experimental Procedure

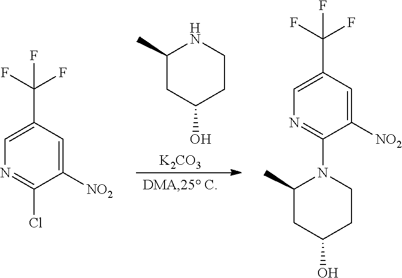

Step 1. To a solution of (2R,4S)-2-methylpiperidin-4-ol (508.40 mg, 4.41 mmol) and 2-chloro-3-nitro-5-(trifluoromethyl)pyridine (1 g, 4.41 mmol) in N,N-dimethylacetamide (15 mL) was added potassium carbonate (1.83 g, 13.24 mmol). The mixture was heated at 25° C. for 18 hours. LCMS analysis showed target product formed. After being cooled, the reaction mixture was extracted with ethyl acetate (100 mL×3) and washed with water (50 mL) and brine (50 mL). The organic layer was dried over anhydrous sodium sulfate, filtered and concentrated under reduced pressure. The crude product was purified by silica column chromatography (petroleum ether:ethyl acetate=3:1) to give (2R,4S)-2-methyl-1-[3-nitro-5-(trifluoromethyl)-2-pyridyl]piperidin-4-ol.

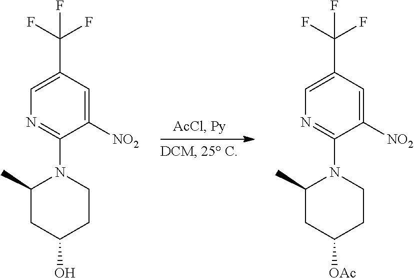 [SMILES: C[C@@H]1C[C@@H](O)CCN1c1ncc(C(F)(F)F)cc1[N+](=O)[O-] ; CC(=O)O[C@H]1CCN(c2ncc(C(F)(F)F)cc2[N+](=O)[O-])[C@H](C)C1]

Step 2. To a solution of (2R,4S)-2-methyl-1-[3-nitro-5-(trifluoromethyl)-2-pyridyl]piperidin-4-ol (1.5 g, 4.91 mmol) in dichloromethane (40 mL) was added pyridine (3.89 g, 49.14 mmol, 3.96 mL) under nitrogen atmosphere. The mixture was stirred at 25° C. for 10 minutes. Then acetyl chloride (3.86 g, 49.14 mmol, 2.98 mL) was added dropwise slowly under nitrogen at 0° C. The mixture was stirred at 25° C. for 4 hours. The reaction mixture was quenched by ice water and extracted with ethyl acetate (150 mL×3) and washed with water (100 mL) and brine (100 mL). The organic layer was dried over anhydrous sodium sulfate, filtered and concentrated under reduced pressure. The crude product was purified by the flash silica gel. chromatography column (petroleum ether:ethyl acetate=5:1) to give [(2R,4S)-2-methyl-1-[3-nitro-5-(trifluoromethyl)-2-pyridyl]-4-piperidyl]acetate.

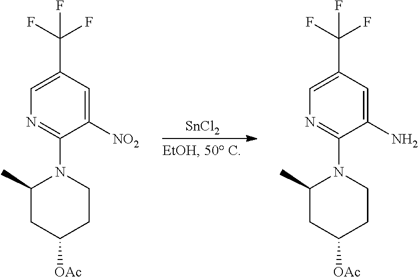 [SMILES: CC(=O)O[C@H]1CCN(c2ncc(C(F)(F)F)cc2[N+](=O)[O-])[C@H](C)C1 ; "SnCl2" ; CC(=O)O[C@H]1CCN(c2ncc(C(F)(F)F)cc2N)[C@H](C)C1]

Step 3. To a solution of [(2R,4S)-2-methyl-1-[3-nitro-5-(trifluoromethyl)-2-pyridyl]-4-piperidyl]acetate (200 mg, 0.6 mmol) in ethanol (5 mL) was added Stannous chloride dihydrate (649.74 mg, 2.88 mmol). The mixture was heated at 50° C. for 1 hour. LCMS analysis showed target product formed. After being cooled, the reaction mixture was acidified with sodium bicarbonate saturated aqueous solution to pH=7, filtered, and diluted with ethyl acetate (100 mL×3) and washed with water (50 mL) and brine (50 mL). The organic layer was dried over anhydrous sodium sulfate, filtered and concentrated under reduced pressure. The crude product was purified by silica column chromatography (petroleum ether:ethyl acetate=4:1) to give [(2R,4S)-1-[3-amino-5-(trifluoromethyl)-2-pyridyl]-2-methyl-4-piperidyl]acetate.

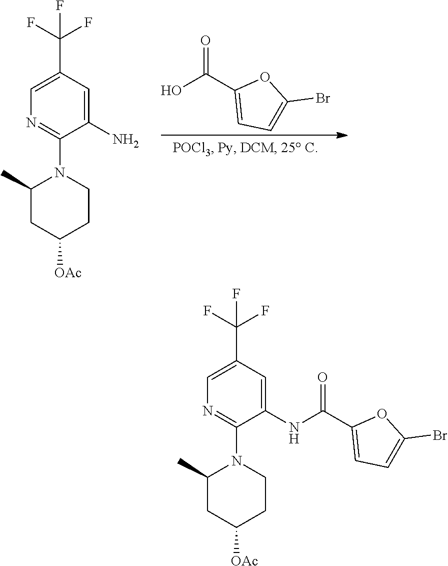 [SMILES: CC(=O)O[C@H]1CCN(c2ncc(C(F)(F)F)cc2N)[C@H](C)C1 ; O=C(O)c1ccc(Br)o1 ; CC(=O)O[C@H]1CCN(c2ncc(C(F)(F)F)cc2NC(=O)c2ccc(Br)o2)[C@H](C)C1]

Step 4. To a solution of 5-bromofuran-2-carboxylic acid (148.66 mg, 0.8 mmol) in dichloromethane (3 mL) was added pyridine (947.28 mg, 11.98 mmol, 1.00 mL) under nitrogen. The mixture was stirred at 25° C. for 10 minutes. Then phosphoryl trichloride (1.84 g, 11.98 mmol, 1.12 mL) was added dropwise slowly under nitrogen at 0° C. The mixture was stirred at 25° C. for 1 hour. The reaction mixture was quenched by water and extracted with ethyl acetate (80 mL×3) and washed with water (50 mL) and brine (50 mL). The organic layer was dried over anhydrous sodium sulfate, filtered, and concentrated under reduced pressure. The crude product was purified by silica column chromatography (petroleum ether:ethyl acetate=4:1) to give [(2R,4S)-1-[3-[(5-bromofuran-2-carbonyl)amino]-5-(trifluoromethyl)-2-pyridyl]-2-methyl-4-piperidyl]acetate.

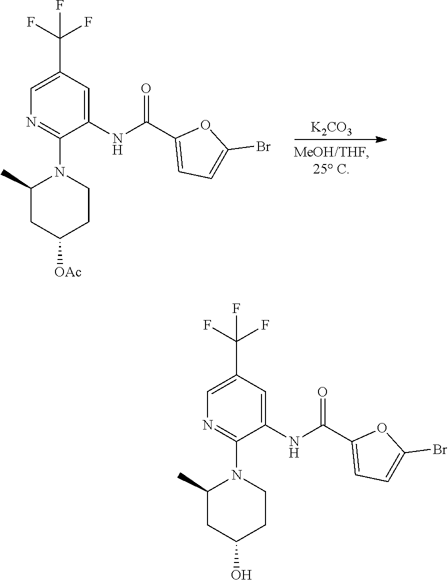

To a solution of [(2R,4S)-1-[3-[(5-bromofuran-2-carbonyl)amino]-5-(trifluoromethyl)-2-pyridyl]-2-methyl-4-piperidyl]acetate (30 mg, 0.06 mmol) in methanol (2 mL) and tetrahydrofuran (2 mL) was added dipotassium carbonate (25.37 mg, 0.1 mmol). The mixture was stirred at 25° C. for 3 hours. LCMS analysis showed target product formed. After being cooled, the reaction mixture was extracted with ethyl acetate (50 mL×3) and washed with water (30 mL) and brine (30 mL). The organic layer was dried over anhydrous sodium sulfate, filtered and concentrated under reduced pressure. The crude product was purified by silica column chromatography (dichloromethane:methanol=10:1) to give 5-bromo-N-[2-[(2R,4S)-4-hydroxy-2-methyl-1-piperidyl]-5-(trifluoromethyl)-3-pyridyl]furan-2-carboxamide.

¹HNMR (400 MHz, DMSO-_{d6}) δ 9.71 (s, 1H), 8.45 (d, J=2.4 Hz, 1H), 8.20 (d, J=2.0 Hz, 1H), 7.38 (d, J=3.6 Hz, 1H), 6.88 (d, J=3.6 Hz, 1H), 4.63 (d, J=4.0 Hz, 1H), 4.17-4.13 (m, 1H), 3.93-3.86 (m, 1H), 3.60-3.55 (m, 1H), 3.09-3.03 (m, 1H), 1.84-1.81 (m, 1H), 1.71-1.67 (m, 1H), 1.58-1.50 (m, 2H), 1.08 (d, J=6.8 Hz, 3H).

LCMS (ESI) m/z: [M+H]⁺ 448.1; Purity=100% (254 nm); Retention time=2.199 min.

The following compounds were prepared in a similar manner to the procedure described above.

| | LCMS | |
|---|---|---|
| Compound | (ESI): | |
| No. | [M + H]⁺ | ¹H NMR |
| 109 | 448.1 | ¹H NMR (400 MHz, DMSO-d6) δ 9.58 (s, 1H), 8.70 (d, J = 2.3 |
| | | Hz, 1H), 8.55 (d, J = 2.2 Hz, 1H), 7.39 (d, J = 3.6 Hz, 1H), 6.92 |
| | | (d, J = 3.6 Hz, 1H), 4.84 (d, J = 3.6 Hz, 1H), 3.72-3.63 (m, |
| | | 1H), 3.50-3.42 (m, 1H), 3.13-3.06 (m, 1H), 2.75 (t, J = 13.0 |
| | | Hz, 1H), 1.94 (dd, J = 12.0, 13.3 Hz, 2H), 1.57 (q, J = 12.8, 12.4 |
| | | Hz, 1H), 1.44-1.35 (m, 1H), 0.92 (d, J = 6.1 Hz, 3H). |
| 115 | 456.1 | ¹H NMR (400 MHz, DMSO-d6) δ 12.48 (s, 1H), 10.00 (s, 1H), |
| | | 8.45 (d, J = 0.8 Hz, 1H), 8.06 (d, J = 1.6 Hz, 1H), 7.42 (d, J = |
| | | 3.6 Hz, 2H), 6.87 (d, J = 3.6 Hz, 1H), 4.43 (s, 2H), 3.61 (t, J = |
| | | 5.6 Hz, 2H), 2.79 (t, J = 5.2 Hz, 2H). |
| 116 | 421.06 | ¹H NMR (400 MHz, DMSO-d6) δ 9.56 (s, 1H), 8.08 (d, J = 2 |
| | | Hz, 1H), 7.98 (d, J = 2.4 Hz, 1H), 7.31 (d, J = 3.6 Hz, 1H), 6.88 |
| | | (d, J = 3.6 Hz, 1H), 4.61 (d, J = 3.2 Hz, 1H), 3.97 (s, 1H), 3.61 |
| | | (m, 1H), 3.11 (m, 1H), 2.79-2.70 (m, 1H), 1.97-1.90 (m, |
| | | 1H), 1.85-1.53 (m, 4H), 0.98 (dt, J = 9.3, 2.9 Hz, 2H), 0.79 |
| | | (d, J = 6.4 Hz, 3H), 0.70-0.64 (m, 2H). |
| 117 | 379.9 | ¹H NMR (400 MHz, DMSO-d6) δ 9.81 (s, 1H), 8.46-8.38 (m, |
| | | 1H), 8.11 (d, J = 2.2 Hz, 1H), 7.39 (d, J = 3.6 Hz, 1H), 7.07 |
| | | (d, J = 3.6 Hz, 1H), 4.93 (s, 1H), 4.73 (s, 1H), 3.73-3.63 (m, |
| | | 3H), 3.08-2.99 (m, 2H), 1.86-1.76 (m, 2H), 1.55-1.42 (m, |
| | | 2H). |
| 120 | 428.2 | ¹H NMR (400 MHz, DMSO-d6) δ 7.95 (d, J = 2.2 Hz, 1H), 7.71 |
| | | (d, J = 2.2 Hz, 1H), 7.44-7.35 (m, 2H), 6.85 (d, J = 3.6 Hz, |
| | | 1H), 6.06 (s, 1H), 4.19 (s, 2H), 3.27 (t, J = 5.6 Hz, 2H), 2.80 |
| | | (t, J = 5.5 Hz, 2H), 1.96-1.87 (m, 1H), 0.98-0.92 (m, 2H), |
| | | 0.68-0.62 (m, 2H). |
| 126 | 420.22 | ¹H NMR (400 MHz, CDCl3) δ 9.68 (s, 1H), 8.35 (d, J = 2.4 Hz, |
| | | 1H), 7.92 (d, J = 2.4 Hz, 1H), 7.10 (d, J = 3.6 Hz, 1H), 6.45 |
| | | (d, J = 3.2 Hz, 1H), 3.79 (m, 1H), 3.26 (m, 1H), 2.83-2.78 (m, |
| | | 2H), 2.13-2.02 (m, 2H), 1.84 (m, 1H), 1.72-1.63 (m, 1H), |
| | | 1.43 (m, 1H), 1.19 (s, 1H), 0.97-0.92 (m, 2H), 0.79 (d, J = 6.4 |
| | | Hz, 3H), 0.70 (m, 2H). |

### Example 18: N-(5-cyclopropyl-2-(4-hydroxy-4-methylpiperidin-1-yl)pyridin-3-yl)-5-(pyridin-4-yl) furan-2-carboxamide (Compound 136)

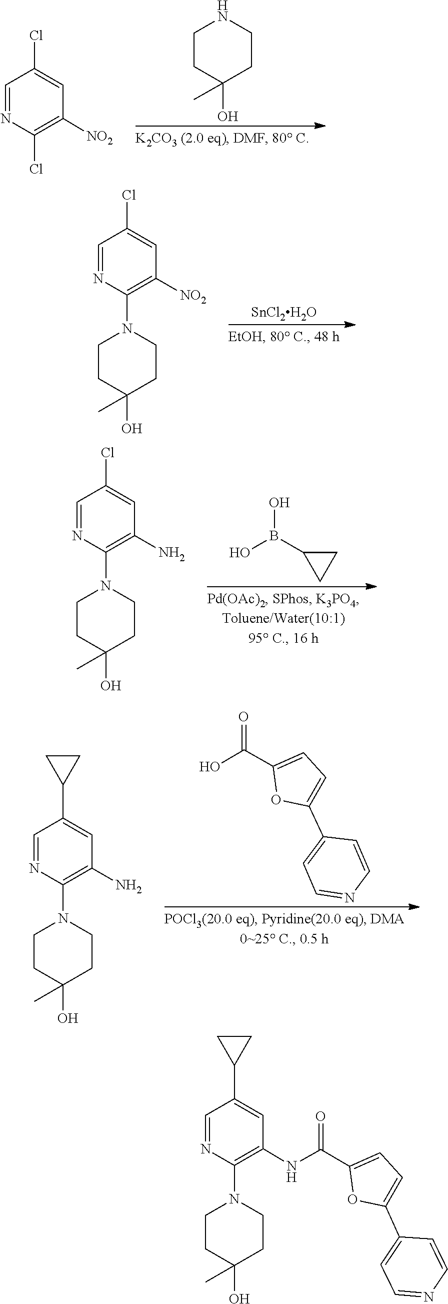

To a solution of 2,5-dichloro-3-nitropyridine (3.00 g, 15.55 mmol) and 4-methylpiperidin-4-ol (1.97 g, 17.10 mmol) in N,N-Dimethylformamide (15 mL) was added potassium carbonate (4.30 g, 31.09 mmol), and the reaction mixture was stirred at 80° C. for 2 hours. The mixture was filtered, water (50 mL) added, and the combined aqueous layers extracted with ethyl acetate (3×50 mL), washed with water (3×50 mL), dried with brine (2×50 mL), sodium sulfate (50 g) and concentrated in vacuum. The crude material was purified by column chromatography to afford the target compound 1-(5-chloro-3-nitropyridin-2-yl)-4-methylpiperidin-4-ol. LCMS (ESI) m/z: [M+H]⁺ 271.9; Purity=98% (254 nm); Retention time=1.41 min.

A solution of 1-(5-chloro-3-nitropyridin-2-yl)-4-methylpiperidin-4-ol (500 mg, 1.84 mmol) and Stannous chloride dihydrate (1.74 g, 9.20 mmol) in ethanol (40 mL) was stirred at 80° C. for 1 hour. Then pH value was adjusted to 7.0 with 100% aqueous sodium bicarbonate solution, and the mixture was filtered, washed with water (20 mL), extracted with ethyl acetate (3×20 mL), washed with water (3×20 mL), dried with brine (2×20 mL), sodium sulfate (25 g) and concentrated in vacuum to afford the target compound 1-(3-amino-5-chloro-2-pyridyl)-4-methyl-piperidin-4-ol. LCMS (ESI) m/z: [M+H]⁺ 242.0; Purity=96% (254 nm); Retention time=1.12 min.

To a solution of 1-(3-amino-5-chloropyridin-2-yl)-4-methylpiperidin-4-ol (2.5 g, 10.34 mmol) and cyclopropylboronic acid (1.33 g, 15.51 mmol) in toluene (30 mL) and water (3 mL) was added potassium phosphate tribasic (5.49 g, 25.86 mmol), Palladium (II) Acetate (232 mg, 1.03 mmol) and 2-dicyclohexylphosphino-2′,6′-dimethoxybiphenyl (425 mg, 1.03 mmol). After deoxygenating the flask, the reaction mixture was stirred at 95° C. for 16 hours. The mixture was filtered, water (100 mL) added, and the layers are extracted with ethyl acetate (3×100 mL). The combined organic layers were washed with water (3×50 mL), dried with brine (2×100 mL), sodium sulfate (150 g) and concentrated in vacuum. The crude material obtained as the residue was purified by column chromatography to afford the target compound 1-(3-amino-5-cyclopropylpyridin-2-yl)-4-methylpiperidin-4-ol. LCMS (ESI) m/z: [M+H]⁺ 248.1; Purity=91% (254 nm); Retention time=0.92 min.

A solution of 1-(3-amino-5-cyclopropylpyridin-2-yl)-4-methylpiperidin-4-ol (1.00 g, 4.04 mmol), 5-(pyridin-4-yl) furan-2-carboxylic acid (918 mg, 4.85 mol) and pyridine (3.26 mL, 40.43 mmol) in N,N-Dimethylacetamide (50 mL) was stirred at 0° C. for 10 minutes. Then, phosphorus oxychloride (3.77 mL, 40.43 mmol) was added. The reaction mixture was stirred at 25° C. for 0.5 h. The mixture was quenched with water (50 mL). The combined aqueous layers are extracted with ethyl acetate (3×50 mL), and the combined organic layers are dried with brine (2×50 mL), sodium sulfate (100 g) and concentrated in vacuum. The crude product was purified by column chromatography to afford the target compound N-(5-cyclopropyl-2-(4-hydroxy-4-methylpiperidin-1-yl)pyridin-3-yl)-5-(pyridin-4-yl) furan-2-carboxamide. LCMS (ESI) m/z: [M+H]⁺ 419.1; Purity=95.74% (254 nm); Retention time=1.53 min. 1HNMR (400 MHz, DMSO-d6) δ 9.50 (s, 1H), 8.68 (dd, J=4.0, 1.6 Hz, 2H), 7.96 (dd, J=8.0, 2.0 Hz, 2H), 7.84 (dd, J=4.0, 1.6 Hz, 2H), 7.51 (d, J=4.0 Hz, 1H), 7.44 (d, J=4.0 Hz, 1H), 4.33 (s, 1H), 3.14-3.08 (m, 2H), 3.01-2.93 (m, 2H), 1.93-1.91 (m, 1H), 1.75-1.60 (m, 4H), 1.18 (s, 3H), 0.97-0.96 (m, 2H), 0.68-0.64 (m, 2H).

### Example 19: N-(5-Cyclopropyl-2-(4-hydroxy-4-methylpiperidin-1-yl)pyridin-3-yl)-5-(tetrahydro-2H-pyran-4-yl) furan-2-carboxamide (Compound 137)

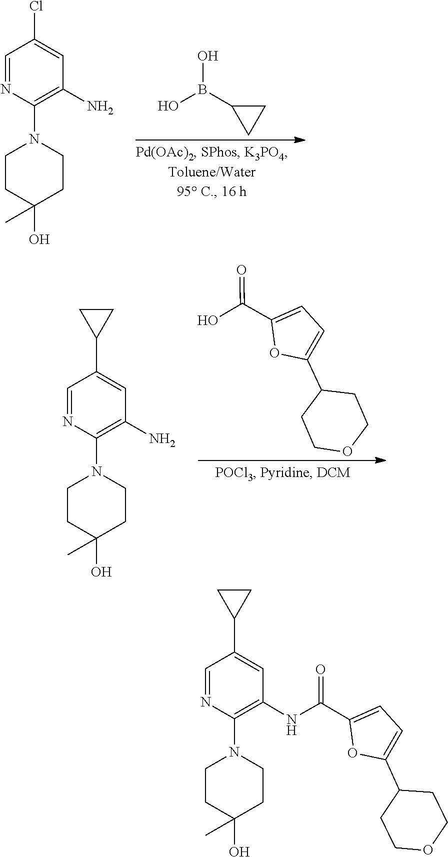 [SMILES: CC1(O)CCN(c2ncc(Cl)cc2N)CC1 ; OB(O)C1CC1 ; CC1(O)CCN(c2ncc(C3CC3)cc2N)CC1 ; O=C(O)c1ccc(C2CCOCC2)o1 ; CC1(O)CCN(c2ncc(C3CC3)cc2NC(=O)c2ccc(C3CCOCC3)o2)CC1]

To a solution of 1-(3-amino-5-chloropyridin-2-yl)-4-methylpiperidin-4-ol (20 mg, 0.08 mmol) and cyclopropylboronic acid (11 mg, 0.12 mmol) in toluene (3 mL) and water (0.5 mL) was added potassium phosphate tribasic (44 mg, 0.21 mmol), palladium (II) acetate (2 mg, 0.08 mmol), and 2-dicyclohexylphosphino-2′,6′-dimethoxybiphenyl (4 mg, 0.08 mmol). After deoxygenating the flask, the reaction mixture was stirred at 95° C. for 16 hours. The mixture was filtered, water (15 mL) added, and the layers extracted with ethyl acetate (3×10 mL), washed with water (3×10 mL), dried with brine (2×10 mL), sodium sulfate (15 g) and concentrated in vacuum. The crude material obtained as the residue was purified by Prep.TLC to afford the target compound 1-(3-amino-5-cyclopropylpyridin-2-yl)-4-methylpiperidin-4-ol. LCMS (ESI) m/z: [M+H]⁺ 248.1; Purity=95% (254 nm); Retention time=0.93 min.

A solution of 1-(3-amino-5-cyclopropylpyridin-2-yl)-4-methylpiperidin-4-ol (10 mg, 0.04 mmol), 5-(tetrahydro-2H-pyran-4-yl) furan-2-carboxylic acid (10 mg, 0.04 mol) in dichloromethane (3 mL) was stirred at 0° C. for 10 minutes. Then, pyridine (64 mg, 0.80 mmol) and phosphorus oxychloride (124 mg, 0.80 mmol) were added at 0° C. The reaction mixture was stirred at 25° C. for 0.5 h. The mixture was quenched with water (25 mL). The combined aqueous layers were extracted with ethyl acetate (3×20 mL), dried with brine (2×25 mL), sodium sulfate (25 g) and concentrated in vacuum. The crude product was purified by prepare TLC to afford the target compound N-(5-cyclopropyl-2-(4-hydroxy-4-methylpiperidin-1-yl)pyridin-3-yl)-5-(tetrahydro-2H-pyran-4-yl) furan-2-carboxamide. LCMS (ESI) m/z: [M+H]⁺ 426.4; Purity=85% (254 nm); Retention time=1.38 min. 1HNMR (400 MHz, DMSO-d6) δ 9.13 (s, 1H), 8.11 (d, J=2.4 Hz, 1H), 7.91 (d, J=2.4 Hz, 1H), 7.21 (d, J=4.0 Hz, 1H), 6.43 (dd, J=4.0, 1.2 Hz, 1H), 4.31 (s, 1H), 3.92 (ddd, J=12.0, 4.0, 1.6 Hz, 2H), 3.44 (td, J=12.0, 2.4 Hz, 2H), 3.09-3.06 (m, 3H), 2.88-2.85 (m, 2H), 1.97-1.87 (m, 3H), 1.76-1.61 (m, 6H), 1.19 (s, 3H), 0.98-0.95 (m, 2H), 0.68-0.61 (m, 2H).

### Biological Assay

The SPRK1 assay was performed as follows:

Method 1: The assay buffer was prepared. The reference compound was diluted to 10 mM. The test compounds were prepared at a concentration of 30 mM. Fifteen nL of compounds/DMSO were transferred to a multi-well plate. Then, 7.5 ul enzyme SRPK1 was added to each well, the plate was spun down at 1000 rpm and centrifuged for 30 sec. Then, 7.5 ul substrate Human SRSF1/SF2 was added to each well, the plate spun down at 1000 rpm and centrifuged for 30 sec. The plate was then incubated at 25° C. for 90 min. After 90 min, 5 μl of ADP-Glo™ Reagent was added to each well and the plate incubated at room temperature for 60 minutes. Then, 10 μl of Kinase Detection Reagent was added to each well and the plate incubate at room temperature for 60 minutes. The luminescence of each well was recorded on Envision. The test compounds were at 30 μM top, 3-fold, in duplicate, and the final concentration of DMSO was 0.1%.

Method 2: The assay buffer was prepared. The reference compound was diluted to 1 mM in the assay buffer. The test compounds were prepared at a concentration of 100 μM, then diluted 3-fold. Five nL of compounds/DMSO were transferred to 384-well plate. The reference compound is 1000 nM top concentration, 3-fold, 10 dose. The test compounds are 100 nM top concentration, 3-fold, 10 dose. 2.5 ul of enzyme (SRPK1 final concentration at 50 μM) was added to each well, then the plate was spun down at 1000 rpm and centrifuged for 30 sec. After, 2.5 ul substrate (Human SRSF1/SF2 final concentration of 0.15 μM, ATP final concentration of 4 μM) was added to the plate, spun down at 1000 rpm and centrifuged for 30 sec. The plate was then placed in an incubator at 25° C. for 3 h. Then, 5 μl of ADP-Glo™ Reagent was added to each well, and the plate incubated at room temperature for 60 minutes. After 60 min, 10 μl of Kinase Detection Reagent was added to each well, and the plate was incubated at room temperature for 30 minutes. The luminescence of each well was recorded on Envision.

The result of the assay for each compound is reported in the below table. All assays were performed using Method 1, except where indicated with an asterisk (*) in the below table, indicating Method 2 was used. I.A. indicates an IC₅₀ greater than 30 μM.

| | Avg IC₅₀ |
|---|---|
| Example | (nM) |
| 001 | 560 |
| 002 | 309 |
| 003 | 971 |
| 004 | 79 |
| 005 | 41.9 |
| 006 | 1420 |
| 007 | 252 |
| 008 | I.A. |
| 009 | I.A. |
| 010 | 8240 |
| 011 | 7490 |
| 012 | 109 |
| 013 | 14700 |
| 014 | I.A. |
| 015 | 3.98* |
| 016 | I.A. |
| 017 | 0.475* |
| 018 | 0.942* |
| 019 | 217 |
| 020 | 0.248* |
| 021 | 13.5 |
| 022 | 1.4* |
| 023 | 55.1 |
| 024 | 346 |
| 025 | 170 |
| 026 | 358 |
| 027 | 36.7 |
| 028 | 105 |
| 029 | 30.9 |
| 030 | 232 |
| 031 | 360 |
| 032 | 114 |
| 033 | 202 |
| 034 | 24.8 |
| 035 | I.A. |
| 036 | I.A. |
| 037 | I.A. |
| 038 | 24500 |
| 039 | I.A. |
| 040 | 193 |
| 041 | I.A. |
| 042 | I.A. |
| 043 | I.A. |
| 044 | 154 |
| 045 | 84.5 |
| 046 | I.A. |
| 047 | 260 |
| 048 | 24.9* |
| 049 | 2340 |
| 050 | 435 |
| 051 | I.A. |
| 052 | 50.2 |
| 053 | 1.98* |
| 054 | 18.5 |
| 055 | 45.1 |
| 056 | 23.5 |
| 057 | 30.5 |
| 058 | 15700 |
| 059 | 78.7 |
| 060 | 51.9 |
| 061 | 8200 |
| 062 | I.A. |
| 063 | 13500 |
| 064 | 249 |
| 065 | 204 |
| 066 | I.A. |
| 067 | |
| 068 | I.A. |
| 069 | 63.7 |
| 070 | 4640 |
| 071 | 6870 |
| 072 | 754 |
| 073 | 59.3 |
| 074 | 641 |
| 075 | 13.1 |
| 076 | 14.2 |
| 077 | I.A. |
| 078 | 27.9 |
| 079 | 102 |
| 080 | 344 |
| 081 | 215 |
| 082 | 55.5 |
| 083 | 39.8 |
| 084 | 60.7 |
| 085 | 2050 |
| 086 | 0.615* |
| 087 | 249 |
| 088 | 71.7 |
| 089 | 878 |
| 090 | 1380 |
| 091 | I.A. |
| 092 | 3.38* |
| 093 | 1260 |
| 094 | I.A. |
| 095 | 0.769* |
| 096 | 18.3 |
| 097 | 3.23* |
| 098 | 871 |
| 099 | 86.3 |
| 100 | 59.7 |
| 101 | I.A. |
| 102 | 340 |
| 103 | 136 |
| 104 | 556 |
| 105 | 1530 |
| 106 | 142 |
| 107 | 746 |
| 108 | 14.8 |
| 109 | 746 |
| 110 | 16.1 |
| 111 | 39.4 |
| 112 | I.A. |
| 113 | I.A. |
| 114 | I.A. |
| 115 | I.A. |
| 116 | I.A. |
| 117 | I.A. |
| 118 | I.A. |
| 119 | I.A. |
| 120 | I.A. |
| 121 | I.A. |
| 122 | I.A. |
| 123 | I.A. |
| 124 | I.A. |
| 125 | I.A. |
| 126 | I.A. |
| 127 | I.A. |
| 128 | I.A. |
| 129 | I.A. |
| 130 | I.A. |
| 131 | I.A. |
| 132 | I.A. |
| 133 | I.A. |
| 134 | I.A. |
| 135 | I.A. |
| 136 | 36 |
| 137 | 210 |

1. A compound having a structure of Formula (I): 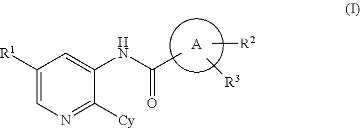
wherein
ring A is a 5-10-membered heterocycle having 1, 2, or 3 ring heteroatoms independently selected from N, O, and S;
Cy is a 4-10-membered heterocycle having 1, 2, or 3 ring heteroatoms independently selected from N, O, and S and optionally substituted with 1 to 4 R^{A};
each R^{A} is independently halo, OH, oxo, C₁₋₆alkyl, C₁₋₆haloalkyl, C₁₋₆hydroxyalkyl, C₀₋₆alkylene-N(R^{N})₂, C₀₋₆alkylene-C(O)N(R^{N})₂, C₀₋₆alkylene-OC(O)C₁₋₆alkyl, C₀₋₆alkylene-C(O) C₁₋₆alkyl, C₀₋₆alkylene-CO₂R^{N}, or C₀₋₆alkylene-4-8-membered heterocycle having 1, 2, 3 or 3 ring heteroatoms independently selected from N, O, and S;
R¹ is H, halo, C₁₋₆alkyl, C₁₋₆haloalkyl, C₁₋₆alkoxy, C₁₋₆haloalkoxy, or C₃₋₆cycloalkyl;
R² is halo, C₁₋₆alkoxy, C₁₋₆haloalkoxy, C₁₋₆alkyl, C₂₋₆alkene, C₂₋₆alkyne, C₁₋₆haloalkyl, CO₂H, or Het;
Het is a 5-8-membered heterocycle having 1, 2, or 3 ring heteroatoms independently selected from N, O, and S, and is optionally substituted with 1 or 2 R^{B};
each R^{B} is independently halo, OH, oxo (═O), C₁₋₆alkyl, C₁₋₆haloalkyl, C₁₋₆alkoxy, C₁₋₆haloalkoxy, C₀₋₆alkylene-N(R^{N})₂, or C₀₋₆alkylene-4-8-membered heterocycle having 1, 2, 3 or 3 ring heteroatoms independently selected from N, O, and S;
R³ is H, halo, or C₁₋₆alkyl; and
each R^{N} is independently H or C₁₋₃alkyl, or a pharmaceutically acceptable salt thereof.

2. The compound or salt of claim 1, wherein ring A is aromatic, and optionally is a 5- or 6-membered ring, or is furan, oxazole, isoxazole, thiophene, thiazole, imidazole, triazole, pyridine, pyrimidine, pyridazine, or pyrazine.

3.-5. (canceled)

6. The compound or salt of claim 1, having a structure of Formula (II) or a structure of Formula (III):

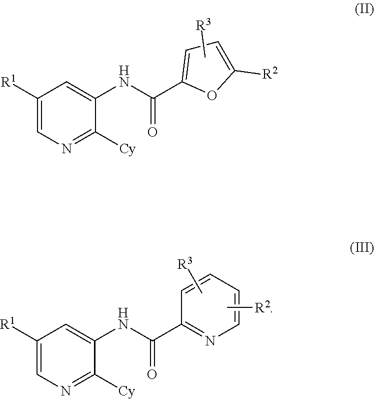

7.-8. (canceled)

9. The compound or salt of claim 1, wherein Cy is azetidine, pyrrolidine, piperidine, piperazine, azepane, morpholine, thiomorpholine, pyrazole, pyridine, pyrazine, pyrimidine, pyridazine, 2,4,6,7-tetrahydro-pyrazolo[4,3-c]pyridine, 2-oxa-7-azaspiro[3.5]nonane, 7-azaspiro[3.5]nonane, 2-oxa-8-azaspiro[4.5]decane, 2,8-diazaspiro[4.5]decane, or 1,8-diazaspiro[4.5]decane.

10. The compound or salt of claim 1, wherein Cy is unsubstituted; or
Cy is substituted with 1 R^{A} or with 2 R^{A}, and at least one R^{A} is OH, F, CH₃, C(O)N(R^{N})₂, CH₂OH, oxo, CF₃, OC(O)CH₃, CO₂CH₃, CO₂H, CH₂-pyridine, C(O)CH₃, or CH₂N(R^{N})₂.

11.-13. (canceled)

14. The compound or salt of claim 1, wherein Cy is

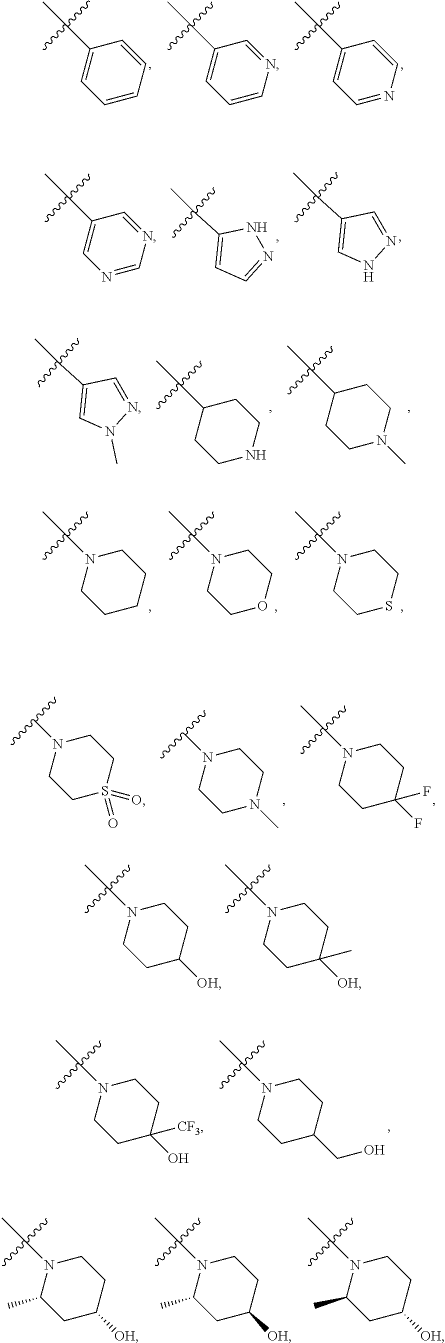

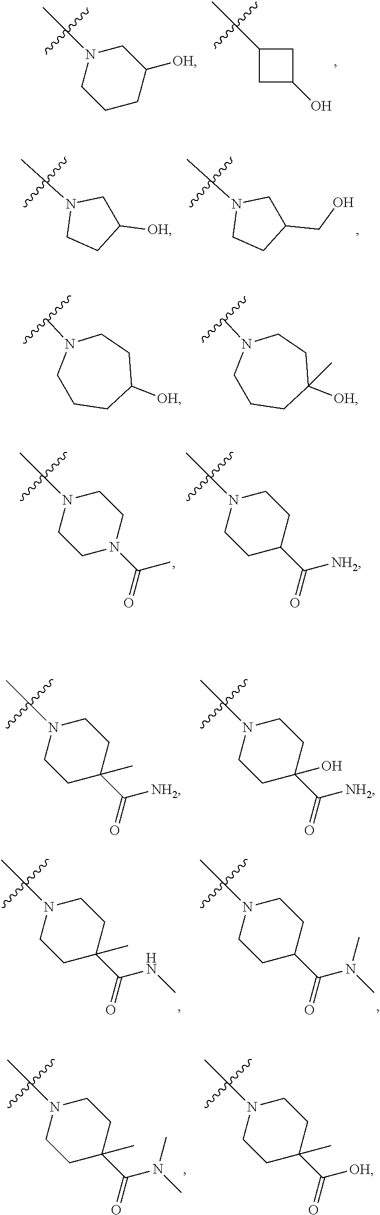

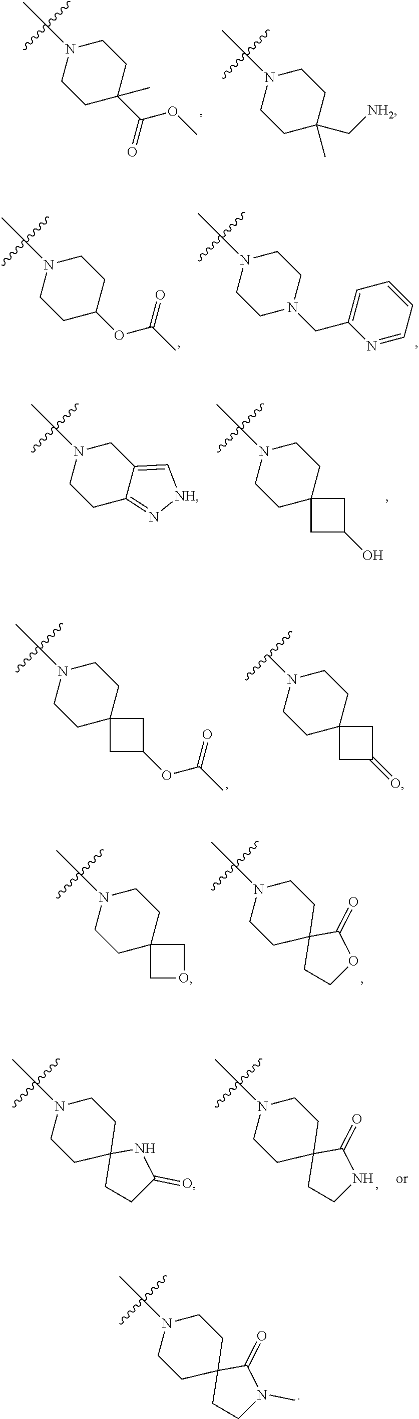

15. The compound or salt of claim 1, wherein R¹ is halo, CF₃, or cyclopropyl.

16.-17. (canceled)

18. The compound or salt of claim 1, wherein R² is halo, C₁₋₃alkoxy, or C₁₋₃haloalkyl.

19. The compound or salt of claim 1, wherein R² is Het, and Het is pyridine, pyrazole, tetrahydropyran, pyrazine, pyrimidine, pyridazine, or piperidine.

20. (canceled)

21. The compound or salt of claim 19, wherein Het is unsubstituted; or
Het is substituted with 1 R^{B} or is substituted with 2 R^{B}, and at least one R^{B} is NH₂, OH, CH₃, or F.

22.-24. (canceled)

25. The compound or salt of claim 1, wherein Het is

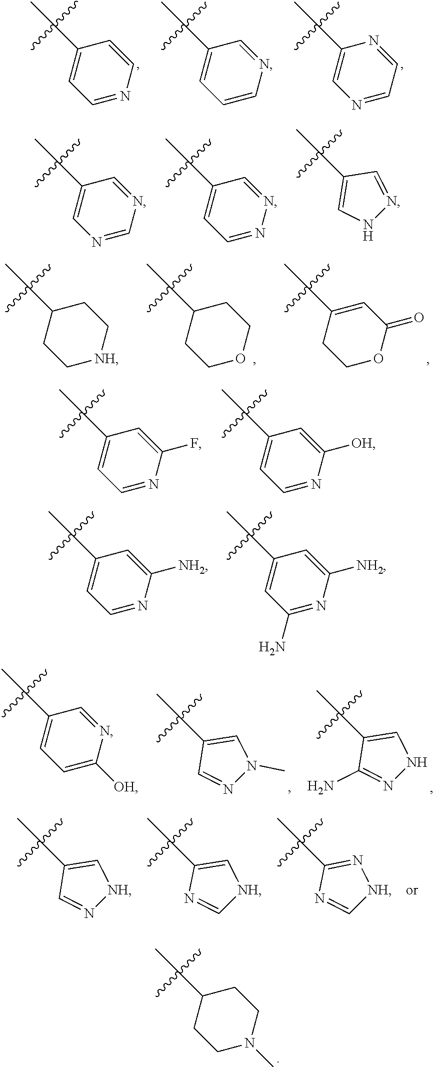

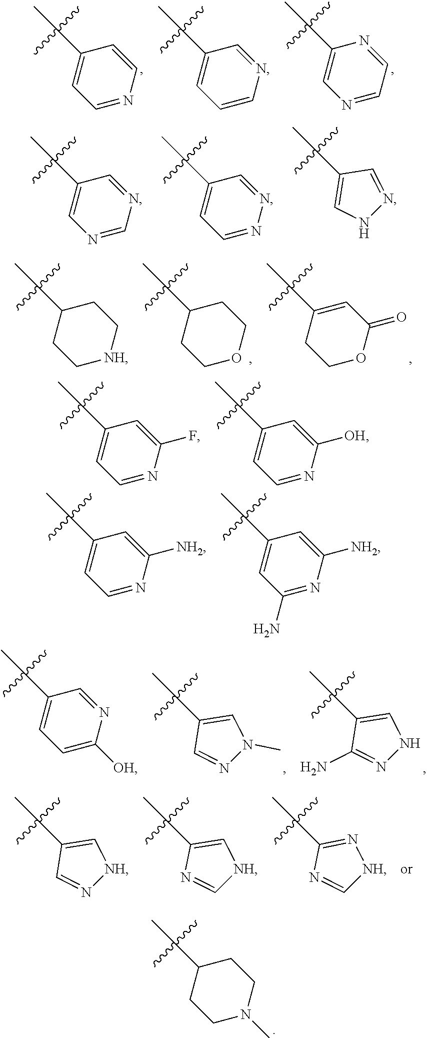

26. The compound or salt of claim 1, wherein R³ is H.

27. A compound as listed in Table A, or a pharmaceutically acceptable salt thereof.

28. A pharmaceutical composition comprising the compound or salt of claim 1 and a pharmaceutically acceptable excipient.

29. A method of inhibiting SRPK1 comprising contacting SRPK1 with an effective amount of the compound or salt of claim 1 to inhibit SRPK1.

30. Use of the compound or salt of claim 1 as a SRPK1 inhibitor.

31. The compound or salt of claim 1 for use as a medicament.

32. A method of treating a subject suffering from a disease or disorder associated with aberrant SRPK1 activity comprising administering to the subject a therapeutically effective amount of the compound or salt of claim 1.

33. The method of claim 32, wherein the disease or disorder is vascular disease, vasoconstriction and disorders characterized by vasoconstriction, cardiovascular disease, malignant or benign neoplasia, (e.g. angiogenesis-dependent cancers, for example tumorous cancers, tumor metastasis, an inflammatory disorder, diabetes, diabetic retinopathy, diabetic neovascularization, diabetic macular edema, trachoma, retrolental hyperplasia, neovascular glaucoma, age-related macular degeneration, wet age-related macular degeneration (wAMD), macular edema, hemangioma, immune rejection of implanted corneal tissue, corneal angiogenesis associated with ocular injury or infection, Osier-Webber Syndrome, myocardial angiogenesis, wound granulation, telangiectasia, hemophiliac joints, angiofibroma, telangiectasia psoriasis scleroderma, pyogenic granuloma, rubeosis, obesity, arthritis (e.g., rheumatoid arthritis, hematopoiesis, vasculogenesis, gingivitis, atherosclerosis, endometriosis, neointimal hyperplasia, psoriasis, hirsutism, proliferative retinopathy, idiopathic pulmonary fibrosis, or diabetic nephropathy.

34. The method of claim 32, wherein the disease or disorder is cancer.

35. (canceled)
